(19) United States
(12) Patent Application Publication
(10) Pub. No.: US 2024/0335429 A1
(43) Pub. Date: Oct. 10, 2024

(54) MOLECULAR GLUE AND SALL4 DEGRADER

(71) Applicants: National University of Singapore, Singapore (SG); Beth lsrael Deaconess Medical Center, Inc., Boston, MA (US)

(72) Inventors: Le Kim Anh VU, Singapore (SG); Bee Hui LIU, Singapore (SG); Daniel Geoffrey TENEN, Singapore (SG)

(21) Appl. No.: 18/293,684

(22) PCT Filed: Aug. 2, 2022
(86) PCT No.: PCT/SG2022/050551

§ 371 Date: Jan. 30, 2024

Related U.S. Application Data
(60) Provisional application No. 63/228,240, filed on Aug. 2, 2021.

(51) Int. Cl. A61K 31/433; A61K 31/17; A61K 31/381; A61K 31/4196; A61K 31/423; A61K 31/428; A61K 31/4545; A61K 31/47; A61P 35/00

(52) U.S. Cl. CPC A61K 31/433; A61K 31/17; A61K 31/381; A61K 31/4196; A61K 31/423; A61K 31/428; A61K 31/4545; A61K 31/47; A61P 35/00

(57) ABSTRACT

Provided is the use of compounds of Formula I, as well as certain specific compounds, or a pharmaceutically acceptable salt, solvate or derivative thereof, in the preparation of a medicament to treat cancer, such as liver cancer (e.g. hepatocellular carcinoma) and lung cancer.

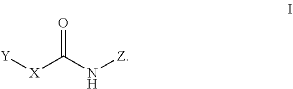

### FIELD OF THE INVENTION

The invention relates to the use of a compound of formula I, and certain specific compounds, and to pharmaceutically acceptable salts, solvates and derivatives thereof, in the preparation of a medicament to treat cancer.

### BACKGROUND

The listing or discussion of an apparently prior-published document in this specification should not necessarily be taken as an acknowledgement that the document is part of the state of the art or is common general knowledge.

Targeted Protein Degradation (TPD) is an emerging technology in drug discovery to achieve the chemical knockdown of pathological protein by hijacking the intracellular proteolysis machinery. This novel drug development strategy is useful for targeting currently intractable protein targets, such as transcription factors. The oncofetal protein SALL4 (Spalt-like transcription factor 4) is a C2H2 zinc finger transcription factor expressed in the embryo and has a fundamental role in regulating stemness genes expression contributing to self-renewal, migration, and anti-apoptosis. In most adult human tissue, SALL4 is silenced and has been found to re-expressed in about one-third of various human cancers. A high expression level of SALL4 is associated with more aggressive disease, poor overall survival, and metastasis in HCC, lung, and endometrial patients, indicating a valuable prognostic potential of SALL4. It has been demonstrated that downregulation of SALL4 expression by interfering RNA in human and murine models of HCC, endometrial cancer, myeloid leukemia, and gastric cancer leads to potent anti-proliferative response and tumor regression. These studies demonstrate the critical role of SALL4 in driving tumorigenesis and cancer cell survival and is thus a promising therapeutic target for cancer treatment. Since SALL4 is silenced in most adult tissues and often reactivated in cancers, there would be minimal tissue toxicity if we aim to degrade the SALL4 protein altogether for adult cancer treatment.

Currently, immunomodulatory drugs (IMiDs) are the only known class of compounds reported to induce CRBN-mediated degradation of SALL4. When tested in SALL4-positive cancer cells, IMiDs had no inhibitory effect on cancer cell growth as expected from the function of this gene. Further studies have revealed an isoform-mediated resistance to IMiDs and the discovery of a more effective degrader of the oncogenic isoform in treating cancer.

### SUMMARY OF THE INVENTION

According to a first aspect of the invention, there is provided:

1. Use of a compound of formula I or a pharmaceutically acceptable salt, solvate or derivative thereof, in the preparation of a medicament to treat cancer, wherein the compound of formula I has the structure

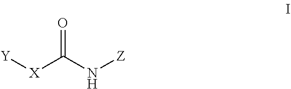

where:
X represents a bond or NH;
Y represents:

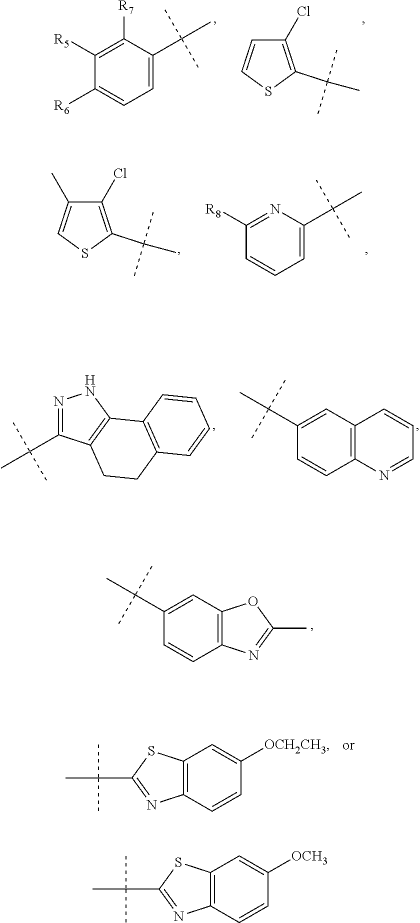

where the dotted lines represent the point of attachment to the rest of the molecule; Z represents:

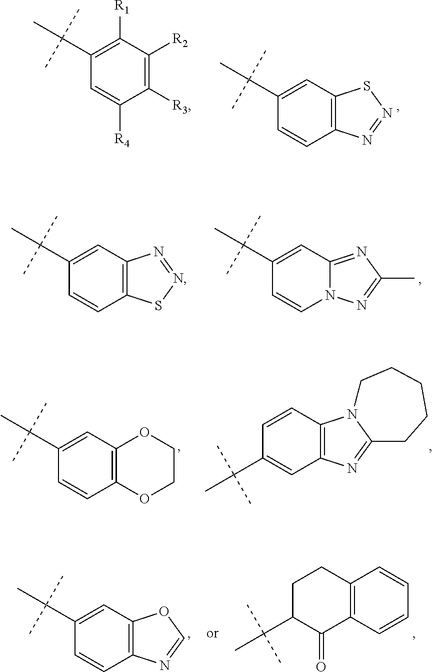

R¹ represents H, Cl, F, OCF₃, OCH₃ or NO₂;
R² represents H, Cl, or OCH₃;
R³ represents H, F, CF₃, OCF₃, CH₃, OCH₃; CH₂CH₃, Cl, —C(═O)OCH₃ or NO₂;
R⁴ represents H, Cl or CH₃;
R⁵ represents H, Cl or F;
R⁶ represents H, CF₃, NO₂, or N (CH₃)₂,

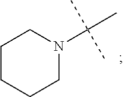

R⁷ represents H or OH;
R⁸ represents H or CH₃; the compound is not

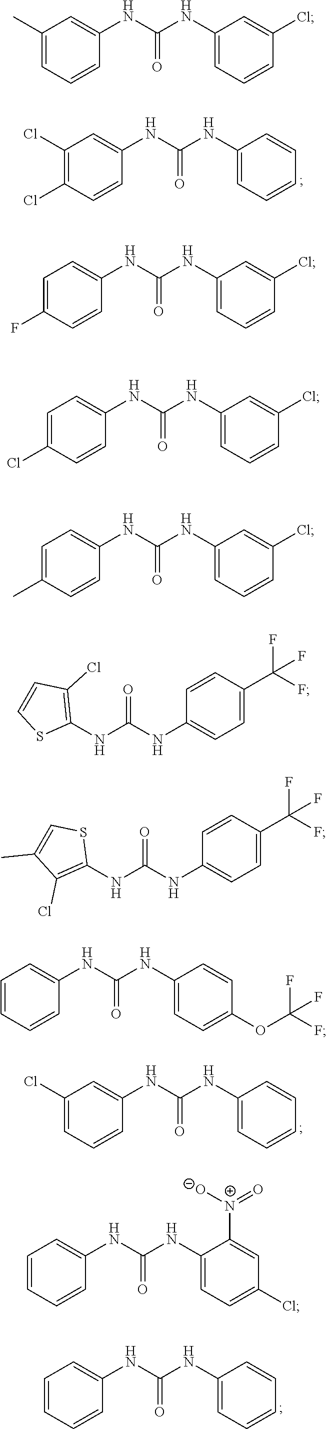

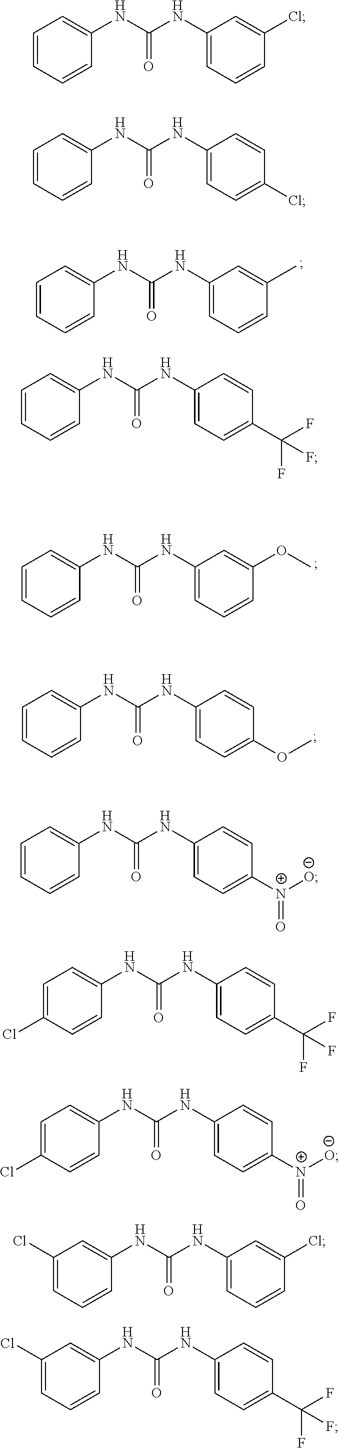

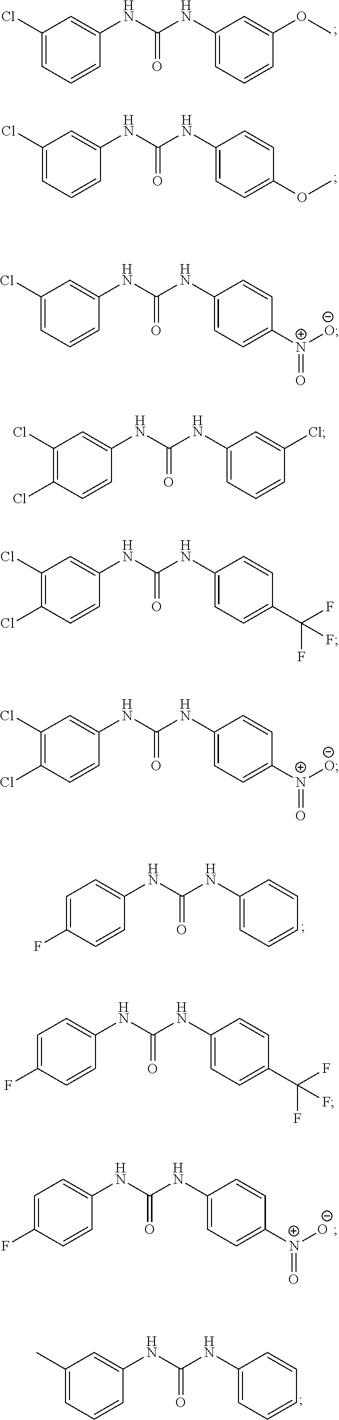

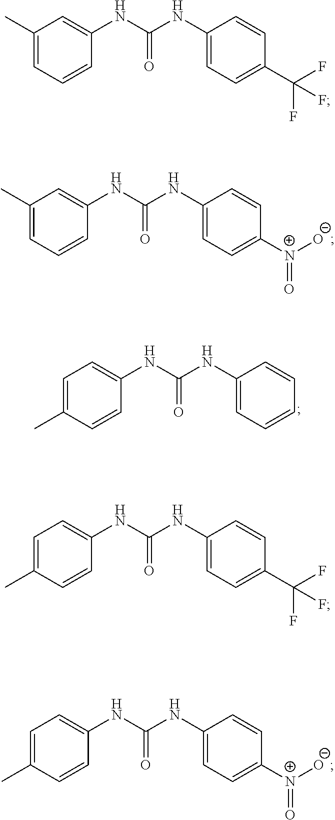 [SMILES: Cc1cccc(NC(=O)Nc2ccc(C(F)(F)F)cc2)c1 ; Cc1cccc(NC(=O)Nc2ccc([N+](=O)[O-])cc2)c1 ; Cc1ccc(NC(=O)Nc2ccccc2)cc1 ; Cc1ccc(NC(=O)Nc2ccc(C(F)(F)F)cc2)cc1 ; Cc1ccc(NC(=O)Nc2ccc([N+](=O)[O-])cc2)cc1 ; ";"]

the compound is not

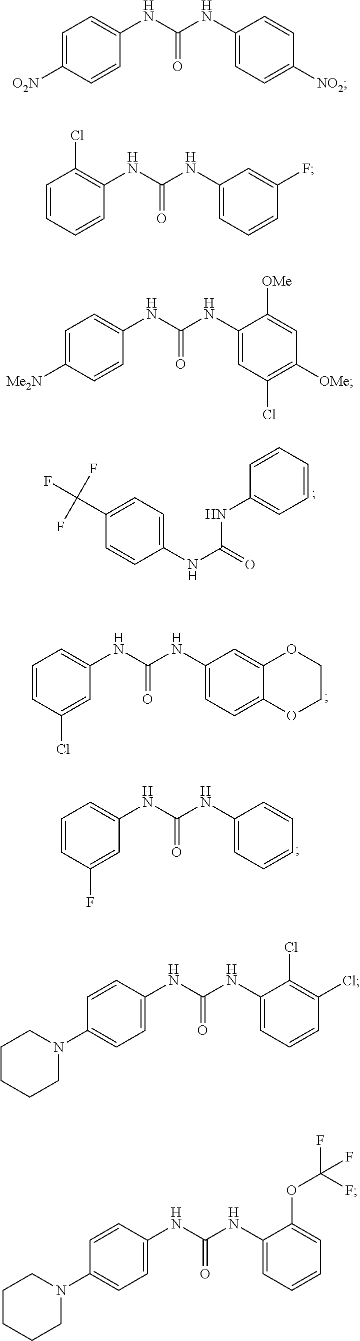

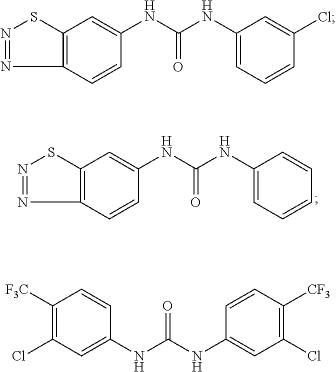 [SMILES: O=C(Nc1cccc(Cl)c1)Nc1ccc2nnsc2c1 ; O=C(Nc1ccccc1)Nc1ccc2nnsc2c1 ; O=C(Nc1ccc(C(F)(F)F)c(Cl)c1)Nc1ccc(C(F)(F)F)c(Cl)c1]

at least one of R¹ to R⁷ is not H;
the compound is not 1-(6-methoxybenzo[d]thiazol-2-yl)-3-phenylurea;
the compound is not 1-(4-chlorophenyl)-3-(6-methoxybenzo[d]thiazol-2-yl)urea;
the compound is not 1-(3-fluorophenyl)-3-(6-methoxybenzo[d]thiazol-2-yl)urea;
the compound is not 1-(3,4-dichlorophenyl)-3-(6-methoxybenzo[d]thiazol-2-yl)urea;
the compound is not 1-(2,3-dichlorophenyl)-3-(6-methoxybenzo[d]thiazol-2-yl)urea;
the compound is not 1-(3-chlorophenyl)-3-(2-hydroxyphenyl)urea;
the compound is not 1-(4-chlorophenyl)-3-(2-hydroxyphenyl)urea;
the compound is not 1-(3-fluorophenyl)-3-(6-methoxy-1,3-benzothiazol-2-yl)urea;
the compound is not 1,3-bis(3-chlorophenyl)urea;
the compound is not 1-(3-chlorophenyl)-3-(4-methylphenyl)urea; and
the compound is not 1-(3-chlorophenyl)-3-(2,4-dichlorophenyl)urea.

2. Use according to Statement 1, wherein Z represents:

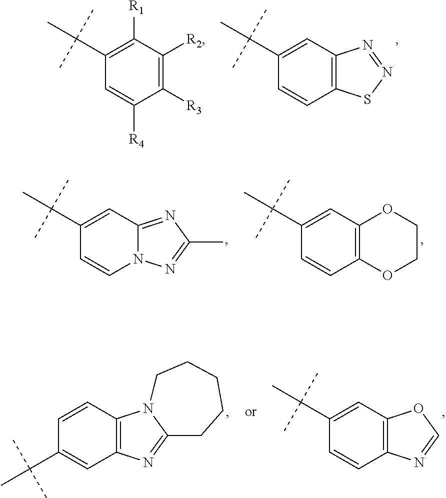

optionally wherein Z represents:

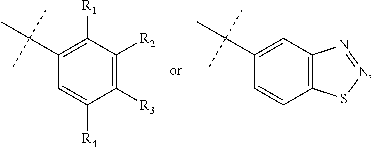

where the dotted lines represent the point of attachment to the rest of the molecule.

3. Use according to Statement 1 or 2 wherein R¹ represents H, Cl, F, OCF₃, or OCH₃, optionally wherein R¹ represents H, Cl, F, or OCH₃.

4. Use according to any one of the preceding statements wherein R² represents H, Cl, or OCH₃.

5. Use according to any one of the preceding statements wherein R³ represents H, F, CF₃, CH₃, OCH₃, Cl, or NO₂, optionally wherein R³ represents H, F, CF₃, CH₃, OCH₃, or Cl.

6. Use according to any one of the preceding statements wherein R⁴ represents H or CH₃, optionally wherein R⁴ represents H.

7. Use according to any one of the preceding statements wherein R⁵ represents H or Cl, optionally wherein R⁵ represents H.

8. Use according to any one of the preceding statements wherein R⁶ represents H, CF₃, or NO₂, optionally wherein R⁶ represents H or NO₂.

9. Use according to any one of the preceding statements wherein R⁷ represents H.

10. Use according to any one of the preceding statements wherein R⁸ represents H.

11. Use according to any one of the preceding statements, wherein X represents NH.

12. Use according to any one of the preceding statements, wherein Y represents:

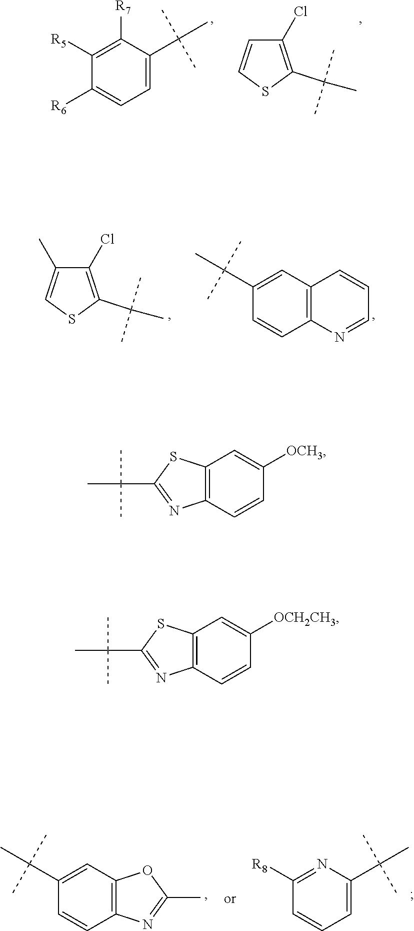

optionally wherein Y represents:

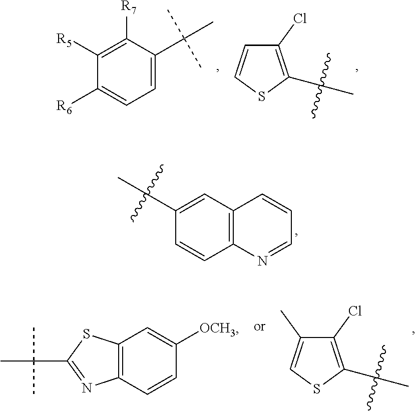

where the dotted lines represent the point of attachment to the rest of the molecule.

13. Use according to any one of Statements 1 to 10, or use according to Statement 12 as dependent on any one of Statements 1 to 10, wherein X represents a bond.

14. Use according to any one of Statements 1 to 10, or use according to Statement 13 as dependent on any one of Statements 1 to 10, wherein Y represents:

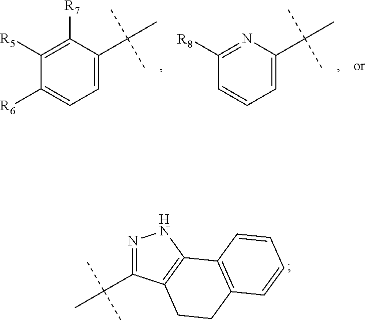

optionally wherein Y represents:

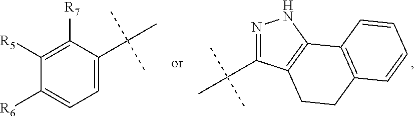

where the dotted lines represent the point of attachment to the rest of the molecule.

The invention also provides the use of a compound having a formula shown in the below Table, or a pharmaceutically acceptable salt, solvate or derivative thereof, in the preparation of a medicament to treat cancer.

| Identifier | Structure |
|---|---|
| XF-84-GG52 | 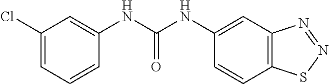 [SMILES: O=C(Nc1cccc(Cl)c1)Nc1ccc2snnc2c1] |
| KE-95-GC86 | 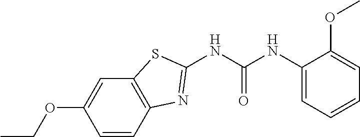 [SMILES: CCOc1ccc2nc(NC(=O)Nc3ccccc3OC)sc2c1] |
| DE-94-ST40 | 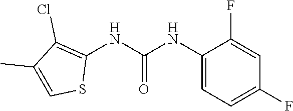 [SMILES: Cc1csc(NC(=O)Nc2ccc(F)cc2F)c1Cl] |
| BC-46-HY47 | 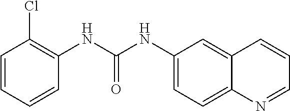 [SMILES: O=C(Nc1ccc2ncccc2c1)Nc1ccccc1Cl] |
| QE-95-GV86 | 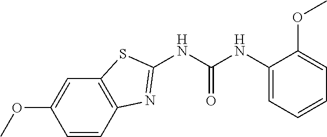 |
| RB-06-1147 | 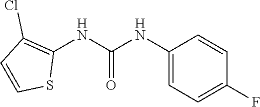 [SMILES: O=C(Nc1ccc(F)cc1)Nc1sccc1Cl] |
| NA-78-BR04 | 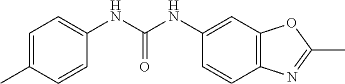 |
| NF-48-LK42 | 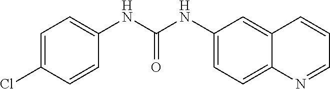 [SMILES: O=C(Nc1ccc(Cl)cc1)Nc1ccc2ncccc2c1] |
| HE-94-SP40 | 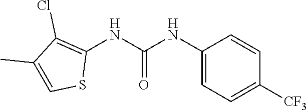 [SMILES: Cc1csc(NC(=O)Nc2ccc(C(F)(F)F)cc2)c1Cl] |
| AE-96-GL86 | 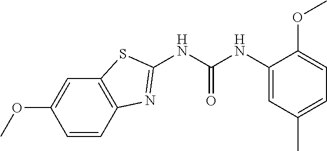 |
| QE-18-WG07 (QE) | 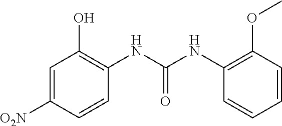 [SMILES: COc1ccccc1NC(=O)Nc1ccc([N+](=O)[O-])cc1O] |
| ZB-04-ZW40 | 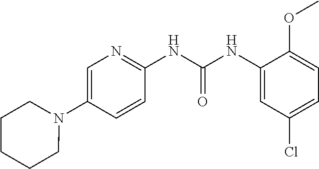 [SMILES: COc1ccc(Cl)cc1NC(=O)Nc1ccc(N2CCCCC2)cn1] |
| TD-57-GF27 | 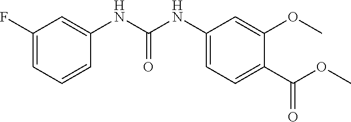 [SMILES: COC(=O)c1ccc(NC(=O)Nc2cccc(F)c2)cc1OC] |
| RD-10-AY51 | 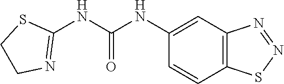 [SMILES: O=C(NC1=NCCS1)Nc1ccc2snnc2c1] |
| QB-77-CJ52 | 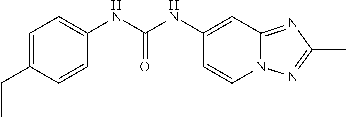 [SMILES: CCc1ccc(NC(=O)Nc2ccn3nc(C)nc3c2)cc1] |
| DC-23-NP48 | 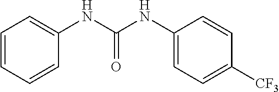 [SMILES: O=C(Nc1ccccc1)Nc1ccc(C(F)(F)F)cc1] |
| PB-97-IJ47 | 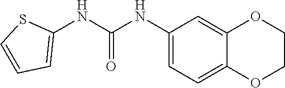 [SMILES: O=C(Nc1ccc2c(c1)OCCO2)Nc1cccs1] |
| VE-35-SA30 | 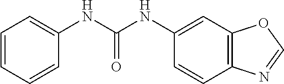 [SMILES: O=C(Nc1ccccc1)Nc1ccc2ncoc2c1] |
| ND-41-RW44 | 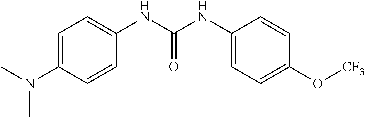 [SMILES: CN(C)c1ccc(NC(=O)Nc2ccc(OC(F)(F)F)cc2)cc1] |
| WF-49-BT50 | 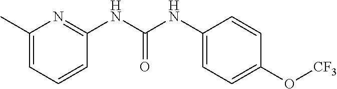 [SMILES: Cc1cccc(NC(=O)Nc2ccc(OC(F)(F)F)cc2)n1] |
| BC-70-JK44 | 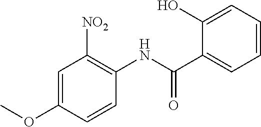 [SMILES: COc1ccc(NC(=O)c2ccccc2O)c([N+](=O)[O-])c1] |
| ZB-20-VG22 | 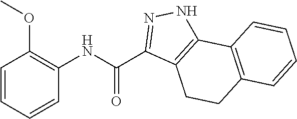 [SMILES: COc1ccccc1NC(=O)c1n[nH]c2c1CCc1ccccc1-2] |
| DE-70-GX95 | 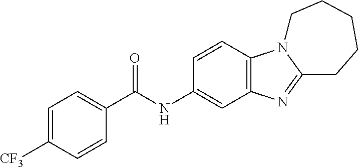 [SMILES: O=C(Nc1ccc2c(c1)nc1n2CCCCC1)c1ccc(C(F)(F)F)cc1] |
| TC-04-QN39 | 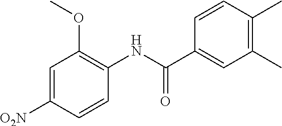 [SMILES: COc1cc([N+](=O)[O-])ccc1NC(=O)c1ccc(C)c(C)c1] |
| BB-39-VG21 | 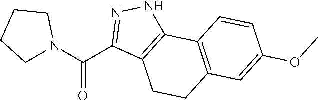 [SMILES: COc1ccc2c(c1)CCc1c(C(=O)N3CCCC3)n[nH]c1-2] |
| PE-30-GL85 | 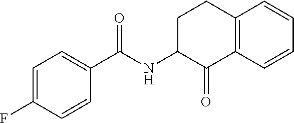 [SMILES: O=C(NC1CCc2ccccc2C1=O)c1ccc(F)cc1] |
| DD-00-TD72 | 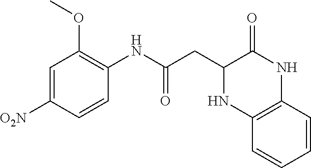 [SMILES: COc1cc([N+](=O)[O-])ccc1NC(=O)CC1Nc2ccccc2NC1=O] |
| WC-23-HW43 | 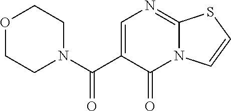 [SMILES: O=C(c1cnc2sccn2c1=O)N1CCOCC1] |
| DA-40-RN26 | 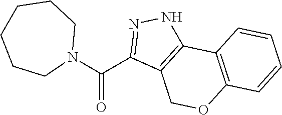 [SMILES: O=C(c1n[nH]c2c1COc1ccccc1-2)N1CCCCCC1] |
| VC-34-CQ41 | 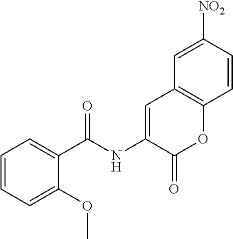 [SMILES: COc1ccccc1C(=O)Nc1cc2cc([N+](=O)[O-])ccc2oc1=O] |
| DE-23-LP28 | 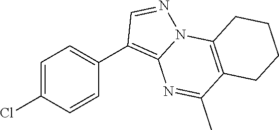 [SMILES: Cc1nc2c(-c3ccc(Cl)cc3)cnn2c2c1CCCC2] |
| HE-23-LL28 | 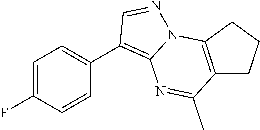 [SMILES: Cc1nc2c(-c3ccc(F)cc3)cnn2c2c1CCC2] |
| VD-47-US03 | 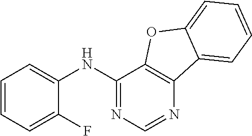 [SMILES: Fc1ccccc1Nc1ncnc2c1oc1ccccc12] |
| RC-42-PC33 | 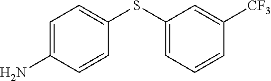 [SMILES: Nc1ccc(Sc2cccc(C(F)(F)F)c2)cc1] |
| RA-02-XM72 | 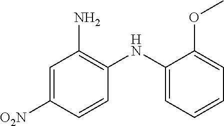 [SMILES: COc1ccccc1Nc1ccc([N+](=O)[O-])cc1N] |
| BD-43-FC37 | 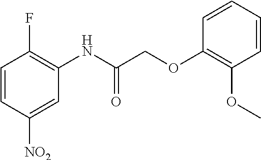 [SMILES: COc1ccccc1OCC(=O)Nc1cc([N+](=O)[O-])ccc1F] |

The invention also provides a compound of formula I and other specific compounds described herein (such as those described in Table A above), or a pharmaceutically acceptable salt, solvate or derivative thereof, as described herein, for use in the treatment of cancer.

The invention also provides a method of treating cancer in a patient in need thereof, said method comprising administering to said patient a therapeutically effective amount of a compound of formula I and other specific compounds described herein (such as those described in Table A above), or a pharmaceutically acceptable salt, solvate or derivative thereof, as described herein.

In some embodiments of the use, compound for use, and method of treatment described above, the cancer may be selected from liver cancer (e.g. hepatocellular carcinoma) and lung cancer.

### BRIEF DESCRIPTION OF THE FIGURES

FIG. 1 shows that IMiDs induce degradation of SALL4A, not SALL4B, in dual-luciferase reporter system. (A) Schematic depiction of the dual luciferase system to evaluate drug-induced SALL4A/B degradation; (B) Immunoblot confirming specific SALL4 or SALL4B expression in H1299 cells overexpressing SALL4A or SALL4B-NanoLuc fusion protein; (C) Normalized ratio of NanoLuc (Nluc)/FireflyLuc (Fluc) signal in H1299 cells stably expressing SALL4A-Nluc or SALL4B-Nluc after 16 h of treatment with IMiDs, Nluc/Fluc ratios were normalized to DMSO-treated cells; (D) Immunoblots showing IMiDs specifically degrade SALL4A-Nluc, not SALL4B-Nluc in H1299 cells after 16 h treatment; and (E) Time-dependent SALL4A degradation induced by 10 μM Pomalidomide treatment in the dual luciferase reporter system.

FIG. 2 shows that IMiDs induce degradation of SALL4A, not SALL4B, without affecting SALL4 mRNA transcript. (A) Human SALL4 gene structure and protein isoforms, with annotated IMiDs binding motifs that are present only in the SALL4A isoform; (B) Effects of IMiDs treatment (12 h, 10 μM) on endogenous SALL4 level in SNU-398 cells were assessed by western blot; (C) Immunoblots showing IMiDs only degrade SALL4A in SNU398 cells after 8 h (n=2); (D) Dose-dependent SALL4A degradation by Thalidomide (i) and Pomalidomide (Pom, ii) in SALL4-positive SNU398 cells; (E) Time-dependent effect of Thalidomide (i) and Pomalidomide (ii) on SALL4 isoforms in SNU398 cells. Only SALL4A is degraded; (F) Quantitative real-time polymerase chain reaction (qRT-PCR) results showing SALL4 mRNA levels in SNU-398 cells were not affected by thalidomide treatment at all time points. Bars represent triplicates mean±SD; and (G) qRT-PCR results showing SALL4 mRNA levels in SNU-398 cells were not affected by Pomalidomide (Pom) treatment at all time points. Bars represent triplicates mean±SD.

FIG. 3 shows that SALL4-mediated cancer cells are insensitive to IMiDs treatment. (A-C) IMiDs did not inhibit (i) SNU-398 and (ii) H661 cell growth (CellTiter-Glo assay). Data represent mean+SD (A-B, n=3; C, n=4).

FIG. 4 shows thalidomide degrades SALL4A, not SALL4B in K562 isogenic lines overexpressing SALL4A or B. (A) Schematic diagram showing design of K562 isogenic lines stably overexpressing wildtype (WT) or zinc-finger-deleted-mutant SALL4A and SALL4B; and (B) SALL4B and ZFC2-deleted SALL4A are resistant to thalidomide-induced degradation after 6 h treatment in K562 isogenic cell lines described in B.

FIG. 5 shows that IMiDs induce proteasomal degradation of SALL4A. (A) Thalidomide depletes endogenous SALL4A, not SALL4B, via proteasomal degradation in HCC (SNU-398) and lung (H661) cancer cells after 6 h treatment, proteasome inhibitor MG132 blocked the SALL4A degradation by IMiDs; and (B) Inhibition of neddylation by 1 uM MLN4924 reverses Thalidomide and Pomalidomide-induced SALL4A degradation in HCC cells.

FIG. 6 shows that SALL4B isoform supports hepatocellular carcinoma (HCC) cell survival and tumorigenesis. (A) Immunoblots showing total SALL4 and SALL4B-specific knockdown in SALL4-high SNU-398 HCC cells by shRNA; (B) Representative images (i-ii) and quantification results (iii-iv) of clonogenic assays in SALL4-high SNU398 compared to SALL4-low SNU-387 cells transduced with scrambled (Scr) or SALL4 shRNA virus. Bar values represent triplicates mean±SD. Student's t-test, *P<0.05, **P<0.01; (C) Viable SNU-398 cell counts post-transduction with scrambled or SALL4 shRNA virus. Data represent triplicates mean±SD; (D-E) Representative images (D) and quantification results (E) of anchorage-independent soft agar colony formation assays in SNU-398 HCC cells transduced with scrambled (Scr) or SALL4 shRNA virus. Bar values represent mean±SD (n=5). Student's t-test, ****P<0.0001; (F-G) Representative image (F) and percentage (G) of Annexin V+ cells analyzed by flow cytometry from SNU398 HCC cells transduced with scrambled or SALL4 shRNA virus on day 5 post-infection. Bars represent triplicates mean±SD. Student's t-test, **P<0.01, ****P<0.0001.

FIG. 7 shows that SALL4-high, not SALL4-low, HCC and non-small-cell lung carcinoma (NSCLC) cell lines are dependent on SALL4B for cell growth. (A) Representative images of clonogenic assays for a panel of HCC Cell Lines transduced with scrambled or SALL4 shRNA virus; (B) Representative images of clonogenic assays for a panel of NSCLC cell Lines transduced with scrambled or SALL4 shRNA virus; (C) Immunoblots showing total SALL4 KD and SALL4B-specific shRNA KD in HCC and NSCLC cells; and (D) Immunoblots showing HCC and NSCLC cell lines with differential SALL4 expression.

FIG. 8 shows that SALL4B transgenic mice demonstrated increased risk of liver tumor development. (A,C) Gross morphology and (B,D) histology following hematoxylin and eosin (H&E) staining of livers of wild type (A,B) and SALL4 transgenic (C,D) mice. Arrow indicates liver tumor. The dashed line in (D) indicates the boundary of normal and tumor (right) region. (B,D) 200×, bar=20 μm; (E) Increased average Liver/Body weight distribution in N-nitrosodiethylamine (DEN)-induced mice in SALL4B over-expressing mice in comparison to wild type mice; and (F) Young SALL4B transgenic mice were vulnerable to chemical carcinogenesis (DEN/phenobarbital (PB)). H&E of (i-iii) wildtype and (iv-vi) SALL4B transgenic livers after 23-week DEN/PB exposure. (iv, v) Arrows show cellular alteration foci. (iii) Magnified regions show interphase cells. (vi) Magnified regions and arrows show increased mitosis. Ki-67 immunohistochemistry staining shows increased proliferation in (viii) SALL4B transgenic livers compared to (vii) wild type livers after 23-weeks DEN/PB exposure. (i, iv) 40×, bar=100 μm. (ii, v) 100×, bar=40 μm. (iii, vi) 400×, bar=1 μm.

FIG. 9 depicts the design of two-stage chemical carcinogenesis experiment. N-nitrosodiethylamine (DEN) and phenobarbital (PB) were used to induce pre-neoplastic hepatic lesions in the wildtype and SALL4B transgenic mice (Tg). Livers were harvested at 10- and 23-weeks (wks) after addition of PB into the drinking water. IP: intraperitoneal, wo: week-old.

FIG. 10 depicts the high-throughput screening (HTS) and hit validation flowchart.

FIG. 11 depicts the summary of thermal shift HTS screen of 50,000 compounds. (A) 1500 thermal shift assay (TSA) hits obtained after primary screen at a single dose of 100 μM; and (B) Confirmed hits consisting of ˜146 positive shifters and ˜737 negative shifters, screened in duplicates, at two concentrations, 50 μM and 100 μM.

FIG. 12 depicts the graphical representation that compares percentage of normalized Nluc/Fluc signal readout of compounds in main screen (x-axis) versus in counter-screen (y-axis). A value of 0 means there is no change in Nluc/Fluc signal read as compared to the DMSO control, whereas a value of −50 indicates a 50% reduction in Nluc/Fluc signal as compared to the DMSO control. The 18 hits were selected in the light orange box, where they result in 50% or more reduction in Nluc/Fluc signal in the main screen (x-axis) with minimal interference of luciferase signal itself in the counter screen (y-axis cut-off at −10 and above).

FIG. 13 shows that the western blot study confirms that the treatment of 15 μM for 16 h of 5 dual-luciferase assay hits result in both endogenous SALL4A and SALL4B protein downregulation in SNU-398 HCC Cell. Beta-actin was used as the internal loading control. Cleaved-poly (ADP-ribose) polymerase (PARP) is the apoptotic marker. C-myc and phosphatase and tensin homolog (PTEN) were monitored as known downstream targets of SALL4.

FIG. 14 depicts (A) the 72 h cell viability response curves of the 5 validated screening hits in a panel of SALL4B-dependent versus independent HCC (top) and NSCLC (bottom) cell lines. Data represent triplicates mean±SD; and (B) Table summarizing 72 h EC₅₀ values of 5 validated hits in various tumor cell lines from A.

FIG. 15 shows that differential SALL4 isoform degradation by QE-18-WG07 from IMiDs contributes to differential biology. (A) Chemical structure of QE-18-WG07 and IMiDs; (B) DMSO-normalized ratio of NanoLuc/Firefly Luciferase signal in H1299 cells stably expressing SALL4B-Nluc after 9-hour treatment with indicated concentrations of QE-18-WG07 or Pomalidomide. Data represent triplicates mean±SD; (C) Representative immunoblot (i) for SALL4, Cleaved PARP, C-myc (downstream target of SALL4) and Beta-actin (Loading control) after 24 hours of treatment of SNU-398 cells with the indicated concentrations of QE-18-WG07 and Thalidomide. (n=6) Prism plots (ii-iii) comparing percentage of protein band density after QE-18-WG07 and Pomalidomide treatment normalized to DMSO control. All the bands were baseline-corrected by normalizing to loading control beta-actin. Quantification was measured using the Immunoblot (i) by Image J, with the calculated half-maximal degradation DC₅₀ values corresponding to each SALL4 isoform; (D) Immunoblots showing endogenous SALL4B degradation after 18 h QE-18-WG07 treatment in SNU-398, SNU-182 and H661 cells (n=2); and (E) QE-18-WG07 inhibits cell growth of SALL4-high SNU-398 HCC cells, whereas IMiDs have no anti-proliferative effect on HCC cells viability (n=3). The 72-h cellular viability was approximated by adenosine triphosphate-dependent luminescence. Data represent triplicates mean±SD.

FIG. 16 shows that QE-18-WG07 is a SALL4 and CRBN molecular glue, mediating SALL4 degradation and anti-viability effects in cancer via a CRBN and proteasome-dependent mechanism. (A) Dependence of thermal stabilization of SALL4 (1-300aa) on concentration of its binding ligand concentration (QE-18-WG07) after 30 min of incubation in thermal shift assay (TSA). Thermal unfolding of SALL4 (1-300) is monitored using SYPRO Orange. Data were collected using 12 increasing QE-18-WG07 concentrations, each in duplicates. Data represent duplicates mean±SD; (B) Representative immunoblot in duplicates for SALL4, Cleaved PARP, C-myc, RBBP4 and Beta-actin (loading control) after a 24-h co-treatment of 16 μM QE-18-WG07 with DMSO, MG132 (3 μM) and MLN4924 (10 μM) in SNU-398 cells (n=3); (C) Immunoblots showing SALL4B degradation by QE-18-WG07 was rescued by 18 h co-treatment with 1 μM MG132 and 15 μM MLN4924 in SNU-398, SNU-182 and H661 cells; (D) (i) The results of immunoblotting of CRBN thermal stabilization by 50 M of QE-18-WG07 as compared to DMSO control sample; and (ii) Illustration of the thermal aggregation curves following ImageJ quantification of the western blots in (i); (E) Isothermal dose-response fingerprints of CRBN stabilization by QE-18-WG07 at 50.6° C., 53.4° C. and 56.1° C.; and (F) Representative immunoblot for SALL4, CRBN, and actin (loading control) after treatment of SNU-398 cells with scrambled shRNA sh-scr (normal CRBN expression) or sh-CRBN (CRBN level is downregulated) for 18 h at the indicated concentrations (n=3).

FIG. 17 shows that QE-18-WG07 anti-cancer effect is dependent on SALL4B. (A) Effect of QE on 72 h viability of HCC and lung cancer cells. Data represent triplicates mean±SD; (B) Fold increase in apoptosis (Caspase-Glo assay) relative to DMSO-treated controls, 20 h co-treatment of QE-18-WG07 with DMSO, 1 μM MG132, or 15 μM MLN4924 in SNU-398, SNU-182, and H661 cells. Bars represent triplicates mean±SD; (C) Immunoblots showing kinetics of SALL4B degradation and PARP cleavage induced by 5 μM QE-18-WG07 in SNU-398 cells (n=2); (D) 48 h co-treatment with 1 μM MG132 or 15 μM MLN4924 abolished QE inhibition of viability of SNU-398, SNU-182, and H661 cells. Data represent triplicates mean±SD; (E) Immunoblots showing PARP cleavage rescue in CRBN-deficient SNU-398 cells after 24 h QE-18-WG07 treatment (n=3); (F) Fold increase in apoptosis (Caspase-Glo assay) relative to DMSO-treated controls, 24 h treatment in SNU-398 cells with normal or deficient CRBN levels. Bars represent triplicates mean±SD; (G) QE-18-WG07 cytotoxicity against SNU-398 cells after 72 h treatment was rescued upon CRBN-knockdown. Data represent triplicates mean±SD; and (H) Overexpression of SALL4B, not SALL4A, conferred QE-18-WG07 resistance in SNU-398 cells (24 h treatment). Data represent mean±SD (n=6).

FIG. 18 shows the rescuing QE-18-WG07 therapeutic effect with SALL4-isoform specific overexpression. (A) qRT-PCR results showing mRNA levels of total SALL4, SALL4A, and SALL4B isoforms in SNU-398 cells transduced with viral particles containing pFUW_SALL4A or B_mcherry or empty plasmid. Bar values represent triplicates mean±SD; and (B) Immunoblots showing SALL4A or B overexpression (OE) in SNU-398 cells transduced with viral particle containing pFUW_SALL4A or B_mcherry or empty plasmid (n=3).

FIG. 19 depicts the proposed mechanism of action for how QE-18-WG07 targets SALL4B for degradation and induces anti-proliferative effects in SALL4-dependent cancer.

FIG. 20 depicts the pilot study showing in vivo anti-tumor effect and tolerance of QE-18-WG07. (A) Tumor volume (means±SEM) in mice after treatment with vehicle (n=8) or 10 mg/kg QE (n=6); (B) Scatter plot of final tumor volume (mean±SEM) after 10-day treatment; (C) Image of tumor size at the conclusion of 10-day pilot study; (D) Treatment with 10 mg/kg of QE-18-WG07 once daily (q.d.) (n=3) did not induce significant weight loss in mice compared to vehicle (n=4) after 10 days. Each data point represents an individual mouse weight; (E) Bar graph depiction (mean±SEM) of spleen weights at conclusion of the 10-day pilot study. Student's t-test, n.s. P>0.05; and (F) Image of spleen size at the conclusion of 10-day pilot study.

FIG. 21 depicts the in vivo anti-tumor effect of QE-18-WG07 in dose-expansion study. (A) Timeline of the dose-expansion study; (B) Tumor volume (means±SEM) in mice after treatment with vehicle (n=11) or QE-18-WG07 at 5, 15 and 45 mg/kg (n=11 per dose group) for 15 days; and (C) Scatter plot of final tumor volume (mean±SEM) after 15-day treatment. Student's t-test, **P<0.01; ****P<0.0001.

FIG. 22 depicts the in vivo anti-tumor effect and tolerance of QE-18-WG07 in dose-expansion study. (A) Treatment with (i) 5; (ii) 15; and (iii) 45 mg/kg of QE-18-WG07 q.d. did not induce significant weight loss in mice after 15 days (n=6 for each treatment group). Each data point represents the weight of an individual mouse; (B) Bar graph depiction (mean±SEM) of spleen weights after 15 days of treatment (n=6 for each treatment group); (C) Bar graph depiction (mean±SEM) of liver weights after 15 days of treatment (n=6 for each treatment group). Student's t-test, ns P>0.05; (D) Image of tumor size after 15 days (n=11 for each treatment group); (E) Image of mice spleen size after 15 days of treatment; and (F) Image of mice liver size after 15 days of treatment.

FIG. 23 depicts the graphical abstract of overcoming isoform-mediated cancer drug resistance.

### DETAILED DESCRIPTION OF THE INVENTION

The word “comprising” refers herein may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

As used herein, the singular forms “a,” “an,” and “the” include plural referents unless the context clearly dictates otherwise. Thus, for example, reference to “a composition” includes mixtures of two or more such compositions, reference to “an oxygen carrier” includes mixtures of two or more such oxygen carriers, reference to “the catalyst” includes mixtures of two or more such catalysts, and the like.

References herein (in any aspect or embodiment of the invention) to compounds of formula I includes references to such compounds per se, to tautomers of such compounds, as well as to pharmaceutically acceptable salts or solvates, or pharmaceutically functional derivatives of such compounds. General references to compounds of formula I herein, unless clearly excluded by context, are also to be understood as covering any specific compounds disclosed herein, whether they fall within the literal scope of formula I or not. For example, general references to compounds of formula I being useful to treat a disease, or to possible salts of compounds of formula I, are to be understood as also covering any specific compound disclosed herein (such as those disclosed in Table A), while a reference to compounds of formula I followed by the structure of formula I and definitions of its substituents is to be understood as covering only compounds falling within that formula.

For the avoidance of doubt, it is herein explicitly noted that any reference to a “compound” in a disclaimer or other form of clause defining negative subject-matter is to be interpreted as covering the compounds (whether identified by structure or name) explicitly disclaimed, and tautomers thereof, as well as pharmaceutically acceptable salts and solvates thereof.

The invention provides the use of a compound of formula I or a pharmaceutically acceptable salt, solvate or derivative thereof, in the preparation of a medicament to treat cancer, wherein the compound of formula I has the structure

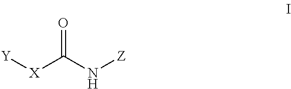

where:
X represents a bond or NH;
Y represents:

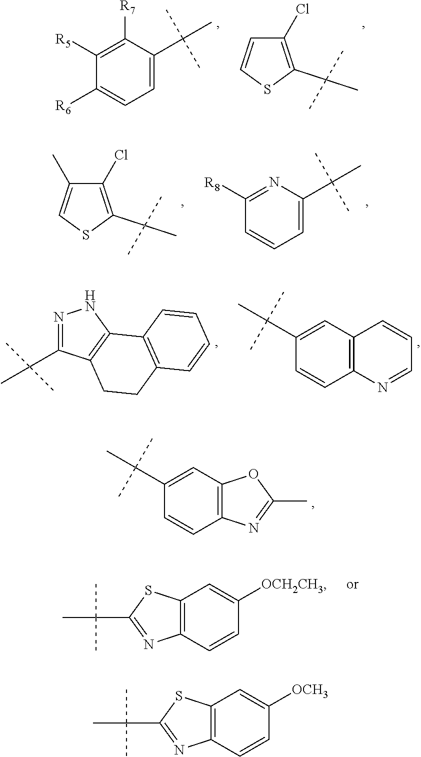

where the dotted lines represent the point of attachment to the rest of the molecule;
Z represents:

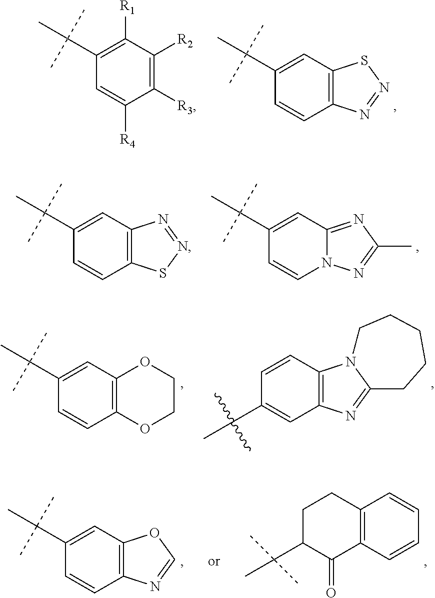

R¹ represents H, Cl, F, OCF₃, OCH₃ or NO₂;
R² represents H, Cl, or OCH₃;
R³ represents H, F, CF₃, OCF₃, CH₃, OCH₃; CH₂CH₃, Cl, —C(═O)OCH₃ or NO₂;
R⁴ represents H, Cl or CH₃;
R⁵ represents H, Cl or F;
R⁶ represents H, CF₃, NO₂, or N(CH₃)₂,

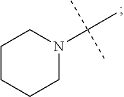

R⁷ represents H or OH;
R⁸ represents H or CH₃;
provided that
the compound is not

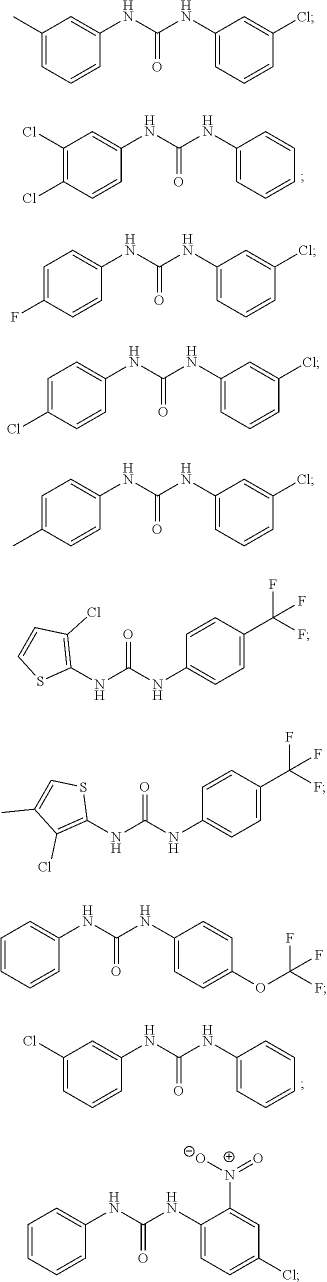

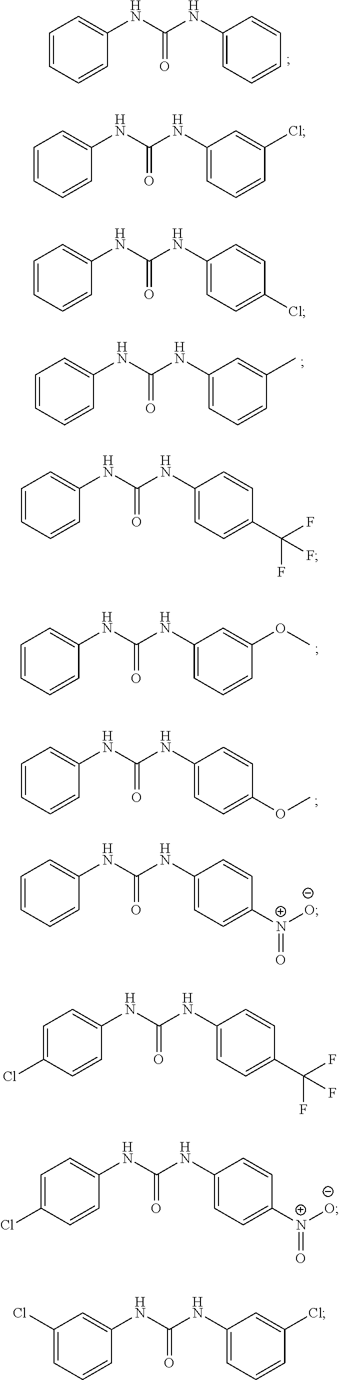

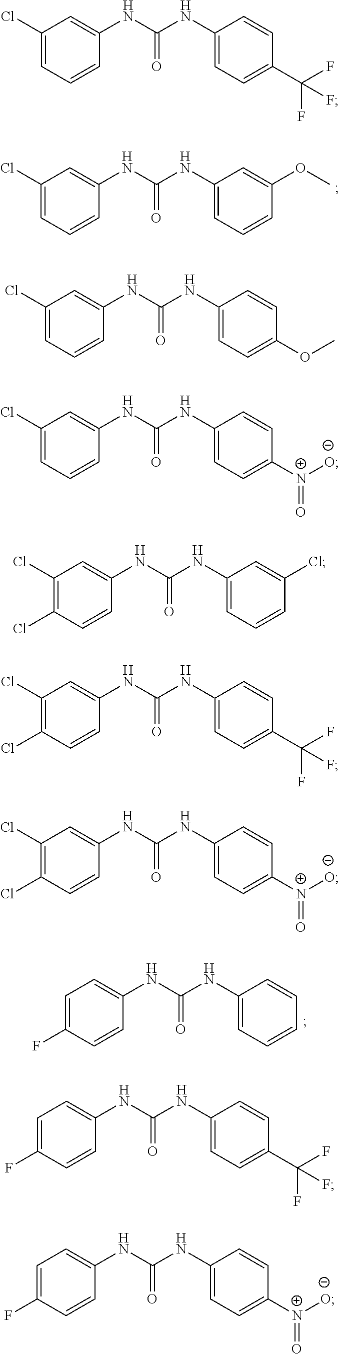

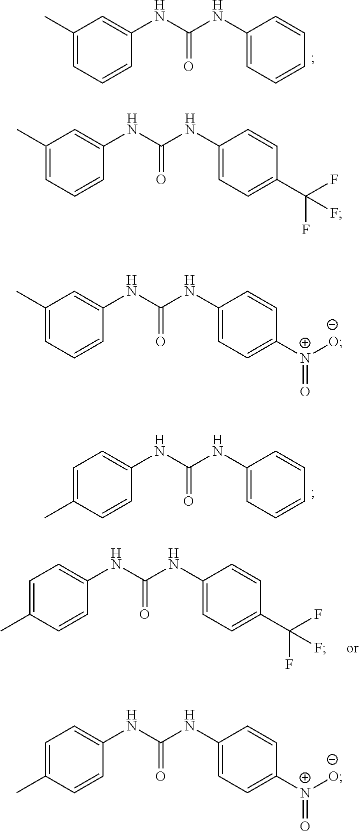

the compound is not

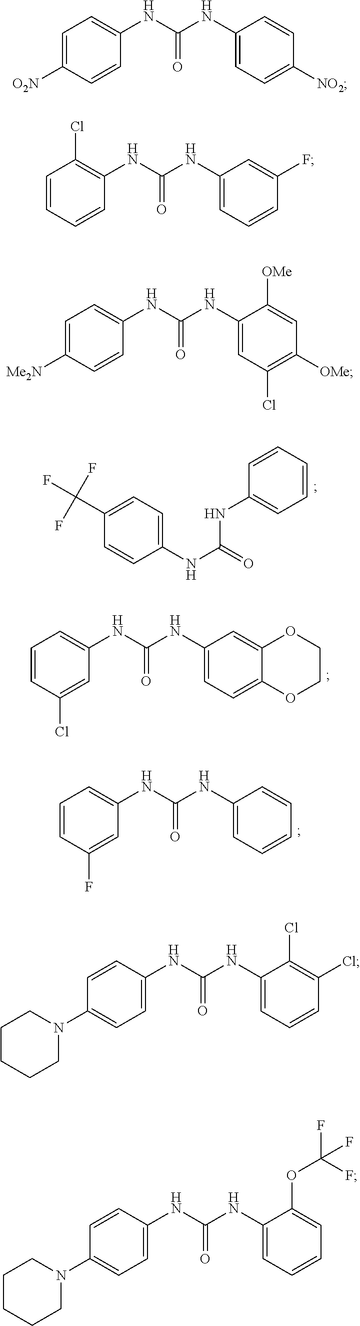

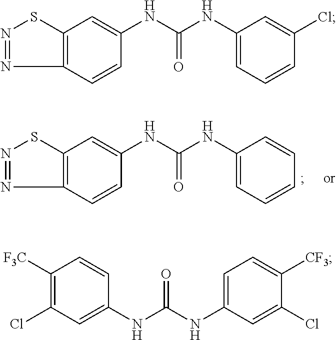 [SMILES: O=C(Nc1cccc(Cl)c1)Nc1ccc2nnsc2c1 ; O=C(Nc1ccccc1)Nc1ccc2nnsc2c1 ; O=C(Nc1ccc(C(F)(F)F)c(Cl)c1)Nc1ccc(C(F)(F)F)c(Cl)c1]

at least one of R¹ to R⁷ is not H;
the compound is not 1-(6-methoxybenzo[d]thiazol-2-yl)-3-phenylurea;
the compound is not 1-(4-chlorophenyl)-3-(6-methoxybenzo[d]thiazol-2-yl)urea;
the compound is not 1-(3-fluorophenyl)-3-(6-methoxybenzo[d]thiazol-2-yl)urea;
the compound is not 1-(3,4-dichlorophenyl)-3-(6-methoxybenzo[d]thiazol-2-yl)urea;
the compound is not 1-(2,3-dichlorophenyl)-3-(6-methoxybenzo[d]thiazol-2-yl)urea;
the compound is not 1-(3-chlorophenyl)-3-(2-hydroxyphenyl)urea;
the compound is not 1-(4-chlorophenyl)-3-(2-hydroxyphenyl)urea;
the compound is not 1-(3-fluorophenyl)-3-(6-methoxy-1,3-benzothiazol-2-yl)urea;
the compound is not 1,3-bis(3-chlorophenyl)urea;
the compound is not 1-(3-chlorophenyl)-3-(4-methylphenyl)urea; and
the compound is not 1-(3-chlorophenyl)-3-(2,4-dichlorophenyl)urea.

In some embodiments of the invention, Z may represent a moiety having one of the below formulae:

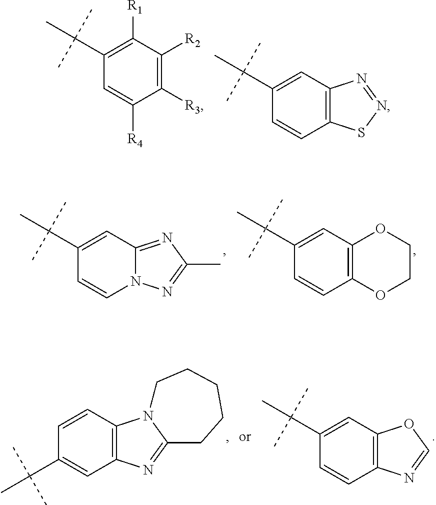

Dotted/dashed lines may be used herein to denote the point of attachment of a moiety to the rest of the molecule. Thus, in the above formulae the dotted lines represent the point of attachment to the rest of the molecule.

In further embodiments of the invention, Z may represent a moiety having one of the below formulae:

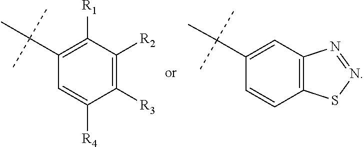

In some embodiments of the invention, R¹ may represent H, Cl, F, OCF₃, or OCH₃. In further embodiments of the invention, R¹ may represent H, Cl, F, or OCH₃.

In some embodiments of the invention, R² may represent H, Cl, or OCH₃.

In some embodiments of the invention, R³ may represent H, F, CF₃, CH₃, OCH₃, Cl, or NO₂. In further embodiments of the invention, R³ may represent H, F, CF₃, CH₃, OCH₃, or Cl.

In some embodiments of the invention, R⁴ may represent H or CH₃. In further embodiments of the invention, R⁴ may represent H.

In some embodiments of the invention, R⁵ represents H or Cl. In further embodiments of the invention, R⁵ may represent H.

In some embodiments of the invention, R⁶ may represent H, CF₃, or NO₂. In further embodiments of the invention, R⁶ may represent H or NO₂.

In some embodiments of the invention, R⁷ may represent H.

In some embodiments of the invention, R⁸ may represent H.

In some embodiments of the invention, X may represent NH.

In some embodiments of the invention (for example those in which X may represent NH), Y may represent a moiety having one of the below formulae:

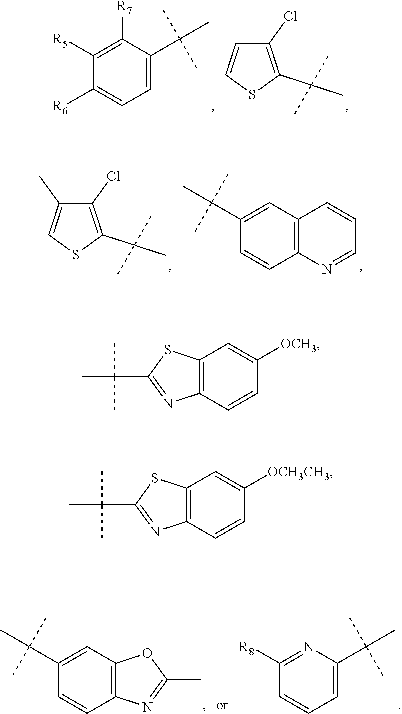

In further embodiments of the invention (for example those in which X may represent NH), Y may represent a moiety having one of the below formulae:

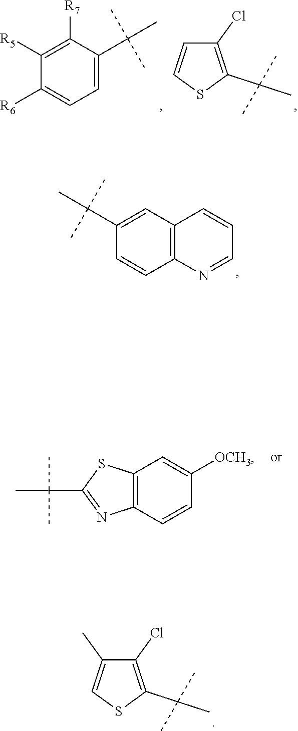

In some embodiments of the invention, X may represent a bond.

In some embodiments of the invention (for example those in which X may represent a bond), Y may represent a moiety having one of the below formulae:

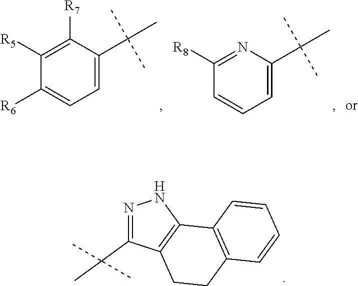

In further embodiments of the invention (for example those in which X may represent a bond), Y may represent a moiety having one of the below formulae:

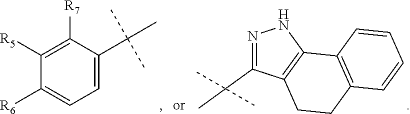

The invention also provides the use of a compound disclosed herein, or a pharmaceutically acceptable salt, solvate or derivative thereof, in the preparation of a medicament to treat cancer.

Thus, the invention provides the use of a compound having a formula shown in the below Table A, or a pharmaceutically acceptable salt, solvate or derivative thereof, in the preparation of a medicament to treat cancer:

TABLE A

| Identifier | Structure |
|---|---|
| XF-84-GG52 | 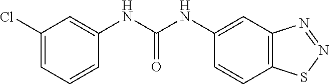 [SMILES: O=C(Nc1cccc(Cl)c1)Nc1ccc2snnc2c1] |
| KE-95-GC86 | 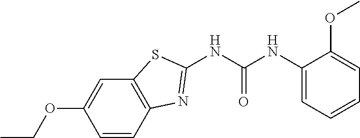 [SMILES: CCOc1ccc2nc(NC(=O)Nc3ccccc3OC)sc2c1] |
| DE-94-ST40 | 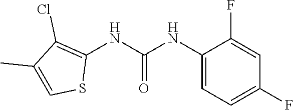 [SMILES: Cc1csc(NC(=O)Nc2ccc(F)cc2F)c1Cl] |
| BC-46-HY47 | 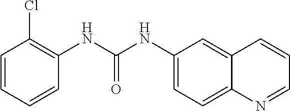 [SMILES: O=C(Nc1ccc2ncccc2c1)Nc1ccccc1Cl] |
| QE-95-GV86 | 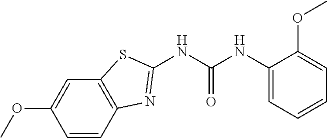 |
| RB-06-II47 | 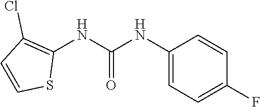 [SMILES: O=C(Nc1ccc(F)cc1)Nc1sccc1Cl] |
| NA-78-BR04 | 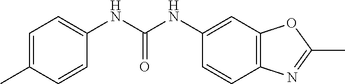 |
| NF-48-LK42 | 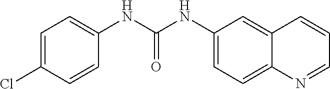 [SMILES: O=C(Nc1ccc(Cl)cc1)Nc1ccc2ncccc2c1] |
| HE-94-SP40 | 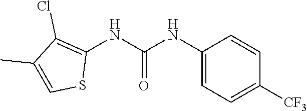 [SMILES: Cc1csc(NC(=O)Nc2ccc(C(F)(F)F)cc2)c1Cl] |
| AE-96-GL86 | 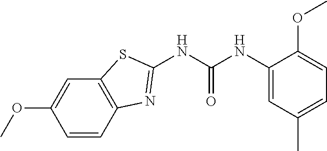 |
| QE-18-WG07 (QE) | 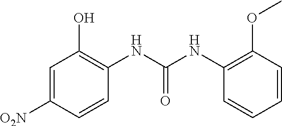 [SMILES: COc1ccccc1NC(=O)Nc1ccc([N+](=O)[O-])cc1O] |
| ZB-04-ZW40 | 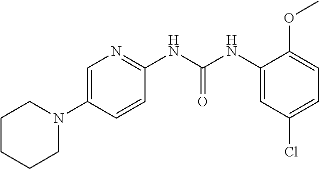 [SMILES: COc1ccc(Cl)cc1NC(=O)Nc1ccc(N2CCCCC2)cn1] |
| TD-57-GF27 | 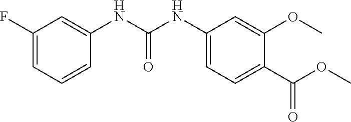 [SMILES: COC(=O)c1ccc(NC(=O)Nc2cccc(F)c2)cc1OC] |
| RD-10-AY51 | 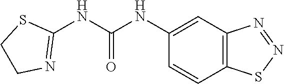 [SMILES: O=C(NC1=NCCS1)Nc1ccc2snnc2c1] |
| QB-77-CJ52 | 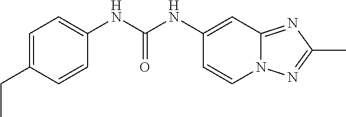 [SMILES: CCc1ccc(NC(=O)Nc2ccn3nc(C)nc3c2)cc1] |
| DC-23-NP48 | 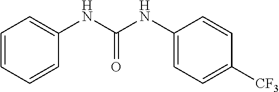 [SMILES: O=C(Nc1ccccc1)Nc1ccc(C(F)(F)F)cc1] |
| PB-97-IJ47 | 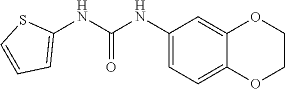 [SMILES: O=C(Nc1ccc2c(c1)OCCO2)Nc1cccs1] |
| VE-35-SA30 | 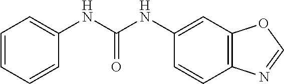 [SMILES: O=C(Nc1ccccc1)Nc1ccc2ncoc2c1] |
| ND-41-RW44 | 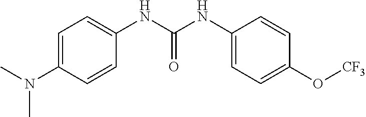 [SMILES: CN(C)c1ccc(NC(=O)Nc2ccc(OC(F)(F)F)cc2)cc1] |
| WF-49-BT50 | 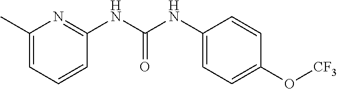 [SMILES: Cc1cccc(NC(=O)Nc2ccc(OC(F)(F)F)cc2)n1] |
| BC-70-JK44 | 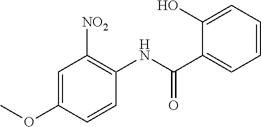 [SMILES: COc1ccc(NC(=O)c2ccccc2O)c([N+](=O)[O-])c1] |
| ZB-20-VG22 | 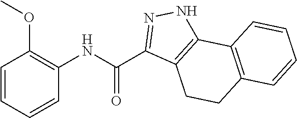 [SMILES: COc1ccccc1NC(=O)c1n[nH]c2c1CCc1ccccc1-2] |
| DE-70-GX95 | 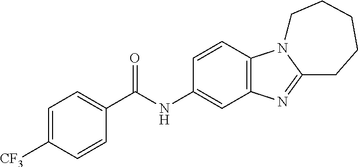 [SMILES: O=C(Nc1ccc2c(c1)nc1n2CCCCC1)c1ccc(C(F)(F)F)cc1] |
| TC-04-QN39 | 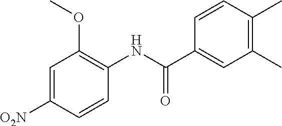 [SMILES: COc1cc([N+](=O)[O-])ccc1NC(=O)c1ccc(C)c(C)c1] |
| BB-39-VG21 | 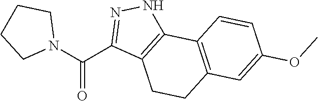 [SMILES: COc1ccc2c(c1)CCc1c(C(=O)N3CCCC3)n[nH]c1-2] |
| PE-30-GL85 | 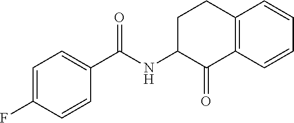 [SMILES: O=C(NC1CCc2ccccc2C1=O)c1ccc(F)cc1] |
| DD-00-TD72 | 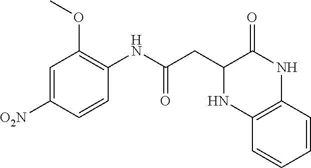 [SMILES: COc1cc([N+](=O)[O-])ccc1NC(=O)CC1Nc2ccccc2NC1=O] |
| WC-23-HW43 | 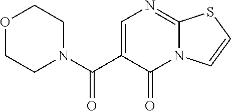 [SMILES: O=C(c1cnc2sccn2c1=O)N1CCOCC1] |
| DA-40-RN26 | 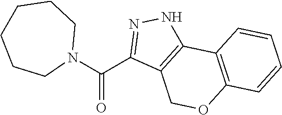 [SMILES: O=C(c1n[nH]c2c1COc1ccccc1-2)N1CCCCCC1] |
| VC-34-CQ41 | 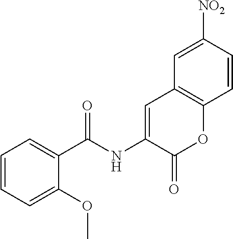 [SMILES: COc1ccccc1C(=O)Nc1cc2cc([N+](=O)[O-])ccc2oc1=O] |
| DE-23-LP28 | 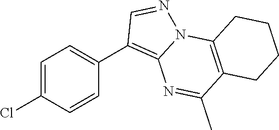 [SMILES: Cc1nc2c(-c3ccc(Cl)cc3)cnn2c2c1CCCC2] |
| HE-23-LL28 | 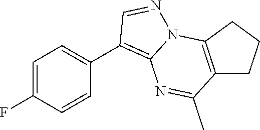 [SMILES: Cc1nc2c(-c3ccc(F)cc3)cnn2c2c1CCC2] |
| VD-47-US03 | 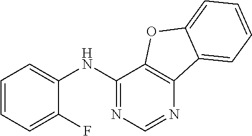 [SMILES: Fc1ccccc1Nc1ncnc2c1oc1ccccc12] |
| RC-42-PC33 | 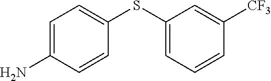 [SMILES: Nc1ccc(Sc2cccc(C(F)(F)F)c2)cc1] |
| RA-02-XM72 | 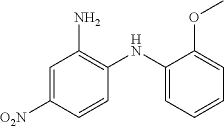 [SMILES: COc1ccccc1Nc1ccc([N+](=O)[O-])cc1N] |
| BD-43-FC37 | 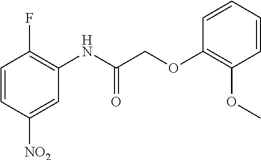 [SMILES: COc1ccccc1OCC(=O)Nc1cc([N+](=O)[O-])ccc1F] |

Pharmaceutically acceptable salts that may be mentioned include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of formula I in the form of a salt with another counter-ion, for example using a suitable ion exchange resin.

Examples of pharmaceutically acceptable salts include acid addition salts derived from mineral acids and organic acids, and salts derived from metals such as sodium, magnesium, or preferably, potassium and calcium.

Examples of acid addition salts include acid addition salts formed with acetic, 2,2-dichloroacetic, adipic, alginic, aryl sulphonic acids (e.g. benzenesulphonic, naphthalene-2-sulphonic, naphthalene-1,5-disulphonic and p-toluenesulphonic), ascorbic (e.g. L-ascorbic), L-aspartic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, gluconic (e.g. D-gluconic), glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±)-DL-lactic), lactobionic, maleic, malic (e.g. (−)-L-malic), malonic, (±)-DL-mandelic, metaphosphoric, methanesulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, tartaric (e.g. (+)-L-tartaric), thiocyanic, undecylenic and valeric acids.

Particular examples of salts are salts derived from mineral acids such as hydrochloric, hydrobromic, phosphoric, metaphosphoric, nitric and sulphuric acids; from organic acids, such as tartaric, acetic, citric, malic, lactic, fumaric, benzoic, glycolic, gluconic, succinic, arylsulphonic acids; and from metals such as sodium, magnesium, or preferably, potassium and calcium.

As mentioned above, also encompassed by formula I are any solvates of the compounds and their salts. Preferred solvates are solvates formed by the incorporation into the solid state structure (e.g. crystal structure) of the compounds of the invention of molecules of a non-toxic pharmaceutically acceptable solvent (referred to below as the solvating solvent). Examples of such solvents include water, alcohols (such as ethanol, isopropanol and butanol) and dimethylsulphoxide. Solvates can be prepared by recrystallising the compounds of the invention with a solvent or mixture of solvents containing the solvating solvent. Whether or not a solvate has been formed in any given instance can be determined by subjecting crystals of the compound to analysis using well known and standard techniques such as thermogravimetric analysis (TGE), differential scanning calorimetry (DSC) and X-ray crystallography.

The solvates can be stoichiometric or non-stoichiometric solvates. Particularly preferred solvates are hydrates, and examples of hydrates include hemihydrates, monohydrates and dihydrates.

For a more detailed discussion of solvates and the methods used to make and characterise them, see Bryn et al., Solid-State Chemistry of Drugs, Second Edition, published by SSCI, Inc of West Lafayette, IN, USA, 1999, ISBN 0-967-06710-3.

“Pharmaceutically functional derivatives” of compounds of formula I as defined herein includes ester derivatives and/or derivatives that have, or provide for, the same biological function and/or activity as any relevant compound of the invention. Thus, for the purposes of this invention, the term also includes prodrugs of compounds of formula I.

The term “prodrug” of a relevant compound of formula I includes any compound that, following oral or parenteral administration, is metabolised in vivo to form that compound in an experimentally-detectable amount, and within a predetermined time (e.g. within a dosing interval of between 6 and 24 hours (i.e. once to four times daily)).

Prodrugs of compounds of formula I may be prepared by modifying functional groups present on the compound in such a way that the modifications are cleaved, in vivo when such prodrug is administered to a mammalian subject. The modifications typically are achieved by synthesizing the parent compound with a prodrug substituent. Prodrugs include compounds of formula I wherein a hydroxyl, amino, sulfhydryl, carboxyl or carbonyl group in a compound of formula I is bonded to any group that may be cleaved in vivo to regenerate the free hydroxyl, amino, sulfhydryl, carboxyl or carbonyl group, respectively.

Examples of prodrugs include, but are not limited to, esters and carbamates of hydroxyl functional groups, esters groups of carboxyl functional groups, N-acyl derivatives and N-Mannich bases. General information on prodrugs may be found e.g. in Bundegaard, H. “Design of Prodrugs” p. I-92, Elsevier, New York-Oxford (1985).

Compounds of formula I, the specific compounds disclosed herein, as well as pharmaceutically acceptable salts, solvates and pharmaceutically functional derivatives of such compounds are, for the sake of brevity, hereinafter referred to together as the “compounds of formula I”.

Compounds of formula I may contain double bonds and may thus exist as E (entgegen) and Z (zusammen) geometric isomers about each individual double bond. All such isomers and mixtures thereof are included within the scope of the invention.

Compounds of formula I may exist as regioisomers and may also exhibit tautomerism. All tautomeric forms and mixtures thereof are included within the scope of the invention.

Compounds of formula I may contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

As explained above, in a first aspect the invention provides the use of a compound of formula I in the manufacture/preparation of a medicament to treat cancer. In further aspects, the invention provides the compound of formula I for use in treating cancer, and a method of treating cancer in a patient in need thereof, said method comprising administering to said patient a therapeutically effective amount of a compound of formula I.

In some embodiments of the invention, the cancer may be selected from one or more of the group consisting of liver cancer (e.g. hepatocellular carcinoma) and lung cancer. In further embodiments of the invention, the cancer may be selected from the group consisting of leukaemia (e.g. acute myeloid leukaemia (AML) or B-cell acute lymphocytic leukaemia (B-ALL)), germ cell tumours, gastric cancer, breast cancer, liver cancer (e.g. hepatocellular carcinoma (HCC)), lung cancer, and glioma.

As explained herein and without being bound by theory, the compounds of the invention are believed to provide anti-cancer effects due to their ability to induce degradation of the SALL4 protein (especially SALL4B) in cancer cells. As explained in more detail herein, SALL4 degradation induced by compounds of formula I is believed to be performed by the E3 ligase cerebron (CRBN). Thus, compounds of formula I are believed to be CRBN modulators.

Thus, according to further aspects of the invention, there is provided:
(i) the use of a compound formula I for the manufacture of a medicament for the treatment of a condition or disorder ameliorated by induction of SALL4 (e.g. SALL4B) degratation; and
(ii) a method of treatment of a disorder or condition ameliorated by inducing the degradation of SALL4 (e.g. SALL4B), which method comprises the administration of an effective amount of a compound of formula I to a patient in need of such treatment.

The term “disorder or condition ameliorated by inducing the degradation of SALL4 (e.g. SALL4B)” will be understood by those skilled in the art to include cancer, such as liver cancer (e.g. hepatocellular carcinoma) and lung cancer.

Thus, further aspects of the invention relate to the following.
(a) A compound of formula I, as hereinbefore defined, for use in the treatment of a condition or disorder selected from cancer, such as liver cancer (e.g. hepatocellular carcinoma) and lung cancer.
(b) Use of a compound of formula I, as hereinbefore defined, for the preparation of a medicament for the treatment of a condition or disorder selected from cancer, such as liver cancer (e.g. hepatocellular carcinoma) and lung cancer.
(c) A method of treatment of a disorder or condition selected from cancer, such as liver cancer (e.g. hepatocellular carcinoma) and lung cancer, which method comprises the administration of an effective amount of a compound of formula I, as hereinbefore defined.

Particular disorders or conditions that may be mentioned in relation to the aspects of the invention described hereinbefore include cancer, such as liver cancer (e.g. hepatocellular carcinoma) and lung cancer.

For the avoidance of doubt, in the context of the present invention, the term “treatment” includes references to therapeutic or palliative treatment of patients in need of such treatment, as well as to the prophylactic treatment and/or diagnosis of patients which are susceptible to the relevant disease states.

The terms “patient” and “patients” include references to mammalian (e.g. human) patients. As used herein the terms “subject” or “patient” are well-recognized in the art, and, are used interchangeably herein to refer to a mammal, including dog, cat, rat, mouse, monkey, cow, horse, goat, sheep, pig, camel, and, most preferably, a human. In some embodiments, the subject is a subject in need of treatment or a subject with a disease or disorder. However, in other embodiments, the subject can be a normal subject. The term does not denote a particular age or sex. Thus, adult and newborn subjects, whether male or female, are intended to be covered.

The term “effective amount” refers to an amount of a compound, which confers a therapeutic effect on the treated patient (e.g. sufficient to treat or prevent the disease). The effect may be objective (i.e. measurable by some test or marker) or subjective (i.e. the subject gives an indication of or feels an effect).

For the avoidance of doubt, references herein to compounds of formula I include, where the context permits, references to any of the specific compounds disclosed herein (whether falling within the scope of formula I or not). Further, references to any of compounds of formula I includes references to such compounds per se, to tautomers of such compounds, as well as to pharmaceutically acceptable salts or solvates, or pharmaceutically functional derivatives of such compounds.

Further embodiments of the invention that may be mentioned include those in which the compound of formula I is isotopically labelled. However, other, particular embodiments of the invention that may be mentioned include those in which the compound of formula I is not isotopically labelled.

The term “isotopically labelled”, when used herein includes references to compounds of formula I in which there is a non-natural isotope (or a non-natural distribution of isotopes) at one or more positions in the compound. References herein to “one or more positions in the compound” will be understood by those skilled in the art to refer to one or more of the atoms of the compound of formula I. Thus, the term “isotopically labelled” includes references to compounds of formula I that are isotopically enriched at one or more positions in the compound.

The isotopic labelling or enrichment of the compound of formula I may be with a radioactive or non-radioactive isotope of any of hydrogen, carbon, nitrogen, oxygen, sulfur, fluorine, chlorine, bromine and/or iodine. Particular isotopes that may be mentioned in this respect include ²H, ³H, ¹¹C, ¹³C, ¹⁴C, ¹³N, ¹⁵N, ¹⁵O, ¹⁷O, ¹⁸O, ³⁵S, ¹⁸F, ³⁷Cl, ⁷⁷Br, ⁸²Br and ¹²⁵I).

When the compound of formula I is labelled or enriched with a radioactive or nonradioactive isotope, compounds of formula I that may be mentioned include those in which at least one atom in the compound displays an isotopic distribution in which a radioactive or non-radioactive isotope of the atom in question is present in levels at least 10% (e.g. from 10% to 5000%, particularly from 50% to 1000% and more particularly from 100% to 500%) above the natural level of that radioactive or non-radioactive isotope.

Embodiments of the invention that may be mentioned include those in which the compounds of formula I provide selective anti-proliferative activity against SALL4-high (e.g. SNU398) cells as compared to SALL4-low (e.g. SNU387) cells.

When used herein in relation to anti-proliferative activity against SALL4-high (e.g. SNU398) cells as compared to SALL4-low (e.g. SNU387) cells, the terms “selective” and “selectivity” includes references to knockdown of SALL4-high cells at a concentration of compound of formula I that is at least 10-fold lower (e.g. at least 20-, 50-, 100-, 500- or 1000-fold lower) than that required for knockdown of SALL4-low cells at the same temperature (e.g. room temperature, such as 298 K).

Embodiments of the invention that may also be mentioned include those in which the compounds of formula I provide selective knockdown of SALL4-high cells.

In accordance with the invention, compounds of formula I may be administered alone (i.e. as a monotherapy, such as a monotherapy of a condition or disorder ameliorated by degradation of SALL4). In alternative embodiments of the invention, however, compounds of formula I may be administered in combination with another therapeutic agent (e.g. another therapeutic agent for the treatment of cancer).

Thus further aspects of the invention relate to the following.
(a) A compound of formula I, as hereinbefore defined, and another therapeutic agent for use in the treatment of a condition or disorder ameliorated by the degradation of SALL4 (e.g. SALL4B), such as cancer (e.g. lung cancer or liver cancer). In this aspect of the invention, the compound of formula I, as hereinbefore defined, may be administered sequentially, simultaneously or concomitantly with the other therapeutic agent.
(b) A compound of formula I, as hereinbefore defined, for use in the treatment of a condition or disorder ameliorated by the degradation of SALL4 (e.g. SALL4B), such as cancer (e.g. lung cancer or liver cancer), wherein the compound of formula I is administered sequentially, simultaneously or concomitantly with another therapeutic agent.
(c) Use of a compound of formula I, as hereinbefore defined, and another therapeutic agent for the preparation of a medicament for the treatment of a condition or disorder ameliorated by the degradation of SALL4 (e.g. SALL4B), such as cancer (e.g. lung cancer or liver cancer), wherein the compound of formula I is administered sequentially, simultaneously or concomitantly with the other therapeutic agent.
(d) Use of a compound of formula I, as hereinbefore defined, for the preparation of a medicament for the treatment of a condition or disorder ameliorated by the degradation of SALL4 (e.g. SALL4B), such as cancer (e.g. lung cancer or liver cancer), wherein the medicament is administered in combination with another therapeutic agent.
(e) A method of treatment of a disorder or condition ameliorated by the degradation of SALL4 (e.g. SALL4B), such as cancer (e.g. lung cancer or liver cancer), which method comprises the administration of an effective amount of a compound of formula I, as hereinbefore defined, and another therapeutic agent to a patient in need of such treatment.
(f) A combination product comprising
   (A) a compound of formula I, as hereinbefore defined, and
   (B) another therapeutic agent, wherein each of components (A) and (B) is formulated in admixture with a pharmaceutically-acceptable adjuvant, diluent or carrier.
(g) A combination product as defined at (f) above for use in the treatment of a condition or disorder ameliorated by the degradation of SALL4 (e.g. SALL4B), such as cancer (e.g. lung cancer or liver cancer).
(h) The use of a combination product as defined at (f) above for the manufacture of a medicament for the treatment of a condition or disorder ameliorated by the degradation of SALL4 (e.g. SALL4B), such as cancer (e.g. lung cancer or liver cancer).
(i) A method of treatment of a disorder or condition ameliorated by the degradation of SALL4 (e.g. SALL4B), such as cancer (e.g. lung cancer or liver cancer), which method comprises the administration of an effective amount of a combination product as defined at (f) above.

When used herein, the term “another therapeutic agent” includes references to one or more (e.g. one) therapeutic agents (e.g. one therapeutic agent) that are known to be useful for (e.g. that are known to be effective in) the treatment of cancer, e.g. lung cancer or liver cancer. The term “another therapeutic agent” may also include references to one or more (e.g. one) therapeutic agents (e.g. one therapeutic agent) that are known to be agonists of the E3 ligase cereblon (CRBN).

When used herein, the term “administered sequentially, simultaneously or concomitantly” includes references to:
administration of separate pharmaceutical formulations (one containing the compound of formula I and one or more others containing the one or more other therapeutic agents); and
administration of a single pharmaceutical formulation containing the compound of formula I and the other therapeutic agent(s).

The combination product described above provides for the administration of component (A) in conjunction with component (B), and may thus be presented either as separate formulations, wherein at least one of those formulations comprises component (A) and at least one comprises component (B), or may be presented (i.e. formulated) as a combined preparation (i.e. presented as a single formulation including component (A) and component (B)).

Thus, there is further provided:
(I) a pharmaceutical formulation including a compound of formula I, as hereinbefore defined and another therapeutic agent, in admixture with a pharmaceutically-acceptable adjuvant, diluent or carrier (which formulation is hereinafter referred to as a “combined preparation”); and
(II) a kit of parts comprising components:
   (i) a pharmaceutical formulation including a compound of formula I, as hereinbefore defined, in admixture with a pharmaceutically-acceptable adjuvant, diluent or carrier; and
   (ii) a pharmaceutical formulation including another therapeutic agent, in admixture with a pharmaceutically-acceptable adjuvant, diluent or carrier, which components (i) and (ii) are each provided in a form that is suitable for administration in conjunction with the other.

Component (i) of the kit of parts is thus component (A) in admixture with a pharmaceutically-acceptable adjuvant, diluent or carrier. Similarly, component (ii) is component (B) in admixture with a pharmaceutically-acceptable adjuvant, diluent or carrier.

Compounds of formula I may be administered by any suitable route, but may particularly be administered orally, intravenously, intramuscularly, cutaneously, subcutaneously, transmucosally (e.g. sublingually or buccally), rectally, transdermally, nasally, pulmonarily (e.g. tracheally or bronchially), topically, by any other parenteral route, in the form of a pharmaceutical preparation comprising the compound in a pharmaceutically acceptable dosage form. Particular modes of administration that may be mentioned include oral, intravenous, cutaneous, subcutaneous, nasal, intramuscular or intraperitoneal administration.

Compounds of formula I will generally be administered as a pharmaceutical formulation in admixture with a pharmaceutically acceptable adjuvant, diluent or carrier, which may be selected with due regard to the intended route of administration and standard pharmaceutical practice. Such pharmaceutically acceptable carriers may be chemically inert to the active compounds and may have no detrimental side effects or toxicity under the conditions of use. Suitable pharmaceutical formulations may be found in, for example, Remington The Science and Practice of Pharmacy, 19th ed., Mack Printing Company, Easton, Pennsylvania (1995). For parenteral administration, a parenterally acceptable aqueous solution may be employed, which is pyrogen free and has requisite pH, isotonicity, and stability. Suitable solutions will be well known to the skilled person, with numerous methods being described in the literature. A brief review of methods of drug delivery may also be found in e.g. Langer, Science (1990) 249, 1527.

Otherwise, the preparation of suitable formulations may be achieved routinely by the skilled person using routine techniques and/or in accordance with standard and/or accepted pharmaceutical practice.

The amount of compound of formula I in any pharmaceutical formulation used in accordance with the present invention will depend on various factors, such as the severity of the condition to be treated, the particular patient to be treated, as well as the compound(s) which is/are employed. In any event, the amount of compound of formula I in the formulation may be determined routinely by the skilled person.

For example, a solid oral composition such as a tablet or capsule may contain from 1 to 99% (w/w) active ingredient; from 0 to 99% (w/w) diluent or filler; from 0 to 20% (w/w) of a disintegrant; from 0 to 5% (w/w) of a lubricant; from 0 to 5% (w/w) of a flow aid; from 0 to 50% (w/w) of a granulating agent or binder; from 0 to 5% (w/w) of an antioxidant; and from 0 to 5% (w/w) of a pigment. A controlled release tablet may in addition contain from 0 to 90% (w/w) of a release-controlling polymer.

A parenteral formulation (such as a solution or suspension for injection or a solution for infusion) may contain from 1 to 50% (w/w) active ingredient; and from 50% (w/w) to 99% (w/w) of a liquid or semisolid carrier or vehicle (e.g. a solvent such as water); and 0-20% (w/w) of one or more other excipients such as buffering agents, antioxidants, suspension stabilisers, tonicity adjusting agents and preservatives.

Depending on the disorder, and the patient, to be treated, as well as the route of administration, compounds of formula I may be administered at varying therapeutically effective doses to a patient in need thereof.

However, the dose administered to a mammal, particularly a human, in the context of the present invention should be sufficient to effect a therapeutic response in the mammal over a reasonable timeframe. One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the potency of the specific compound, the age, condition, body weight, sex and response of the patient to be treated, and the stage/severity of the disease.

Administration may be continuous or intermittent (e.g. by bolus injection). The dosage may also be determined by the timing and frequency of administration. In the case of oral or parenteral administration the dosage can vary from about 0.01 mg to about 1000 mg per day of a compound of formula I.

In any event, the medical practitioner, or other skilled person, will be able to determine routinely the actual dosage, which will be most suitable for an individual patient. The above-mentioned dosages are exemplary of the average case; there can, of course, be individual instances where higher or lower dosage ranges are merited, and such are

The aspects of the invention described herein (e.g. the above-mentioned compounds, combinations, methods and uses) may have the advantage that, in the treatment of the conditions described herein, they may be more convenient for the physician and/or patient than, be more efficacious than, be less toxic than, have better selectivity over, have a broader range of activity than, be more potent than, produce fewer side effects than, or may have other useful pharmacological properties over, similar compounds, combinations, methods (treatments) or uses known in the prior art for use in the treatment of those conditions or otherwise.

Side effects that may be mentioned in this respect include side effects resulting from activity of therapeutic agents against non-cancerous cells. For example, since the SALL4 protein is generally not expressed by non-tumour cells, it is believed that the compounds of formula I will have negligible toxicity against non-cancerous cells. These benefits are demonstrated in vivo in the below Examples in which mice treated with a compound of formula I maintained normal bodyweight and spleen weight.

Compounds of formula I may be synthesised using known techniques as demonstrated in the below Examples. Other compounds may be prepared by analogous methods. Compounds of the invention may be isolated from their reaction mixtures using conventional techniques (e.g. recrystallisation, column chromatography, preparative HPLC, etc.).

In the processes described herein, the functional groups of intermediate compounds may need to be protected by protecting groups. The protection and deprotection of functional groups may take place before or after a reaction. Protecting groups may be removed in accordance with techniques that are well known to those skilled in the art.

The type of chemistry involved will dictate the need, and type, of protecting groups as well as the sequence for accomplishing the synthesis.

The use of protecting groups is fully described in “Protective Groups in Organic Chemistry”, edited by J W F McOmie, Plenum Press (1973), and “Protective Groups in Organic Synthesis”, 3^{rd} edition, T. W. Greene & P. G. M. Wutz, Wiley-Interscience (1999). As used herein, the term “functional groups” means, in the case of unprotected functional groups, hydroxy-, thiolo-, aminofunction, carboxylic acid and, in the case of protected functional groups, lower alkoxy, N-, O-, S-acetyl, carboxylic acid ester.

### EXAMPLES

### Materials

The drug library used in this study was from Novartis Institutes for Biomedical Research (NIBR), Cambridge, and consisted of 50,000 small molecules. The IMiDs used for comparison studies were Pomalidomide (>98% purity, Sigma-Aldrich), Lenalidomide (99.82% purity, Selleckchem, TX, USA), Thalidomide (>98% purity, Sigma-Aldrich), CC-122 (99.73% purity, Selleckchem, TX, USA) and CC-220 (98.67% purity, Selleckchem, TX, USA), and purity was assessed via NMR or HPLC. Sorafenib Tosylate (99.29% purity), Bortezomib (99.95% purity), MG132 (>97% purity), pevonedistat MLN4924 (99.01% purity) are from Selleckchem, TX, USA. QE-18-WG07 has a >95% purity from NIBR, Cambridge, USA, and >98% purity from Enamine.

Western blot primary antibodies, SALL4 EE30 (sc-101147), Beta-actin-HRP (sc-47778 HRP) and GAPDHHRP (sc-47724 HRP) were purchased from Santa Cruz Biotechnology. C-myc (D84C12), cleaved-PARP (D214) and Ikaros (IKZF1) (D10E5) was purchased from from Cell Signaling Technology. RBBP4/RbAp48 (NB100-60399), CRBN (NBP1-91810) and Aiolos (IKZF3) (NBP2-16938) were purchased from Novus Bio. The secondary antibodies, goat anti-mouse IgG-HRP (sc-2005 or sc-516102) and goat anti-rabbit IgG-HRP (sc-2004 or sc-2357), were purchased from Santa Cruz Biotechnology. Penicillin, streptomycin, L-glutamine, N-2 supplement (100×), B-27™ supplement (50×), Roswell Park Memorial Institute (RPMI) medium and 4-(2-hydroxyethyl)-1-piperazineethanesulfonic acid (HEPES) were purchased from Gibco, Waltham, MA. Fetal bovine serum (FBS) was purchased from Hyclone GE Healthcare, Chicago, IL. Advanced F12/Dulbecco's Modified Eagle Medium (DMEM) reduced serum medium (1:1) was purchased from Gibco, 12643. Bronchial Epithelial Cell Growth Medium with additives and MycoAlert detection kit were purchased from Lonza, Walkersville MD, USA. Epidermal growth factor and Immobilon Crescendo Western HRP substrate were purchased from Millipore, Billerica, MA. R-Spondin1 was purchased from R&D Systems, Minneapolis, MN. SB431542 was purchased from Tocris. Matrigel was purchased from Corning, Corning, NY. Isoflurane, USP was purchased from Baxter. CellTiter-Glo reagent (Cat #: G7573), Nano-Glo® Dual-Luciferase® Reporter Assay System (Cat #: N1650) and GoTaq qPCR Master Mix were purchased from Promega Corporation, Madison, WI. 10% neutral buffered formalin solution, Sypro Orange, Cremophor EL, Crystal Violet, Polybrene, Paraffin, Bovine Serum Albumin (BSA) were purchased from Sigma-Aldrich. DEN, PB, reducing sample buffer, Radio-Immune Precipitation Assay (RIPA) buffer, phenylmethylsulfonyl fluoride (PMSF) and protease inhibitor cocktail were purchased from Sigma, St. Louis, MO. Mini-PROTEAN Tetra Cell module, Mini Trans-Blot® module, Tris-Glycine Running buffer, Tris-buffered saline (0.1% Tween® 20 (TBST), polyvinylidene fluoride membrane (PVDF), polyacrylamide gel (8%) and Bradford Assay were purchased from Bio-Rad Laboratories. All reagents used in immunohistochemistry assay were from Dako (Dako, Glostrup, Denmark A/S). PBS was prepared from 137 mM NaCl, 2.7 mM KCl, 10 mM Na₂HPO₄, and 1.8 mM KH₂PO₄, purchased from Sigma-Aldrich. NP-40 lysis buffer was prepared from 10 mM Tris-HCl (pH 7.4), 150 mM NaCl, 2.7 mM KCl and 0.4% NP-40, purchased from Sigma-Aldrich. qPCR Lentivirus Titer Kit was purchased from abm (Cat #LV900). BD FACSAria™ was purchased from BD Biosciences. TRIzol® was purchased from Invitrogen. DNase I was purchased from Roche. RNeasy Mini kit was purchased from Qiagen. qScript® cDNA SuperMix was purchased from Quantabio. C-terminal FITC-labelled WT peptide (MSRRKQAKPQHI-FITC) was synthesised by Thermo Scientific. WT peptide (MSRRKQAKPQHI) and Scramble-peptide were purchased from Mimotopes, Australia. Mayer's Hematoxylin, 1.0% Eosin Y, 1.0% Phloxine B for Hematoxylin and Eosin (H&E) staining were purchased from VWR International.

### Cell Culture

All cell lines were obtained from ATCC, Manassas, VA. Human hepatocellular carcinoma (HCC) cell lines SNU-387, SNU-398, SNU-182, SNU-449, SNU-475, and non-small-cell lung carcinoma (NSCLC) cell lines H1299 and H661 were grown on standard tissue culture plates in filter-sterilized RPMI medium with 10% heat-inactivated FBS and 1% penicillin-streptomycin at 37° C. in a humidified atmosphere of 5% CO₂.

Cells were checked and confirmed to be mycoplasma-negative using a MycoAlert detection kit (Lonza).

### Analytical Techniques

### Sodium Dodecyl Sulfate Polyacrylamide Gel Electrophoresis (SDS-PAGE)

Protein samples were denatured by heating at 95° C. for 5 min in 1× reducing sample buffer. Appropriate volume of protein sample was loaded on the gel that has been assembled in the electrophoresis Mini-PROTEAN Tetra cell. Gels were ran in 1× Tris-Glycine Running buffer at 160 V for 45 min. After the electrophoresis run has completed, the power supply was turned off and the gels were removed from the cassettes for further analysis.

### RNA Extraction and Gene Expression Analysis

Cells were trypsinized, harvested and washed with cold PBS solution. Total RNA was extracted using TRIzol® as per manufacturer's instructions. Probable genomic DNA contamination was digested used DNase I. Subsequently, RNA was purified employing RNeasy Mini kit and used for cDNA synthesis utilizing qScript® cDNA SuperMix. For qRT-PCR, GoTaq qPCR Master Mix was used along with QuantStudio™ 5 Real-Time PCR System to detect and assess the RNA levels during thalidomide treatment, isoform knockdown and isoform overexpression. The qPCR primers used during this study are listed in Table 1 below.

TABLE 1
qPCR primers.

| qPCR PRIMER | | |
|---|---|---|
| SALL4TOTAL | FWD | GGTCCTCGAGCAGATCTTGT |
| SALL4TOTAL | REV | GGCATCCAGAGACAGACCTT |
| SALL4A | FWD | TCCTGGAAACCACATCCTTC |
| SALL4A | REV | ATGTGCCAGGAACTTCAACC |
| SALL4B | FWD | GGTGGATGTCAAACCCAAAG |
| 18SRNA | FWD | GTAACCCGTTGAACCCCATT |
| 18SRNA | REV | CCATCCAATCGGTAGTAGCG |
| ACTIN | FWD | ATGTGCAAGGCCGGCTTC |
| ACTIN | REV | TGACGATGCCGTGCTCGA T |

### Example 1

A new dual-luciferase SALL4 isoform-specific reporter cellular assay was designed and developed.

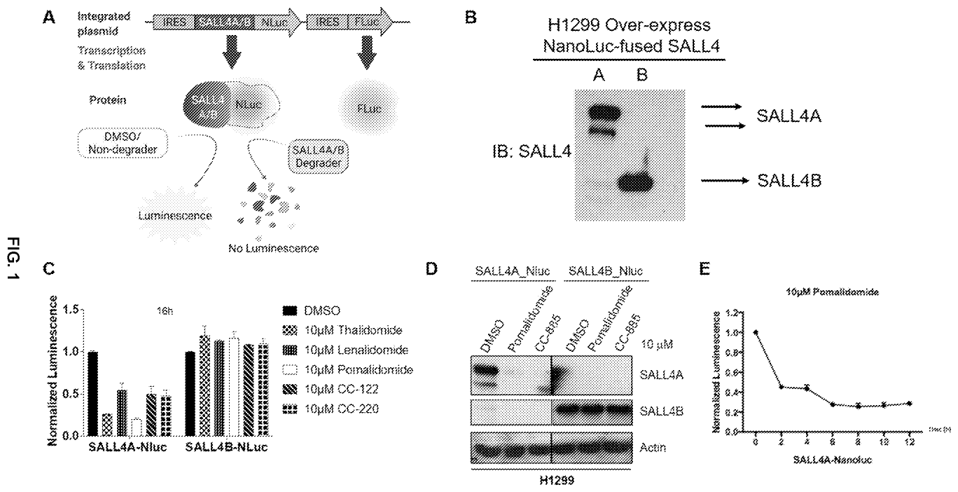

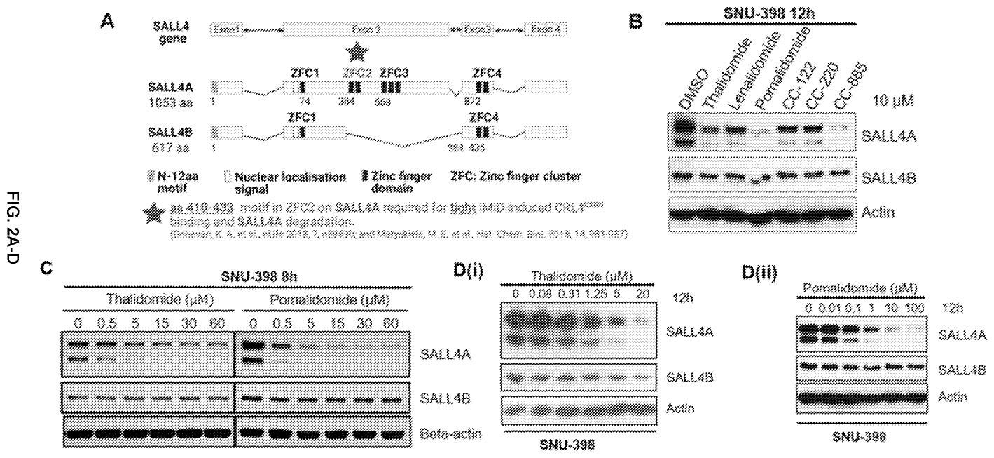

SALL4A or SALL4B was cloned into the dual-luciferase plasmid (pLL3.7-EF1a-IRES-Gateway-nluc-2xHA-IRES2-fluc-hCL1-P2A-Puro from Prof. William G. Kaelin group) to create a stable cell line (H1299, SALL4 negative) that ectopically express the SALL4A or B-NanoLuc fusion protein. SALL4A or B fused to nanoluciferase (Nluc) was expressed in a multicistronic mRNA with control firefly luciferase (Fluc). The measurement of Nluc/Fluc ratio enabled the determination of SALL4 fusion protein degradation relative to the internal Fluc control, independent of effects on transcription (FIG. 1A-B).

### Example 2

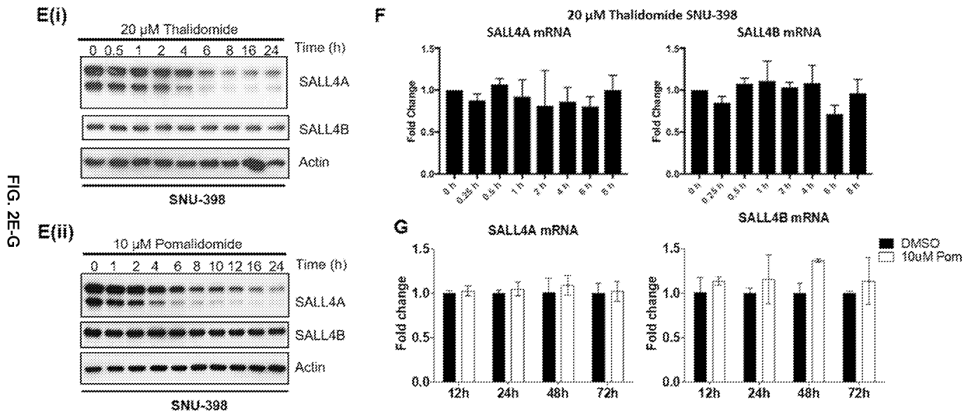

Recent reports identified SALL4 as the neosubstrate of IMiDs and demonstrated that IMiDs can degrade SALL4 (Donovan, K. A. et al., eLife 2018, 7, e38430; and Matyskiela, M. E. et al., Nat. Chem. Biol. 2018, 14, 981-987). Thus, the inventors tested whether IMiDs can be used to treat SALL4-mediated cancers. On the other hand, the effects of IMiDs treatment on SALL4 isoforms are undeciphered. The IMiDs-induced SALL4 degradation has been attributed to zinc finger domains (ZFC2 and 3, especially ZFC2) which are unique to SALL4A (Donovan, K. A. et al., eLife 2018, 7, e38430; and Matyskiela, M. E. et al., Nat. Chem. Biol. 2018, 14, 981-987) thus the inventors hypothesized that the SALL4B isoform which lacks ZFC2 and 3 would be resistant to iMIDs-induced degradation (FIG. 2A). To test for SALL4-isoform specific degradation by IMiDs, Thalidomide, Pomalidomide, Lenalidomide, CC-122 and CC-220 were taken for dual-luciferase SALL4 isoform-specific reporter cellular assay by following the protocol in Example 1.

### Results and Discussion

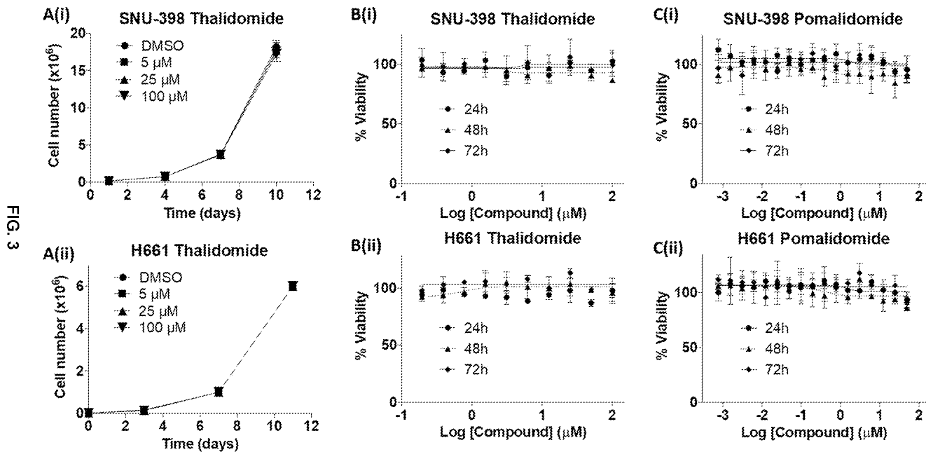

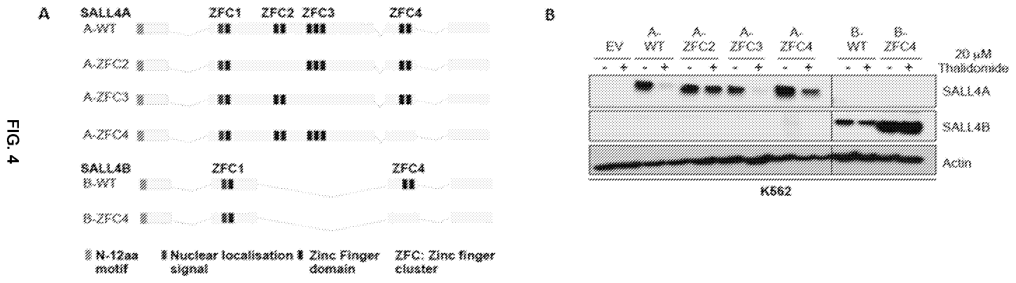

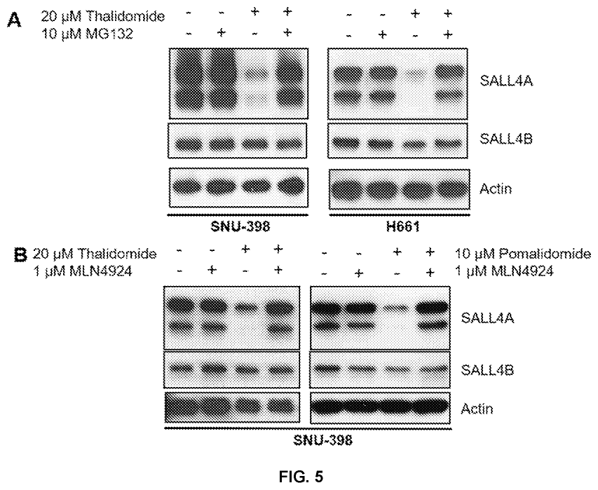

Surprisingly, the inventors found that SALL4-mediated cancer cells SNU-398 and H661 were insensitive to IMiDs treatment (FIG. 3). In the SALL4-isoform specific reporter system in H1299 cells (Example 1), it was observed that Thalidomide and its panel of derivatives selectively inhibited SALL4A-Nluc signal with different efficiency, while SALL4B-Nluc was not affected (FIG. 1C-D). Consistently, Thalidomide and its analogues showed dose-dependent SALL4A isoform specific degradation effect when tested in SNU-398 HCC cell line which expresses a high level of SALL4A and B endogenously (FIG. 2B-E), without affecting SALL4A or B mRNA transcript (FIG. 2F-G). Specific SALL4A degradation by IMiDs was further confirmed using the K562 cells overexpressing SALL4A or B (FIG. 4). Collectively, these observations suggest that SALL4A is not required for cancer cell viability and raise a possibility of the SALL4B dependency for SALL4-mediated cancer cell survival.

### Example 3

To study the kinetics of IMiDs-induced SALL4 isoforms degradation in the dual-luciferase SALL4 isoform-specific reporter system described in Example 1, the inventors treated the H1299 cells overexpressing SALL4A-nanoluc with 10 μM Pomalidomide at various time points.

### Results and Discussion

The rapid diminishing of SALL4A-Nluc signal was observed for the first 2 h (FIG. 1E). It was determined from FIG. 1D that the inhibition was time-dependent, which eventually became saturated at 8 h.

### Example 4

To find out if IMiDs-induced SALL4A degradation could be reversed in HCC SNU-398 and lung H661 cancer cells, the proteolytic activity of the 26S proteasome complex was inhibited with MG132 or neddylation was blocked with MLN4924.

### Inhibition of the Proteolytic Activity of the 26S Proteasome Complex and Blocking of Neddylation

26S proteasome complex and neddylation was inhibited by cotreating MG132 at 10 μM and MLN4924 at 1 μM for 6 h with 20 μM Thalidomide or 12 h with 10 μM Pomalidomide in SNU-398 or H661 cells. Cells were then lysed and subjected to further protein extraction and immunoblotting.

### Protein Extraction and Immunoblotting (Western Blot)

RIPA buffer containing freshly added protease inhibitors (2 mM PMSF) and protease inhibitor cocktail was used for cell lysis. The lysed cells were incubated for 10 min on ice and subsequently centrifuged at 4° C. for 10 min at 14,000 rpm. The protein concentrations of cell lysates were assessed using Bradford Assay as per manufacturer's instructions. The samples were denatured using 4× Sample buffer at 95° C. for 5 min. The samples were size resolved using polyacrylamide gel (8%) and Mini-PROTEAN Tetra Cell module. The size-resolved proteins were transferred onto PVDF via electroblotting using Mini Trans-Blot® module. The membrane was blocked using 5% BSA in TBST. The membrane was incubated with appropriate primary antibody (overnight) and secondary antibody (1 h). Immobilon Crescendo Western HRP substrate was used for signal development and images were acquired using darkroom development techniques.

### Results and Discussion

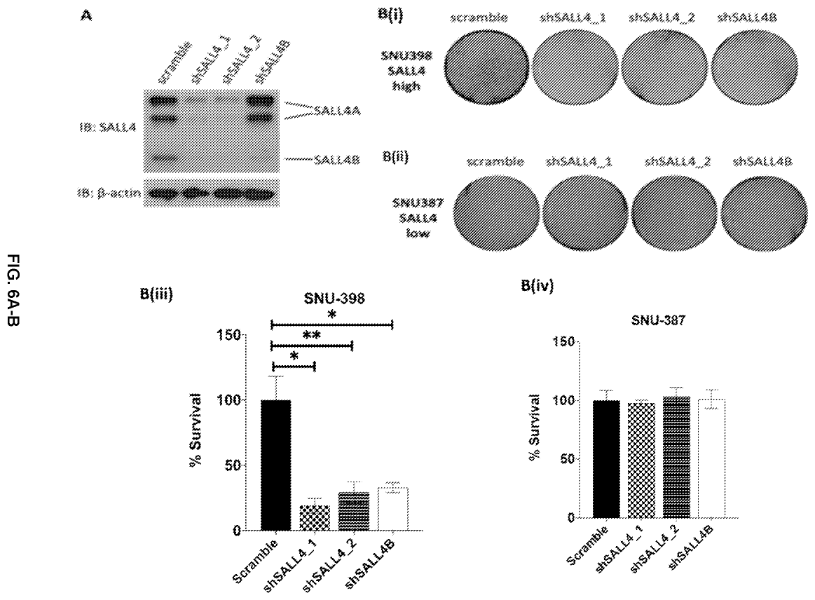

Indeed, by inhibiting the proteolytic activity of the 26S proteasome complex with MG132, or blocking neddylation with MLN4924, the IMiDs-induced SALL4A degradation could be reversed, suggesting that the SALL4A degradation by IMiDs works via the proteasomal system. Similar findings were also observed in SALL4-positive human H661 lung cancer cell line (FIG. 5).

Hence, combining the results obtained in Examples 1-4, the degradation of SALL4A by IMiDs has no anti-proliferative and therapeutic effects against SALL4-dependent cancers. This observation implies SALL4B's dependency on SALL4-mediated HCC and lung cancer oncogenesis and survival.

### Example 5

To investigate the importance of SALL4B for the survival of SALL4-positive HCC and lung cancer cells, shRNA against the unique region of SALL4B mature isoform mRNA was designed to attain SALL4B-specific silencing. The effect of SALL4B-specific knockdown on cancer cell apoptosis and survival was examined and compared to that of total SALL4 knockdown.

### SALL4 Knockdown by Lentiviral Transduction

shRNA targeting total SALL4 (sh-SALL4_1 & sh-SALL4_2)², SALL4B (sh-SALL4B) and a scrambled control (sh-scr)² with the following sequences were cloned into a pLL.3 vector containing GFP:

sh-scr:
5′-GGTACGGTCAGGCAGCTTCT-3′
sh-SALL4_1:
5′-GCTATTTAGCCAAAGGCAAA-3′
sh-SALL4_2:
5′-GCGTTGAAACAGGCCAAGCTG-3′
sh-SALL4B:
5′-CACAAGTGTCGGAGCAGTC-3′

For lentiviral transduction, SNU-398 cells were spin with lentiviral particles containing the aforementioned vectors at in RPMI (10% FBS, 5 μg/mL polybrene) at 800 g for 60 min at 32° C. Transduction efficiency was assessed via GFP signal using BD FACSAria™. The cells were then incubated at 37° C., 5% CO₂.

### Colony Formation Assay

SNU398, SNU-387, SNU-182, SNU-449, SNU-475, H661 and H1299 cells were trypsinized and counted 72 h after transduction. The cells were seeded at a dilution of 5,000-10,000 cells/well, and incubated for 7 days in a humidified incubator with 5% CO₂ at 37° C. The cells were fixed with 10% neutral buffered formalin solution and stained using Crystal Violet. The stained wells were washed with water until the excess dye was removed and left to dry overnight. The colonies were imaged and quantified using ImageJ.

### Soft Agar Assay

A base agar layer (0.6%) was prepared by diluting 1.2% agar with 2× complete culture media, and allowed to set in a 6-well plate. The cells were trypsinized post-treatment, counted, and diluted to a final concentration of 10,000 cells/well. A top agar layer (0.3%) containing a single-cell suspension was made by diluting 0.6% agar with the cell suspension prepared in the previous step. The cells were incubated in a humidified incubator with 5% CO₂ at 37° C. The culture medium was changed every two days. The colonies were imaged and quantified using ImageJ.

### Cell Titre Glo Viability Assay

Cells were plated in RPMI culture media (40 μL) in 384-well white, flat-bottom plates (Corning) and incubated at 37° C. in a humidified atmosphere of 5% CO₂ overnight. The numbers of cells per well were 1,500 for SNU-398 and SNU-182, 750 for SNU-387, SNU-449, SNU-475 and SNU-423, and 1,000 for H661 and H1299. After overnight incubation, 10 μL of drug (1% DMSO in RPMI) at designed concentrations were added to the cells. The cells were then incubated for 16, 24, 48, 72, or 96 h at 37° C. in a humidified atmosphere of 5% CO₂ before CellTiter-Glo reagent (10 μL) was dispensed to the wells. The cells were incubated at room temperature for a minimum of 10 min. After that, luminescence readings were recorded by a Microplate Reader (PHERAstar or Tecan Infinite M200 Pro) according to the manufacturer's instruction for measuring the luminescence.

### Caspase Glo Assay

The assay was performed in a 384-well plate (Corning 3570). Cells were seeded at 5,000 cells per well in 30 μL of RPMI medium and incubated overnight at 37° C., 5% CO₂. 10 μL of compounds or DMSO vehicle in RPMI (0.5% final DMSO) was added. After 20 h or 24 h of incubation, Caspase-glo reagent (30 μL) was added and the plate was allowed to sit at room temperature in the dark for 2 h. The luminescence was monitored and recorded using Tecan Infinite M200 Pro plate reader.

### Results and Discussion

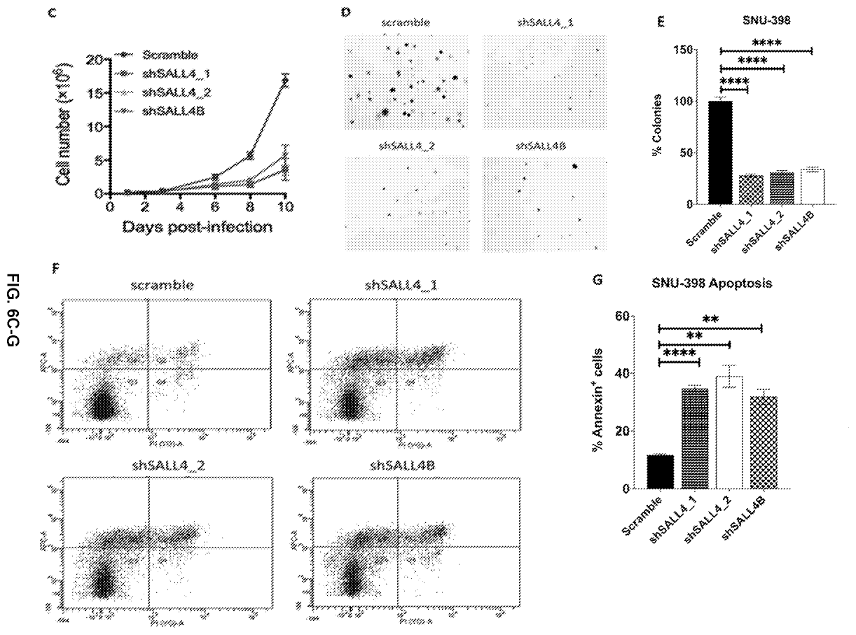

To test the dependency of cancer cell survival on SALL4B as compared to total SALL4, colony formation assay was performed post-knockdown of total SALL4 or SALL4B in a panel of SALL4-high HCC (SNU-398, SNU-182) and lung cancer (H661) cells. To rule out SALL4-independent off-target cytotoxicity effects of the shRNA, a panel of SALL4-low HCC (SNU-387, SNU-475, SNU-449) and lung cancer (H1299) cells were treated with the same aforementioned shRNA, followed by colony formation assay. It was observed that both total SALL4 and SALL4B-specific knockdown significantly decreased the number of colonies in the panel of SALL4-high HCC and lung cancer cells to the same extent as compared to the scramble control cells. In contrast, SALL4-low cancer cell growth was unaffected by total SALL4 or SALL4B-specific knockdown (FIG. 6A-B and 7). These results suggest that SALL4B is essential for the differentiation and proliferation of these SALL4-high HCC and lung cancer cells.

Since HCC is the focused disease model, the inventors further confirmed the anticancer effect of SALL4B silencing observed from the colony formation assay in both soft agar and cell viability assays by comparing the number of viable cells after total SALL4 and SALL4B-specific knockdown. Both of these assays showed a similar trend that, relative to total SALL4 knockdown, SALL4B silencing alone caused comparably significant inhibition of SNU-398 cell viability (FIG. 6C) and suppression of survival and anchorage-independent growth (FIG. 6D) of SALL4-positive HCC Cell (SNU-398).

Additionally, in SALL4-high HCC SNU-398 cells, SALL4B knockdown enhanced apoptosis by 40% (Annexin V staining), similar to that of a total SALL4 knockdown (FIG. 6F-G). This further suggests SALL4B as the critical isoform in conferring a survival advantage to SALL4-mediated cancer cells.

Together, these data demonstrate SALL4B as the critical isoform in conferring a survival advantage to SALL4-mediated cancer cells.

### Example 6

To validate the role of SALL4B in the initiation and maintenance of HCC in vivo, the inventors utilized the reported SALL4B transgenic mice (Ma, Y. et al., Blood 2006, 108, 2726-2735). The inventors utilized genetically engineered mouse models expressing human SALL4B isoform in liver post-birth. These mice were challenged with the two-stage chemical carcinogenesis protocol to induce liver tumors in mice.

### SALL4B Transgenic Mice and Two-Stage Chemical Carcinogenesis

SALL4B transgenic mice were generated at the background of C57BL/6 as previously described (Ma, Y. et al., Blood 2006, 108, 2726-2735), and were maintained at the mouse facility at Children's Hospital Boston (CHB). All animal work was approved by the IACUC under protocol 10-10-1832. The SALL4B primer sequences for genotyping include the following: forward primer, 5′-AGC AGA GCT CGT TTA GTG AAC CG-3′, and reverse primer, 5′-CTG TCA TTC ATG ATG AGG ACA GG-3′. For the two-stage chemical carcinogenesis experiment, both SALL4B transgenic and wild type C57BL/6 mice received a single intraperitoneal injection (IP) of 5 mg DEN/kg body weight in sterile water at 15 days of age. Mice were then administered 0.05% of PB in drinking water from 2 weeks after DEN injection (4 weeks of age). Mice were sacrificed 10 and 23 weeks after the start of the PB diet by carbon dioxide inhalation and necropsied. Deaths and moribund cases were also necropsied. Body weights were recorded and livers removed, weighed and examined for grossly visible lesions. Each liver lobe was fixed in 10% neutral buffered formalin, trimmed, and embedded in paraffin. For routine histological analysis, two representative sections were prepared from each liver lobe, 4-6 μm sections were prepared from paraffin blocks and stained with H&E. Liver lesions were classified according to criteria defined previously. The present of foci of cellular alteration was graded based on the percentage of foci area compared to the liver section as following: +: <15%, ++: 15-40%, +++: 40-75%, ++++: 75-100%.

### Results and Discussion

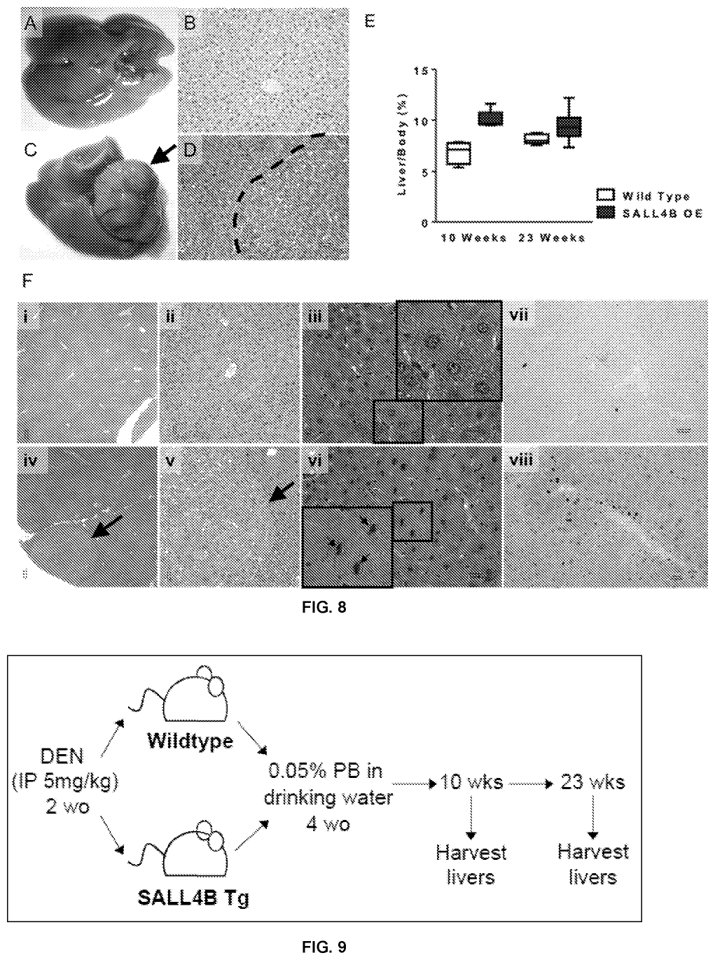

Remarkably, the monitoring of transgenic mice over time revealed an increased risk of liver tumor formation in SALL4B transgenic mice from 16 months onwards. No evidence of tumor formation was detected in the livers of the 19 age-and sex-matched wild type (WT) controls (FIG. 8A-B and Table 2). The inventors observed liver tumor formation in 50% ( 7/14) of the SALL4B transgenic mice (FIG. 8C-D). This observation indicates that SALL4B plays a significant role in driving in vivo hepatocellular oncogenesis.

TABLE 2
Summary of liver tumor incidences in SALL4B transgenic mice.

| | Macroscopic tumor | Microscopic tumor/lesion |
|---|---|---|
| Genotyping | incidence | incidence |
| SALL4B | 4/14 | 3/14 |
| WT | 0/19 | 0/19 |

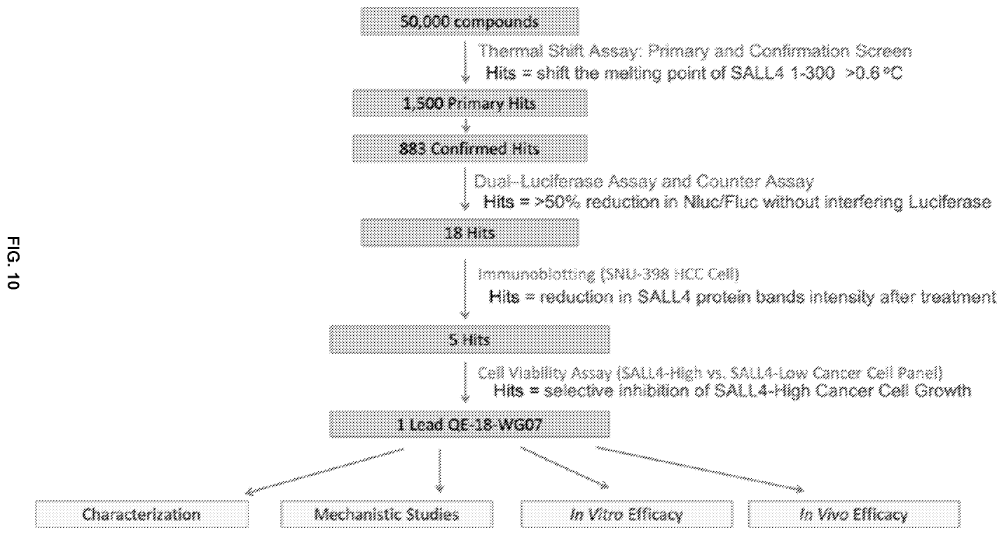

To further confirm that young SALL4B transgenic mice are more susceptible to liver tumor development, the inventors employed the two-stage chemical carcinogenesis protocol using DEN and PB to induce pre-neoplastic lesions in the transgenic livers. Mice were examined 10 weeks and 23 weeks post-PB treatment (FIG. 9). Consistently, young SALL4B transgenic mice exhibited a greater risk of hepatic lesions development (FIG. 8F (iv-vi) and Table 3) and, later, hepatic tumor formation (FIG. 8F (viii) and Table 4) upon carcinogens exposure. These findings support that SALL4B is critical for in vivo hepatocarcinogenesis.

TABLE 3
Summary of liver lesions in SALL4B transgenic mice subjected
to the DEN initiation and PB promotion regime.

| | | Experimental | Pre-neoplastic | Macroscopic | Liver | Liver/body |
|---|---|---|---|---|---|---|
| Mouse ID | Genotype | week* | foci^{†} | tumor | weight (g) | weight |
| 492150-1 | WT | 10 | − | − | 1.23 | |
| 492150-2 | WT | 10 | − | − | 1.55 | |
| 492150-3 | WT | 10 | − | − | 1.80 | |
| 492150-4 | WT | 10 | − | − | 1.70 | |
| 492156-1 | WT | 10 | − | − | 1.99 | |
| 492156-2 | WT | 10 | − | − | 1.72 | |
| 492156-3 | WT | 10 | − | − | 2.11 | |
| Average | | | | | 1.73 | 7.1% |
| 492152-R | SALL4B | 10 | ++ | − | 2.31 | |
| 492152-L | SALL4B | 10 | ++ | − | 2.37 | |
| 492152-RL | SALL4B | 10 | ++ | − | 2.32 | |
| 492152-RR | SALL4B | 10 | ++ | − | 2.28 | |
| 492152-LL | SALL4B | 10 | ++ | − | 2.79 | |
| Average | | | | | 2.41 | 9.3% |
| 492146-1 | WT | 23 | + | − | 2.40 | |
| 492146-2 | WT | 23 | + | − | 2.27 | |
| 492146-3 | WT | 23 | + | − | 2.41 | |
| 492146-4 | WT | 23 | + | − | 2.64 | |
| 492146-5 | WT | 23 | + | − | 2.56 | |
| Average | | | | | 2.46 | 6.7% |
| 492148-R | SALL4B | 23 | ++ | − | 2.83 | |
| 492148-L | SALL4B | 23 | +++ | − | 2.28 | |
| 492149-RL | SALL4B | 23 | +++ | − | 2.75 | |
| 492149-RR | SALL4B | 23 | +++ | − | 2.97 | |
| 492149-LL | SALL4B | 23 | +++ | + | 3.79 | |
| 492149-NO | SALL4B | 23 | ++ | − | 2.94 | |
| Average | | | | | 2.93 | 9.4% |

*Mice were 15 days of age at start of experiment. Mice were given 5 mg/kg DEN IP at 15 days of age and PB diet was started at 4 weeks of age.
^{†}The present of hyper proliferative foci was graded based on the percentage of foci area compared to the liver section: +: <15%, ++: 15-40%, +++: 40-75%, ++++: 75-100%.

TABLE 4
Ki-67 expression in DEN/PB-treated mouse livers.

| | | | | | Mitotic |
|---|---|---|---|---|---|
| | | Experimental | Strong Ki-67 | Strong positive | cells/10x |
| Mouse ID | Genotype | week* | cells/10x field | foci/10x field | field |
| WT1 | Wild type | 10 | <5 | <2 | 0 |
| WT2 | Wild type | 10 | <5 | <2 | 0 |
| WT3 | Wild type | 10 | <5 | <2 | 0 |
| WT4 | Wild type | 10 | <5 | <2 | 0 |
| WT5 | Wild type | 10 | <5 | <2 | 0 |
| WT6 | Wild type | 23 | 5-10 | <2 | 0 |
| WT7 | Wild type | 23 | 5-10 | <2 | 0 |
| WT8 | Wild type | 23 | 5-10 | <2 | 0 |
| WT9 | Wild type | 23 | 5-10 | <2 | 0 |
| WT10 | Wild type | 23 | 5-10 | <2 | 0 |
| S4B1 | SALL4B | 10 | >10 | 1-5 | 0 |
| S4B2 | SALL4B | 10 | >10 | 1-5 | 0 |
| S4B3 | SALL4B | 10 | >10 | 1-5 | 0 |
| S4B4 | SALL4B | 10 | >10 | 1-5 | 0 |
| S4B5 | SALL4B | 10 | >10 | 1-5 | 0 |
| S4B6 | SALL4B | 23 | >50 | 1-5 | 1-5 |
| S4B7 | SALL4B | 23 | >50 | 1-5 | 1-5 |
| S4B8 | SALL4B | 23 | >50 | 1-5 | 1-5 |
| S4B9 | SALL4B | 23 | >50 | 1-5 | 1-5 |
| S4B10 | SALL4B | 23 | >50 | 1-5 | 1-5 |

*Mice were 15 days of age at start of experiment. Mice were given 5 mg/kg DEN IP at 15 days of age and PB diet was started at 4 weeks of age.

### Example 7

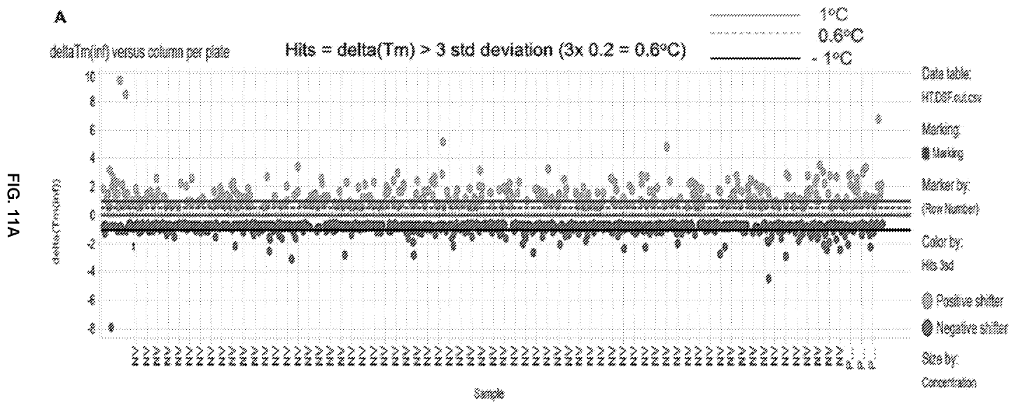

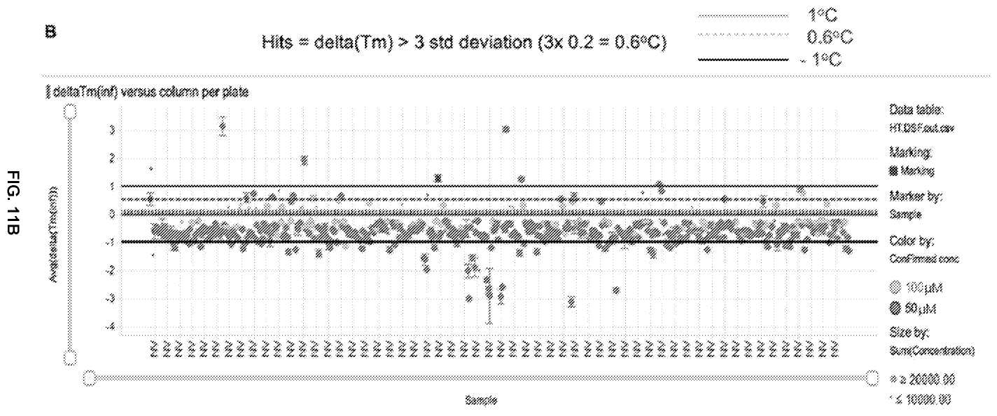

With the knowledge that SALL4B is important for HCC tumorigenesis and that the current therapy only targets SALL4A but not SALL4B, the inventors developed a novel high throughput screening (HTS) platform that encompasses an assay cascade, including thermal shift assay (TSA), the newly developed dual-luciferase SALL4 protein level reporter cellular assay in Example 1, and cell viability assay in Example 5, to identify potential SALL4 binders and degraders that can inhibit SALL4-positive cancer cell survival. A summary of the screening workflow and hits selection is described below and in FIG. 10.

### Production of SALL4 (1-300aa) Protein in E. Coli for HTS

DH5alpha were transformed with pET His6 TEV LIC Cloning vector (2B-T) containing SALL4 (1-300aa). pET His6 TEV LIC cloning vector (2B-T) was a gift from Scott Gradia (Addgene plasmid #29666). SALL4 (1-300aa) cloned into this vector was expressed with the N-terminal 6-His tags in the E. coli BL21 (DE3)/pLysS strain using a T7 expression system. SALL4 1-300 was purified by His trap Ni²⁺ column and gel filtration. After purification, the SALL4 (1-300) protein was further characterized by SDS-PAGE Gel for purity and check of identity. Functional assay (fluorescent polarity assay) to confirm the produced SALL4 (1-300aa) ability to form complex with RBBP4 protein partner. Thermal shift assay was used to evaluate the protein's thermal stability. The yield of protein obtained from 1 L of E. coli culture was 6 mg. A total of 686 mg of SALL4 1-300 protein was produced for drug screening and characterization assays.

### Fluorescence Polarization to Confirm SALL4 (1-300) Function

SALL4 (1-300aa) was serially-diluted to obtain 10 concentration points. Each diluted SALL4 (1-300aa) (2 μL) was then titrated into a 384-well plate (flat μClear® bottom, non-binding, black, Greiner microplate) at 10× concentration. 18 μL of a reaction mixture of RBBP4 and C-terminal FITC-labelled WT peptide (MSRRKQAKPQHI-FITC, 1 μM) was added to each well containing 2 μL of SALL4 (1-300aa) or controls, and incubated at room temperature for 2 h. The final reaction mixture contained RBBP4 (0.25 μM), C-terminal FITC-labelled WT peptide (MSRRKQAKPQHI-FITC, 1 μM), and SALL4 (1-300aa) (0.0195 μM to 10 μM in 20 mM Tris, pH 8). The controls tested were WT peptide (MSRRKQAKPQHI, 5 μM), and Scramble-peptide (5 μM).

### Thermal Shift Assay (TSA) for HTS—Primary Screen

A library of 50,000 small molecules from the Novartis Biomedical Research Institute (NIBR) was screened at 50 μM and 100 μM against 13.8 μM of purified SALL4 (1-300aa) protein in the buffer containing potassium phosphate (100 mM) and NaCl (100 mM) at pH 7. Screening assay was performed in 384-well thin wall white PCR plates using LightCyler® 480 Real-Time PCR System (Roche Applied Sciences). A 7-min run was started at 30° C. and ended at 90° C. Sypro Orange was used as the sensitive fluorescent dye for monitoring thermal protein denaturation. Hits were defined as compounds that can induce a change in protein melting points 3 times higher than the standard deviations of the apo-protein melting point from DMSO control.

### Dual-Luciferase Assay for HTS—Secondary Screen

The assay was performed as described in Example 1 except in a 1,536-well plate which allows HTS of thermal shift hits from the primary screen, searching for SALL4 small molecule binders that can degrade the oncogenic SALL4B isoform. H1299 cells that stably express SALL4A/B_Nluc_Fluc were seeded in a 1,536-well clear bottom white plate (Corning) at 2,000 cells per well in RPMI medium (6 μL) and grown overnight (16 h) at 37° C., 5% CO₂. Cells were treated with 8 μM of compounds for 6 h. Nano-Glo® Dual-Luciferase® Reporter Assay System and Pherastar plate reader were used for measuring the luciferase signal, following the manufacturer's instruction. For data analysis, the Nanoluc luminescence is normalized to firefly luciferase reading and then compared to the DMSO control. The screen was performed in triplicates.

A counter-screen was also performed to eliminate false positive hits that interfere with the luciferase signal. The protocol for the counter screen is similar to that of the main screen, except that there was no incubation period of 6 h. Hits were selected as compounds that can result in 50% or more reduction in Nluc/Fluc signal in the main screen with minimal interference (<15% signal reduction) of luciferase signal itself in the counter-screen.

The isoform-focused screening platform is robust, quantifiable, sensitive, and reproducible. Thus, it could serve as a valuable tool for rapidly identifying compounds capable of chemically knocking down new pathological protein isoforms in other diseases.

### Example 8

The inventors initiated a HTS on the drug library by following the protocol in Example 7 to discover compounds that can target and degrade the SALL4B isoform for the treatment of SALL4-mediated HCC. Confirmed hits were further subjected to western blotting and cell viability assay for validation by following the protocols in Examples 4 and 5, respectively.

### Results and Discussion

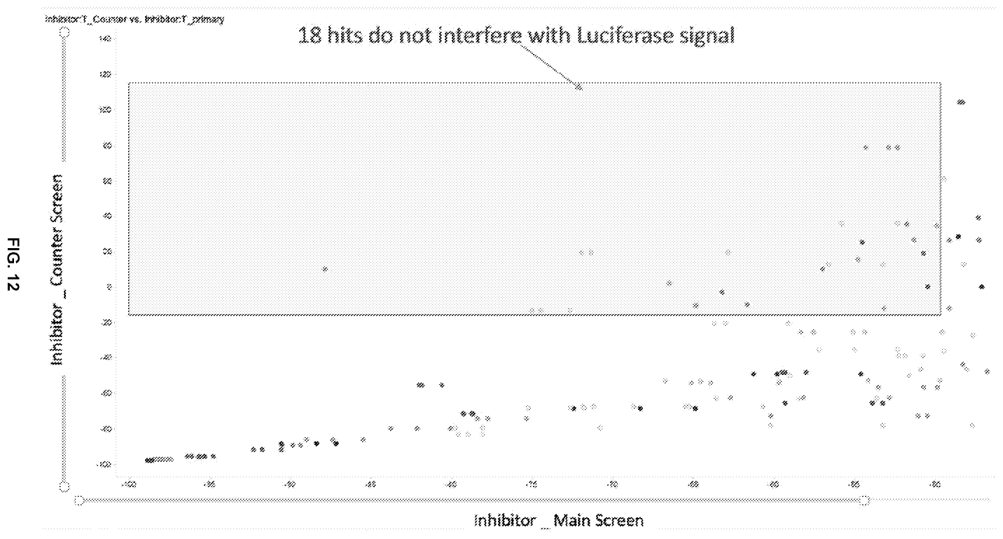

For the development of targeted SALL4 degrader, TSA was first utilized as the in-solution direct-binding assay in the primary screen to discover strong binders of SALL4 with binding affinity <10 μM. This assay assesses the binding of small molecules to protein based on changes in unfolding transition, which is monitored via a fluorescent reporter ligand. Sypro-orange was used as the reporter dye in the screen. A binder or hit was identified when there is a shift in the melting point of the protein in the presence of the compound as compared to the melting point of apo-protein (SALL4 (1-300aa)) in the DMSO control. The inventors set the hits cut-off as any compounds that induce a ≥0.6° C. change in the melting point of SALL4 (1-300aa), which is within 95% confidence interval or 3 standard deviations (s.d.) of the melting point variation of apo-protein (0.2° C., FIG. 11A). After screening 50,000 compounds at a single concentration of 100 μM, 1500 primary SALL4 (1-300aa) binder hits were obtained. These hits were further subjected to TSA confirmation screen in duplicates and at two concentrations, 50 μM and 100 μM. Confirmed hits were defined as compounds with >3 s.d. shift in protein melting temperature (ΔTₘ) at either 100 or 50 μM with no negative error codes, and gave ˜146 positive shifters and ˜737 negative shifters. Compounds that were originally scored as positive shifters but were later validated as negative shifters or vice versa in the confirmation screen were not taken. As compared to 100 μM, compounds dosed at 50 μM appeared to have a bigger shift (FIG. 11B). This could be due to the solubility limitation of these compounds at a high concentration of 100 μM, which caused the compounds to precipitate out of the solution and hence, they displayed lower observed activity in the assay.

A total of 883 TSA hits, which were potential SALL4 binders, were then tested for their ability to induce SALL4B isoform degradation in the dual-luciferase cellular assay in which SALL4B has been cloned into the dual-luciferase plasmid. A drug that induces SALL4B degradation will result in a decrease or loss of luciferase signal (Fluc/Nluc) as compared to the vehicle (DMSO) control (FIG. 1A). To eliminate false-positive artefacts due to luciferase interference, the 883 hits were also simultaneously subjected to counter-screening. 18 compounds were identified to be true hits in the luciferase reporter assay that caused a loss of ≥45% in Fluc/Nluc signal after 6 h of treatment at 8 μM (FIG. 12).

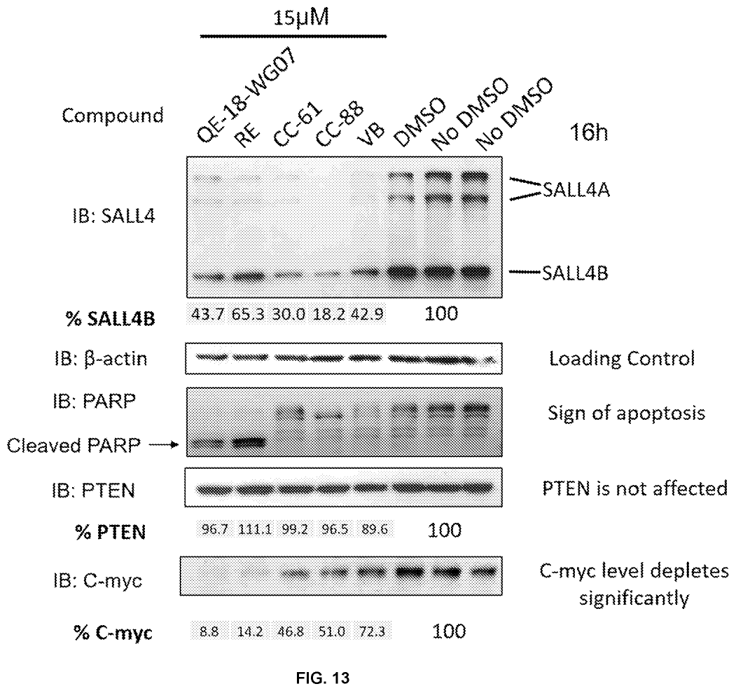

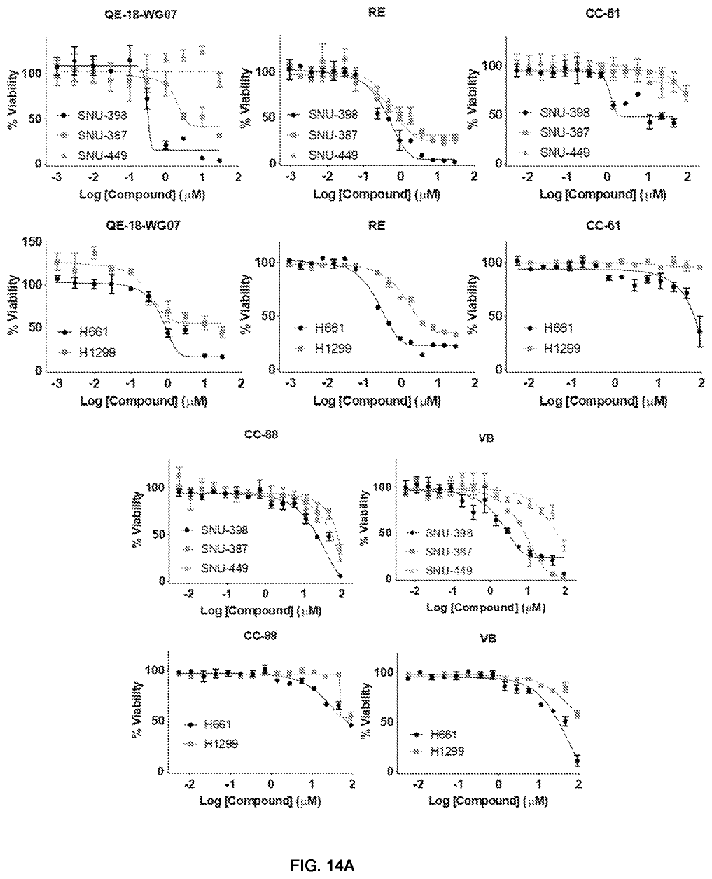

Since the H1299 cells used in the dual-luciferase cell assay were artificially created to over-express SALL4B protein that fused with the luciferase reporter for HTS, there is a need to assess if the hits in this reporter assay indeed induced targeted SALL4 degradation in a HCC cancer cell line with endogenous aberrant SALL4 expression. Therefore, as a follow-up study, endogenously SALL4-high HCC Cell Line SNU-398 was incubated with the 18 hits from the dual-luciferase cell assay at 15 μM for 16 h, lysed and the cell lysates after drug treatment were subjected to western blotting. It was observed that 5 out of 18 hits indeed induced degradation of the endogenous SALL4B protein (34.7 to 81.8%) in SNU-398 HCC cells at 15 μM after 16 h of treatment (FIG. 13). The immunoblot also revealed that these 5 hits also decreased the protein expression of the SALL4A isoform. The average temperature shift ΔTₘ in TSA and the percentage of Fluc/Nluc signal loss in dual-luciferase cell assay induced by these 5 validated hits are summarized in Table 5.

TABLE 5
Summarized Data from TSA and dual-luciferase cell assay
of the 5 hits validated in immunoblot (FIG. 13).

| | Thermal Shift Assay | | SALL4B_Luc Reporter Assay |
|---|---|---|---|
| Compound | Type | ΔTm (° C.)* | % SALL4B Downregulation^{†} |
| QE | Positive shifter | 1.15 ± 0.01 | 29.8 ± 1.8 |
| RE | Negative shifter | 0.56 ± 0.08 | 9.2 ± 2.0 |
| CC-61 | Positive shifter | 1.52 ± 0.\|07 | 56.1 ± 2.4 |
| CC-88 | Negative shifter | 0.58 ± 0.04 | 16.6 ± 2.9 |
| VB | Positive shifter | 0.50 ± 0.20 | 53.2 ± 1.0 |

*SALL4(1-300) melting temperature shift, mean ± SD (n = 2). [compound] = 100 μM, 30 min.
^{†}Normalized to DMSO control, mean ± SD (n = 3). [compound] = 8 μM, 6 h treatment, H1299 cells.

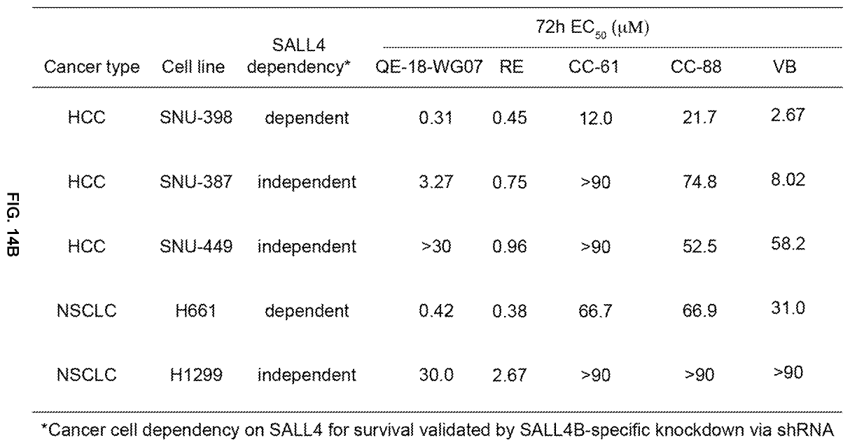

Next, the inventors aimed to select small molecule degraders that have on-target anti-cancer activity against SALL4-high expressing cancer cells. By screening the 5 best SALL4 degrader hits from the immunoblot in cell viability assay consisting of a panel of both SALL4-high and low HCC and lung cancer cell lines, a compound with the best potency (low IC₅₀ value) and selectivity against SALL4-high cancer cells was selected as the lead compound. Among the 5 immunoblot hits, QE-18-WG07 induced the most potent and selective anti-proliferative effects against SALL4-high cancer cells over SALL4-low cancer cells (up to 58-fold selectivity), with half maximal growth inhibition EC₅₀=310 nM and 420 nM in SNU-398 and H661 cells, respectively (FIG. 14). Therefore, QE-18-WG07 was chosen as the lead SALL4 degrader candidate for further mechanistic investigation, in vitro and in vivo anti-tumor activity evaluation studies.

### Example 9

The potency of 35 compounds from the drug library was compared to QE-18-WG07 by following the HTS dual-luciferase cellular assay protocol in Example 7.

### Results and Discussion

The main screen readout for QE-18-WG07 was −31, hence it induced a 31% reduction in the amount of SALL4B protein after 6 h of incubation in H1299 cells overexpressing SALL4B fused with Nluc. Synthesised compounds are identified in Table 6A below, while activity data is provided in Table 6B.

TABLE 6A
Identification of the synthesized compounds.

| Identifier | Structure | MW | ALogP | LogD | PSA |
|---|---|---|---|---|---|
| XF-84-GG52 | 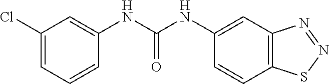 [SMILES: O=C(Nc1cccc(Cl)c1)Nc1ccc2snnc2c1] | 304.75476 | 3.36 | 3.36 | 95.15 |
| KE-95-GC86 | 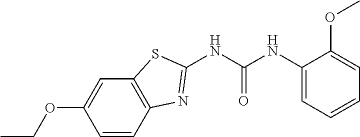 [SMILES: CCOc1ccc2nc(NC(=O)Nc3ccccc3OC)sc2c1] | 343.40018 | 3.464 | 3.485 | 100.72 |
| DE-94-ST40 | 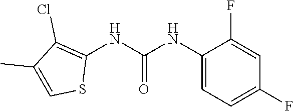 [SMILES: Cc1csc(NC(=O)Nc2ccc(F)cc2F)c1Cl] | 302.72746 | 3.871 | 3.871 | 69.36 |
| BC-46-HY47 | 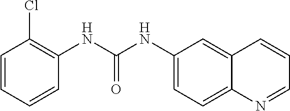 [SMILES: O=C(Nc1ccc2ncccc2c1)Nc1ccccc1Cl] | 297.73898 | 3.373 | 3.373 | 54.02 |
| QE-95-GV86 | 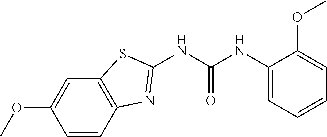 | 329.37359 | 3.115 | 3.136 | 100.72 |
| RB-06-II47 | 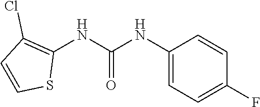 [SMILES: O=C(Nc1ccc(F)cc1)Nc1sccc1Cl] | 270.71042 | 3.179 | 3.179 | 69.37 |
| NA-78-BR04 | 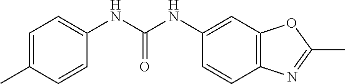 | 281.30919 | 2.795 | 2.795 | 67.16 |
| NF-48-LK42 | 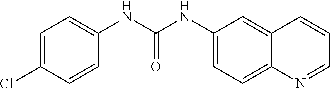 [SMILES: O=C(Nc1ccc(Cl)cc1)Nc1ccc2ncccc2c1] | 297.73898 | 3.373 | 3.383 | 54.02 |
| HE-94-SP40 | 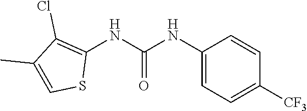 [SMILES: Cc1csc(NC(=O)Nc2ccc(C(F)(F)F)cc2)c1Cl] | 334.7445 | 4.402 | 4.402 | 69.36 |
| AE-96-GL86 | 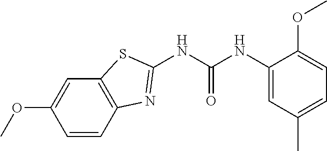 | 343.40018 | 3.601 | 3.622 | 100.72 |
| QE-18-WG07 (QE) | 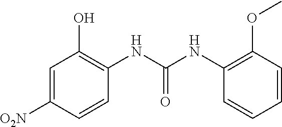 [SMILES: COc1ccccc1NC(=O)Nc1ccc([N+](=O)[O-])cc1O] | 303.27011 | 2.158 | 1.917 | 116.41 |
| ZB-04-ZW40 | 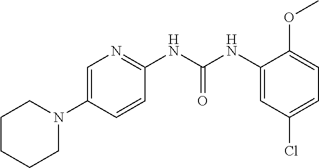 [SMILES: COc1ccc(Cl)cc1NC(=O)Nc1ccc(N2CCCCC2)cn1] | 359.84987 | 4.25 | 3.988 | 53.6 |
| TD-57-GF27 | 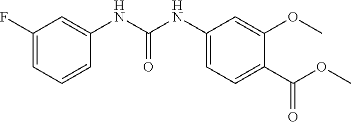 [SMILES: COC(=O)c1ccc(NC(=O)Nc2cccc(F)c2)cc1OC] | 318.2997 | 2.567 | 2.567 | 76.66 |
| RD-10-AY51 | 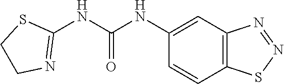 [SMILES: O=C(NC1=NCCS1)Nc1ccc2snnc2c1] | 279.34135 | 2.184 | 2.097 | 132.81 |
| QB-77-CJ52 | 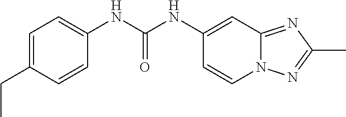 [SMILES: CCc1ccc(NC(=O)Nc2ccn3nc(C)nc3c2)cc1] | 295.33907 | 2.216 | 2.216 | 71.32 |
| DC-23-NP48 | 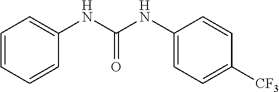 [SMILES: O=C(Nc1ccccc1)Nc1ccc(C(F)(F)F)cc1] | 281.2332 | 2.853 | 2.987 | 54.02 |
| PB-97-IJ47 | 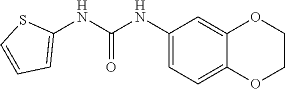 [SMILES: O=C(Nc1ccc2c(c1)OCCO2)Nc1cccs1] | 276.31098 | 2.095 | 2.095 | 87.83 |
| VE-35-SA30 | 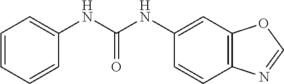 [SMILES: O=C(Nc1ccccc1)Nc1ccc2ncoc2c1] | 253.25604 | 2.048 | 2.048 | 67.16 |
| ND-41-RW44 | 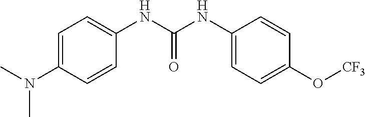 [SMILES: CN(C)c1ccc(NC(=O)Nc2ccc(OC(F)(F)F)cc2)cc1] | 339.31234 | 4.805 | 4.397 | 53.6 |
| WF-49-BT50 | 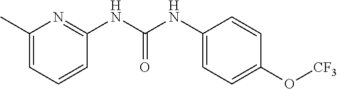 [SMILES: Cc1cccc(NC(=O)Nc2ccc(OC(F)(F)F)cc2)n1] | 311.25918 | 4.313 | 4.313 | 63.25 |
| BC-70-JK44 | 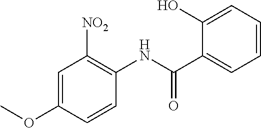 [SMILES: COc1ccc(NC(=O)c2ccccc2O)c([N+](=O)[O-])c1] | 288.25547 | 2.25 | 1.846 | 104.38 |
| ZB-20-VG22 | 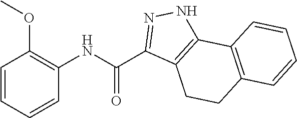 [SMILES: COc1ccccc1NC(=O)c1n[nH]c2c1CCc1ccccc1-2] | 319.35717 | 3.881 | 3.881 | 67 |
| DE-70-GX95 | 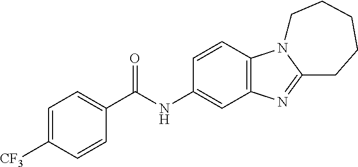 [SMILES: O=C(Nc1ccc2c(c1)nc1n2CCCCC1)c1ccc(C(F)(F)F)cc1] | 373.37162 | 4.707 | 4.826 | 46.92 |
| TC-04-QN39 | 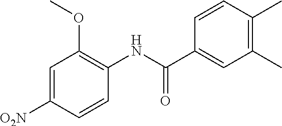 [SMILES: COc1cc([N+](=O)[O-])ccc1NC(=O)c1ccc(C)c(C)c1] | 300.30923 | 3.465 | 3.465 | 84.15 |
| BB-39-VG21 | 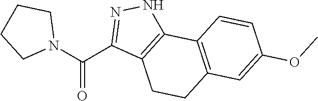 [SMILES: COc1ccc2c(c1)CCc1c(C(=O)N3CCCC3)n[nH]c1-2] | 297.35165 | 2.971 | 2.971 | 58.22 |
| PE-30-GL85 | 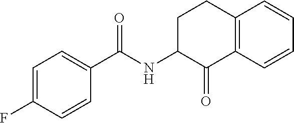 [SMILES: O=C(NC1CCc2ccccc2C1=O)c1ccc(F)cc1] | 283.29696 | 3.165 | 3.165 | 46.17 |
| DD-00-TD72 | 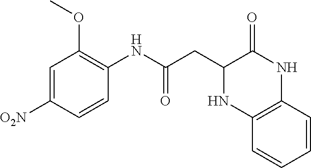 [SMILES: COc1cc([N+](=O)[O-])ccc1NC(=O)CC1Nc2ccccc2NC1=O] | 356.33274 | 1.343 | 1.36 | 125.28 |
| WC-23-HW43 | 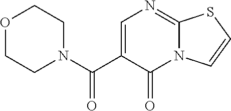 [SMILES: O=C(c1cnc2sccn2c1=O)N1CCOCC1] | 265.28834 | −1.057 | −1.057 | 87.51 |
| DA-40-RN26 | 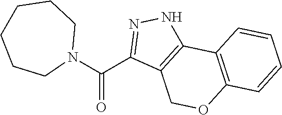 [SMILES: O=C(c1n[nH]c2c1COc1ccccc1-2)N1CCCCCC1] | 297.35166 | 3.036 | 3.036 | 58.22 |
| VC-34-CQ41 | 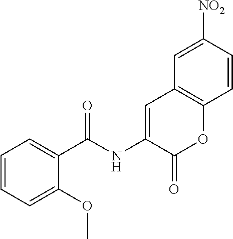 [SMILES: COc1ccccc1C(=O)Nc1cc2cc([N+](=O)[O-])ccc2oc1=O] | 340.28697 | 2.266 | 2.266 | 110.44 |
| DE-23-LP28 | 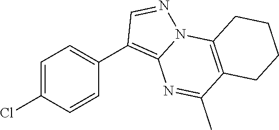 [SMILES: Cc1nc2c(-c3ccc(Cl)cc3)cnn2c2c1CCCC2] | 297.78204 | 4.088 | 4.089 | 30.19 |
| HE-23-LL28 | 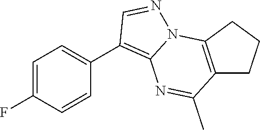 [SMILES: Cc1nc2c(-c3ccc(F)cc3)cnn2c2c1CCC2] | 267.30086 | 3.173 | 3.173 | 30.19 |
| VD-47-US03 | 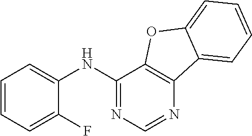 [SMILES: Fc1ccccc1Nc1ncnc2c1oc1ccccc12] | 279.2685 | 4.277 | 3.216 | 50.95 |
| RC-42-PC33 | 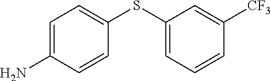 [SMILES: Nc1ccc(Sc2cccc(C(F)(F)F)c2)cc1] | 269.2854 | 4.144 | 4.143 | 51.32 |
| RA-02-XM72 | 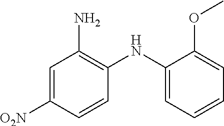 [SMILES: COc1ccccc1Nc1ccc([N+](=O)[O-])cc1N] | 259.26062 | 2.511 | 2.506 | 93.1 |
| BD-43-FC37 | 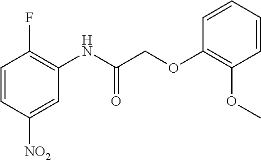 [SMILES: COc1ccccc1OCC(=O)Nc1cc([N+](=O)[O-])ccc1F] | 320.27252 | 2.533 | 2.533 | 93.38 |

TABLE 6B
Activity data of the synthesized compounds.

| | Thermal shift assay (TSA) | | | | Dual-luciferase assay | | | |
|---|---|---|---|---|---|---|---|---|
| | for HTS (Primary Screen) | | | | (Secondary Screen) | | | |
| Identifier | Rep 1 | Rep 2 | Rep 3 | Average | Rep 1 | Rep 2 | Rep 3 | Average |
| XF-84-GG52 | −54.825 | −53.167 | −47.886 | −51.95933333 | −20.127 | −9.622 | −10.689 | −13.47933333 |
| KE-95-GC86 | −44.143 | −39.907 | −46.014 | −43.35466667 | 38.732 | 22.933 | 28.926 | 30.197 |
| DE-94-ST40 | −43.197 | −38.988 | −45.804 | −42.663 | 32.017 | 4.027 | 11.984 | 16.00933333 |
| BC-46-HY47 | −49.733 | −37.65 | −40.15 | −42.511 | 11.867 | 19.333 | 22.73 | 17.97666667 |
| QE-95-GV86 | −39.252 | −46.147 | −39.605 | −41.668 | 12.075 | 21.643 | 17.009 | 16.909 |
| RB-06-II47 | −38.207 | −33.7 | −40.534 | −37.48033333 | 24.236 | 62.951 | 42.53 | 43.239 |
| NA-78-BR04 | −40.047 | −38.395 | −33.435 | −37.29233333 | 3.634 | 2.096 | 6.773 | 4.167666667 |
| NF-48-LK42 | −37.848 | −40.441 | −31.904 | −36.731 | −8.703 | −18.813 | 3.437 | −8.026333333 |
| HE-94-SP40 | −36.59 | −28.408 | −32.464 | −32.48733333 | −4.316 | 0.0875 | −3.533 | −2.587166667 |
| AE-96-GL86 | −24.426 | −32.872 | −39.682 | −32.32666667 | −10.407 | −6.737 | −3.87 | −7.004666667 |
| QE-18-WG07 | −28.711 | −35.618 | −28.938 | −31.089 | −6.883 | −1.673 | 7.962 | −0.198 |
| (QE) | | | | | | | | |
| ZB-04-ZW40 | −37.636 | −30.19 | −25.102 | −30.976 | −6.476 | −5.683 | −17.021 | −9.726666667 |
| TD-57-GF27 | −34.726 | −22.887 | −28.649 | −28.754 | −5.487 | −13.335 | −15.975 | −11.599 |
| RD-10-AY51 | −25.113 | −14.386 | −31.827 | −28.47 | 15.005 | 17.089 | 35.157 | 22.417 |
| QB-77-CJ52 | −26.177 | −27.469 | −30.242 | −27.96266667 | 73.527 | 78.157 | 57.511 | 69.73166667 |
| DC-23-NP48 | −31.736 | −22.331 | −27.257 | −27.108 | 2.683 | 4.525 | −5.12 | 0.696 |
| PB-97-IJ47 | −25.076 | −24.453 | −27.324 | −25.61766667 | 50.497 | 54.469 | 53.559 | 52.84166667 |
| VE-35-SA30 | −27.587 | −23.239 | −14.278 | −25.413 | −13.356 | −3.407 | 2.886 | −4.625666667 |
| ND-41-RW44 | −27.335 | −16.164 | −21.883 | −21.794 | −11.912 | 6.755 | −4.012 | −3.056333333 |
| WF-49-BT50 | −29.923 | −17.452 | −17.911 | −21.762 | 2.509 | 43.151 | 8.631 | 18.097 |
| BC-70-JK44 | −44.995 | −42.486 | −42.021 | −43.16733333 | −15.755 | 19.661 | −7.706 | −1.266666667 |
| ZB-20-VG22 | −40.648 | −34.979 | −25.251 | −33.626 | 78.658 | 54.271 | 28.098 | 53.67566667 |
| DE-70-GX95 | −32.487 | −32.225 | −32.154 | −32.28866667 | 10.293 | 19.466 | 4.859 | 11.53933333 |
| TC-04-QN39 | −29.973 | −32.212 | −31.408 | −31.19766667 | −0.223 | −3.717 | 5.708 | 0.589333333 |
| BB-39-VG21 | −25.313 | −30.855 | −24.351 | −26.83966667 | 7.916 | 6.944 | −16.55 | −0.563 |
| PE-30-GL85 | −25.324 | −17.383 | −27.633 | −26.4785 | 23.457 | 28.61 | 39.387 | 30.48466667 |
| DD-00-TD72 | −26.061 | −27.529 | −24.462 | −26.01733333 | −6.846 | −4.156 | −23.8 | −11.60133333 |
| WC-23-HW43 | −23.891 | −27.623 | −2.873 | −25.757 | 3.08 | −4.363 | −4.438 | −1.907 |
| DA-40-RN26 | −32.624 | −23.105 | −16.697 | −24.142 | 8.675 | 30.307 | 18.292 | 19.09133333 |
| VC-34-CQ41 | −20.551 | −23.48 | −16.337 | −20.12266667 | −1.148 | 0.633 | −1.478 | −0.664333333 |
| DE-23-LP28 | −41.891 | −47.531 | −42.357 | −43.92633333 | −1.44 | −2.935 | 8.312 | 1.312333333 |
| HE-23-LL28 | −33.525 | −34.104 | −31.216 | −32.94833333 | −3.326 | 0.781 | 11.755 | 3.07 |
| VD-47-US03 | −30.89 | −33.868 | −21.305 | −32.379 | 62.195 | 68.951 | 58.525 | 63.22366667 |
| RC-42-PC33 | −27.008 | −29.656 | −29.739 | −28.801 | 0.302 | −4.289 | −8.439 | −4.142 |
| RA-02-XM72 | −28.732 | −19.148 | −9.793 | −23.94 | −6.355 | −4.094 | −7.431 | −5.96 |
| BD-43-FC37 | −6.487 | −21.052 | −22.537 | −21.7945 | 30.921 | 44.067 | 26.686 | 33.89133333 |

### Example 10

A head-to-head comparison between the newly discovered SALL4 degrader QE-18-WG07 and IMiDs (Pomalidomide and Thalidomide) was performed. To assess the differential isoform degradation and biological consequences induced by QE-18-WG07 and IMiDs in cancer cells, the inventors employed the newly developed SALL4 isoform-specific cellular luciferase reporter system in Example 1 (FIG. 1A), and human HCC cell lines (SNU-398 and SNU-182), and NSCLC cell line H661 that have a high SALL4 level endogenously. The inventors treated H1299 cells overexpressing SALL4B_Nluc and the HCC (SNU-398, SNU-182) and NSCLC cells (H661) with increasing concentrations of QE-18-WG07 and IMiDs, and assayed the SALL4B_Nluc, as well as the endogenous SALL4A and B levels by immunoblots described in Example 4.

### Results and Discussion

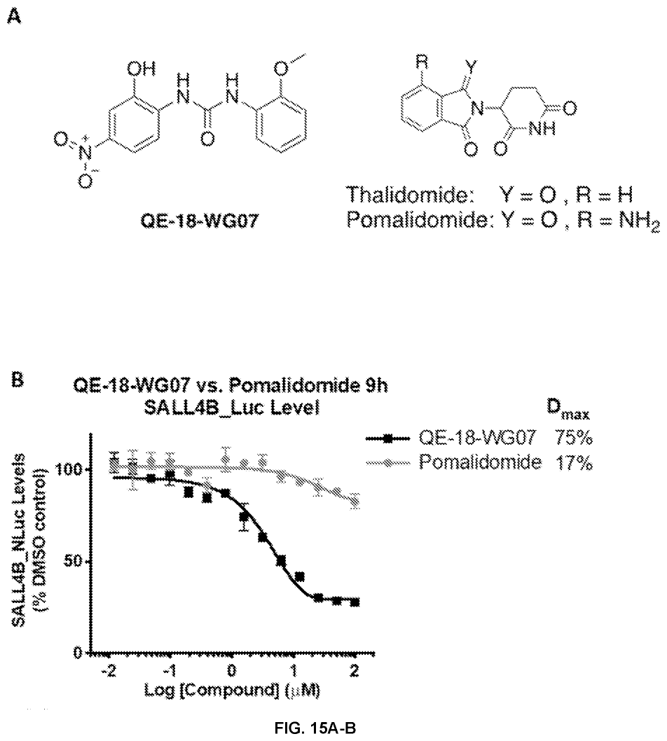

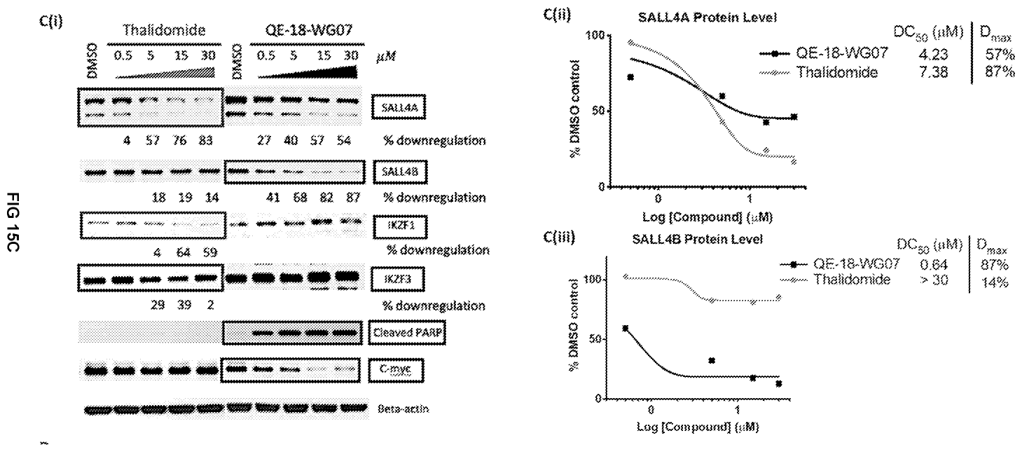

When treated with QE-18-WG07, a pronounced loss of SALL4B protein was observed in both the SALL4B dual-luciferase reporter assay with maximum degradation (Dₘₐₓ)=75% after 9 hours of treatment (FIG. 15B) and SNU-398 HCC cells with Dₘₐₓ=87% after 24 h of treatment (FIG. 15C). IMiDs (Pomalidomide and Thalidomide), on the other hand, did not induce noticeable change in the level of SALL4B_Nluc in the luciferase reporter assay (Dₘₐₓ=17%, FIG. 15B). Consistently, in HCC cells, IMiDs also had a limited effect on the endogenous SALL4B level with Dₘₐₓ=19% (FIG. 15C). Further studies revealed dose-proportional degradation of endogenous SALL4B upon QE-18-WG07 treatment in a panel of SALL4-mediated cancers, including SNU398, SNU-182 HCC, and H661 lung cancer cells (FIG. 15D). These results demonstrate that degradation of the oncogenic SALL4B isoform in SALL4-mediated cancer cells can only be achieved with QE-18-WG07 treatment, not IMiDs.

The ability of QE-18-WG07 in degrading the endogenous SALL4 isoforms in HCC cells was compared with that of IMiDs. QE-18-WG07 potently degraded SALL4B with a DC₅₀ of ˜0.64 μM and Dₘₐₓ=87% but was far less effective at degrading SALL4A (Dₘₐₓ=57%, DC₅₀˜4.23 μM). In contrast, IMiDs efficiently degraded endogenous SALL4A (Dₘₐₓ=83%, DC₅₀=7.38 μM), but not SALL4B isoform (Dₘₐₓ=19%, DC₅₀>30 μM) in SNU-398 cells (FIG. 15C).

The inventors further evaluated the selectivity of QE-18-WG07 by monitoring the degradation of IKZF1 and IKZF3, which are previously described neo-substrates of CRBN upon IMiDs treatment (Lu, G. et al. Science 2014, 343, 305-309; and Kronke, J. et al. Science 2014, 343, 301-305), with immunoblots in SNU-398 HCC cells. Compared to Thalidomide, which still effectively depleted IKZF1 (Dₘₐₓ=64%) and IKZF3 (Dₘₐₓ=39%) in SNU-398 cells, QE-18-WG07 did not degrade IKZF1 and 3 (FIG. 15C). These results imply that QE-18-WG07 is selective for protein targets that are different from IMiDs for degradation, and hence, could elicit differential biology and phenotype in cancer cells.

Indeed, 24 h treatment of QE-18-WG07 in SNU-398 HCC cells led to efficient suppression of SALL4 downstream target, c-myc, in a dose-dependent manner (IC₅₀˜5.12 μM). It also led to substantial apoptosis induction, as evident from the prominent cleavage of PARP in the immunoblot (FIG. 15C). Treatment with Thalidomide, however, did not affect the level of c-myc and there was no cleavage of PARP observed, implying a lack of apoptotic advantage for IMiDs treatment in SNU-398 HCC cells (FIG. 15C). Indeed, while QE-18-WG07 induced a potent and superior inhibitory effect on SNU-398 HCC cell proliferation after 72 h with an IC₅₀ of 280 nM, both of the IMiDs, Pomalidomide and Thalidomide, did not result in any antiproliferative consequences (FIG. 15E).

Together, these data exemplify that differential isoform degradation by IMiDs and QE-18-WG07 lead to differential biological consequences in cancer cells. As compared to IMiDs, QE-18-WG07 targets SALL4B isoform for degradation more efficiently, which was demonstrated earlier as the critical isoform in conferring a survival advantage to SALL4-mediated cancer cell, hence leading to SALL4-mediated cancer cell death and demonstrating anti-cancer advantage over IMiDs. Furthermore, by selectively targeting SALL4B isoform while leaving the majority of SALL4A isoform intact, QE-18-WG07 represents a safer option for cancer therapeutics than IMiDs which was known for their teratogenicity effect due to SALL4A isoform degradation.

### Example 11

Having established the feasibility of achieving SALL4B isoform degradation by QE-18-WG07 in HCC cells, the inventors next endeavoured to gain deeper insights into its mechanism of action. The inventors first investigated the stoichiometry of SALL4 engagement by QE-18-WG07 via saturating the SALL4 (1-300aa) protein with QE-18-WG07 and evaluating the shift in melting temperature (ΔTₘ) of SALL4 (1-300aa).

### Stoichiometry of SALL4 Engagement by QE-18-WG07

To experimentally assess the stoichiometry of the binding between SALL4 (1-300aa) and QE-18-WG07, the inventors titrated 13.8 μM of purified recombinant SALL4 (1-300aa) protein with 12 increasing concentrations of QE-18-WG07 and evaluated the dose-response correlation in the ΔTₘ shift by following the protocol for TSA in Example 7.

### Results and Discussion

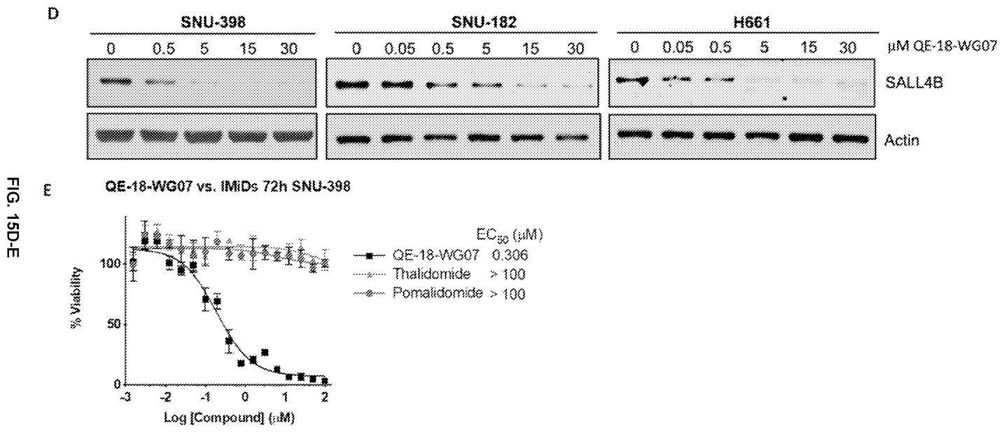

After 30 min of incubation, the inventors observed that there was a positive shift in the melting temperature (Tₘ) of SALL4 (1-300aa) in the presence of an increasing concentration of QE-18-WG07, implying that QE-18-WG07 bound and stabilized SALL4 (1-300aa). More importantly, ΔTₘ of SALL4 (1-300aa) increased with low concentrations of QE-18-WG07 and plateaued at higher concentrations. This trend is consistent with saturation of SALL4 (1-300aa) binding sites by excess ligand at a high concentration of QE-18-WG07. Notably, the saturation was observed to occur at 12.5 μM of QE-18-WG07 or higher, suggesting that the binding stoichiometry between SALL4 and QE-18-WG07 is 1:1; as 12.5 μM of QE-18-WG07 to 13.8 μM of SALL4 (1-300aa) is approximately at a ratio of 1:1. (FIG. 16A and Table 7) Therefore, by using TSA to monitor the binding of QE-18-WG07 to the first 1 to 300 amino acids of SALL4, the inventors have demonstrated that the compound engages to this specific 1-300 N-terminal domain of SALL4 via a stoichiometric 1:1 binding.

TABLE 7
Temperature shifts of SALL4(1-300) protein versus indicated
concentrations of QE-18-WG07 after 30 min of incubation.

| | | |
|---|---|---|
| QE-18-WG07 | SALL4 (1-300) Melting | |
| Concentration | Temperature Shift (° C.) | Standard |

| (μM) | Rep 1 | Rep 2 | Deviation |
|---|---|---|---|
| 50.0000 | 1.273 | 0.983 | 0.205 |
| 25.0000 | 1.273 | 1.273 | 0.000 |
| 12.5000 | 1.373 | 1.083 | 0.205 |
| 6.2500 | 0.703 | 0.703 | 0.000 |
| 3.1250 | 0.413 | 0.793 | 0.269 |
| 1.5625 | 0.503 | 0.503 | 0.000 |
| 0.7813 | −0.167 | −0.167 | 0.000 |
| 0.1953 | −0.267 | −0.267 | 0.000 |
| 0.0977 | −0.357 | −0.357 | 0.000 |
| 0.0488 | −0.077 | −0.357 | 0.198 |
| 0.0122 | −0.267 | −0.267 | 0.000 |
| 0.0061 | −0.357 | −0.357 | 0.000 |

### Example 12

The inventors then explored the SALL4 degradation pathway mediated by QE-18-WG07, using SNU-398, SNU-182, and H661 cancer cell lines. The inventors hypothesized that the degradation of SALL4 by the lead degrader could be mediated via the ubiquitin-proteasome system (UPS). To test this hypothesis, the inventors performed rescue experiments where the UPS pathway is either blocked by a proteasome inhibitor, MG132 or a Nedd8 activating enzyme inhibitor, MLN4924, which is important for E3 complex activation. Furthermore, the effect of the degradation of SALL4 by QE-18-WG07 on the degradation of SALL4 protein binding partner (retinoblastoma binding protein 4 (RBBP4)) was evaluated by fluorescence polarization

### Rescue Experiments for the Blocking of the UPS Pathway

26S proteasome complex and neddylation was inhibited by cotreating MG132 at 1 and 3 μM and MLN4924 at 10 and 15 μM for 18 h, 20 h, 24 h or 48 h with desired concentrations of QE-18-WG07 in SNU-398, H661 and SNU-182 cells. After 18 h or 24 h of treatment, cells were then lysed and subjected to further protein extraction and immunoblotting by following the protocol in Example 4. For caspase glo assay, 20 h of cotreatment was performed by following the protocol in Example 5. For viability assay, 48 h of cotreatment was carried out by following the protocol in Example 5.

### Results and Discussion

The inventors found that co-treatment of QE-18-WG07 in SNU-398 cells with either MG132, or MLN4924 attenuated the SALL4 degradation induced by QE-18-WG07 (FIG. 16B). This suggests that the degradation of SALL4 by QE-18-WG07 requires both proteasome function and E3 complex activation by the Nedd8 enzyme. Collectively, these data demonstrate that QE-18-WG07 treatment promotes SALL4 degradation in cells via an E3 ligase-mediated and proteasome-dependent mechanism. As expected, when QE-18-WG07 can no longer degrade SALL4 in the presence of MG132 and MLN4924, the expression of SALL4 downstream target c-myc (Moeini, A. et al., J. Hepatol. 2017, 66, 952-961), was found to be rescued (FIG. 16B). Previously, the inventors reported that in cancer cells, SALL4 associates with nucleosome remodeling deacetylase (NURD) to silence tumor-suppressor genes (Gao, C. et al., Blood 2013, 121, 1413-1421; and Liu, B. H. et al. Proc. Natl. Acad. Sci. U.S.A. 2018, 115, E7119-E7128).

RBBP4 is a subunit of NuRD, which acts as a chaperone in nucleosome assembly (Zhang, W. et al., Nat. Struct. Mol. Biol. 2013, 20, 29-35). Therefore, the inventors further examined if the degradation of SALL4 by QE-18-WG07 could also result in the degradation of SALL4 protein binding partner, RBBP4. Upon treatment with QE-18-WG07 in SNU-398 cells, there was no observable effect on the level of RBBP4, indicating no off-target activity of QE-18-WG07 towards NuRD, the protein that SALL4 forms complex with (FIG. 16B). Similarly, in SNU-182 and H661 cells, SALL4 degradation by QE-18-WG07 was hindered by proteasome inhibitor MG132 and by MLN4924, which inhibits E3 complex activation (FIG. 16C).

Therefore, QE-18-WG07 engages and efficiently induces robust degradation of SALL4, especially SALL4B, in both HCC and lung cancer cell lines via the ubiquitin-proteasome pathway.

### Example 13

To investigate which specific E3 ligase is responsible for the targeted SALL4 degradation, the inventors systematically performed Cellular Thermal Shift Assay (CETSA) to discover the specific E3 ligase engaged by QE-18-WG07, and subsequently carried out genetic knockdown of the identified E3 ligase to confirm that it is essential for QE-18-WG07-induced SALL4 degradation in SNU-398 cells.

### Cellular Thermal Shift Assay

Cells were washed with cold PBS and harvested into 50 mL tubes and centrifuged at 1,200×g for 5 min. For cell lysis, cells were resuspended in 1× NP-40 lysis buffer containing 10 mM Tris-HCl (pH 7.4), 150 mM NaCl, 2.7 mM KCl and 0.4% NP-40, and rotated end-over-end at 4° C. for 30 min. The cell lysate was centrifuged at 20,000 rpm for 10 min at 4° C. and the supernatant was collected. The cell lysate was divided into two aliquots, with one aliquot being treated with QE-18-WG07, and the other aliquot with DMSO control. After specific incubation time at room temperature, the respective lysates were divided into smaller aliquots of 40 μL and heated individually at different temperatures (51-56° C.) for 3 min in a thermal cycler, followed by a 3-min cool-down at room temperature. The heated lysates were centrifuged at 15,000 rpm for 10 min at 4° C. to separate the soluble fractions from the precipitates. The supernatants were transferred to new microtubes and analyzed by SDS-PAGE, followed by western blot analysis described in Example 4. The integrated band intensities were quantified by ImageJ. The thermal curves were analyzed by nonlinear regression using Graph Pad Prism.

### Rescue Experiment by Lentiviral Cereblon (CRBN) Knockdown

shRNA targeting CRBN (sh-CRBN) or a scrambled control (sh-scr) with the following sequences were cloned into the pLKO. 1 vector:

sh-scr:
5′-CAACAAGATGAAGAGCACCAA-3′
sh-CRBN:
5′-CAGGATAGTAAAGAAGCCAAA-3′

For lentiviral transduction, SNU-398 cells were spun with lentiviral particles containing the aforementioned vectors in RPMI (plus 10% FBS, 8 μg/mL polybrene) at 800 g for 60 minutes at room temperature. 96 h post-infection, cells were seeded in 6-well or 384-well plates and incubated overnight at 37° C., 5% CO₂. Cells were then treated with DMSO or QE-18-WG07 at desired concentrations (0.5% final DMSO) for 18 h and 24 h. Immunoblot analysis was subsequently performed with the indicated antibodies by following the protocol in Example 4. For cell titer glo viability assay (described in Example 5), cells were treated for 72 h at 37° C., 5% CO₂.

### Results and Discussion

The CETSA methodology is based on sequential thermal denaturation and irreversible aggregation of target protein, a process that can be altered by the presence of a ligand. The separation of the remaining soluble protein and irreversible aggregates is achieved either prior to detection through centrifugation or filtration. CETSA revealed target engagement of QE-18-WG07 to the E3 ligase, CRBN. Thermal profiling of CRBN in SNU-398 cell lysates incubated with QE-18-WG07 (50 μM) yielded a sizable stabilization with an overall thermal shift of up to 5° C., from 51 to 56° C. (FIG. 16D). The dose dependency of CRBN thermal stabilitization by QE-18-WG07 was confirmed at 53.4° C. as interpreted from the isothermal dose-response CETSA fingerprints (FIG. 16E). This data suggests that QE-18-WG07 is a CRBN glue molecule and modulator. To get a definite confirmation of the cellular requirement for CRBN in the degradation action of QE-18-WG07, CRBN was knocked down (KD) in the SNU-398 HCC cell line. Treatment of QE-18-WG07 in SNU-398 cells with scrambled shRNA promoted degradation of SALL4 in a dose-dependent manner, whereas exposure of CRBN-KD SNU-398 cells to increasing concentrations of QE-18-WG07 was ineffectual where the level of SALL4 protein was unperturbed (FIG. 16F). Therefore, down-regulation of CRBN by short hairpin RNA (shRNA) in SNU-398 cells prevented the degradation of endogenous SALL4B by QE-18-WG07 (FIG. 16F). These data provide mechanistic evidence for CRBN-dependent proteasomal degradation of SALL4 by QE-18-WG07, and that this compound is a CRBN glue molecule.

### Example 14

To address whether the CRBN-dependent proteasomal degradation of SALL4 was responsible for the anti-proliferative and cytotoxic effects of QE-18-WG07 in SALL4-mediated cancer cells, the inventors evaluated the drug sensitivity to QE-18-WG07 of a panel of cancer cell lines with differential dependency on SALL4 for survival. In addition, the inventors also monitored the changes in apoptotic response and anti-viability effect (described in Example 5) induced by drug treatment upon the chemical and genetic rescue of drug-mediated SALL4 degradation by following the protocols in Examples 12 (chemical rescue) and 13 (genetic rescue).

### Rescue Experiment by Lentiviral SALL4A or B Isoform Overexpression

Lentiviral constructs for SALL4A and SALL4B cloned in pFUW were used for overexpression. For lentiviral transduction, SNU-398 cells were spun with viral particles containing empty pFUW vector, SALL4A and SALL4B in RPMI (10% FBS, 5 μg/mL polybrene) at 800 g for 60 min at 32° C. Transduction efficiency was assessed via mCherry signal using BD FACSAria™. Following 3 days past infection, the cells were selected using puromycin (1 μg/mL) for one week. The overexpression was verified using qRT-PCR and western blot (described in Example 4). qRT-PCR was carried out as described in the analytical techniques section with qRT-PCR System (Thermo Fisher Scientific) according to the manufacturer's instructions. The cells were subsequently seeded for analysing drug sensitivity.

### Results and Discussion

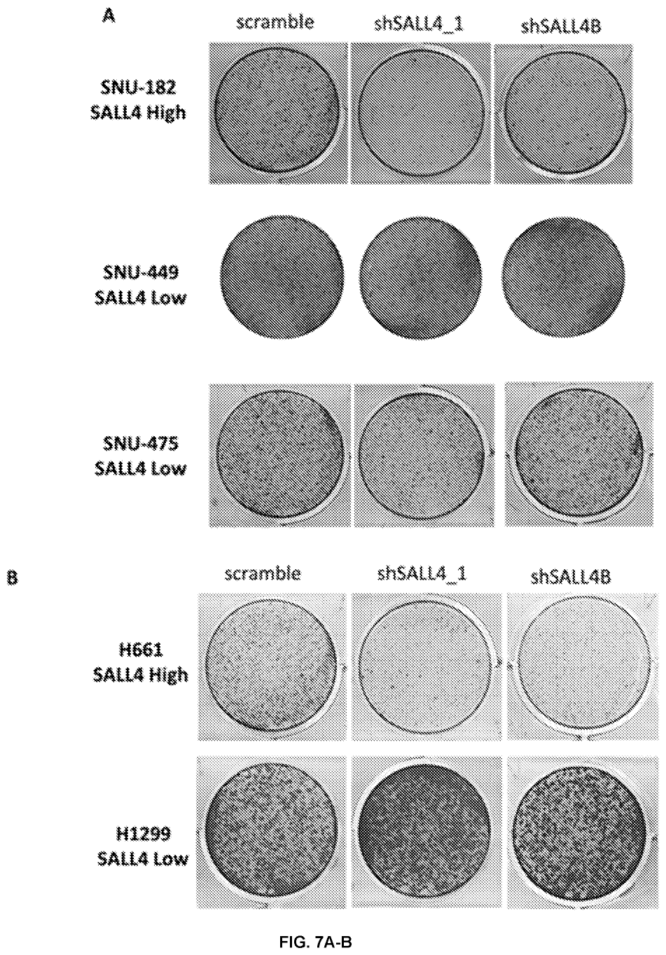

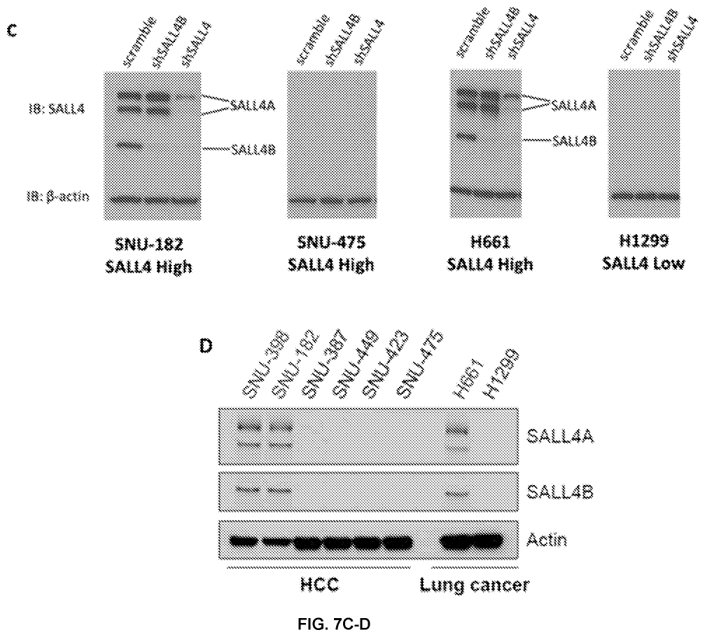

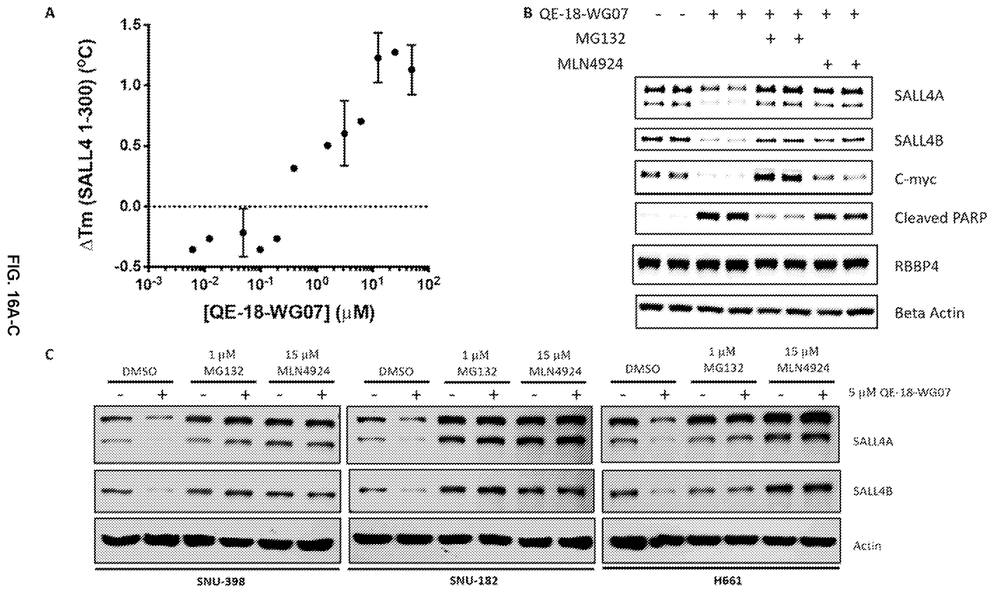

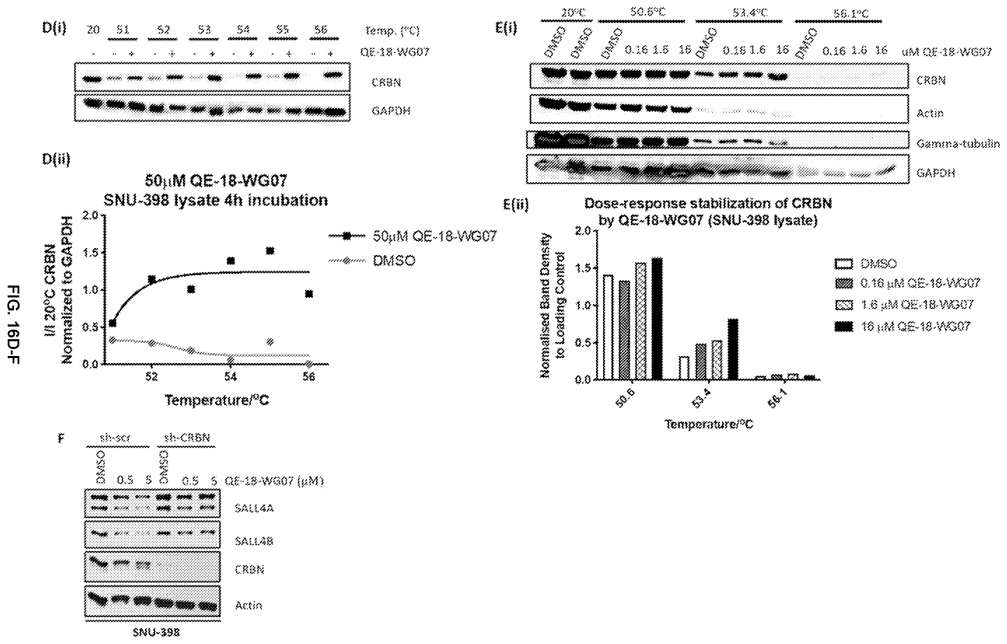

To this end, a panel of five HCC cell lines and a pair of lung cancer cell lines with differential expression levels of SALL4 (FIG. 7D) and differential dependency on SALL4 for survival were evaluated for their drug sensitivity to QE-18-WG07 treatment. Previously, the inventors established the dependency of the SALL4-high, not SALL4-low cells in this panel on SALL4, in particular SALL4B, for survival via shRNA knockdown in these cells. (FIGS. 6 and 7) All three SALL4-high and SALL4B-dependent SNU-398, SNU-182 and H661 cancer cells showed marked sensitivity to QE-18-WG07, with maximum growth inhibition Eₘₐₓ=90-100% achieved at [QE-18-WG07]=1 μM and potent nanomolar IC₅₀ of 397 nM, 951 nM and 522 nM, respectively (FIG. 17A). In contrast, SNU-387, SNU-449, SNU-475, and H1299 cells, which have low SALL4 expression and are not dependent on SALL4B for survival, were relatively less sensitive to QE-18-WG07, where approximately 30-60% of cells remained viable despite being exposed to the highest dose of the compound at 30 μM (FIG. 17A). The data suggest that QE-18-WG07 is potent and selective against SALL4-high expressing HCC and NSCLC cell lines, and hence will be potentially effective in treating tumors driven by SALL4 with minimal off-target toxicity.

Notably, in all the three SALL4-high sensitive cell lines with observed inhibition of proliferation following QE-18-WG07 treatment, the inventors have previously observed that SALL4B was degraded by QE-18-WG07 in a dose-dependent manner (FIG. 15D). Degradation of SALL4 by QE-18-WG07 subsequently led to induction of the apoptotic response in these cells, as evidenced by prominent activation of caspase (FIG. 17B). Kinetic studies revealed that the enhanced apoptotic marker cleaved PARP in SNU-398 cells was observed after SALL4 degradation by QE-18-WG07 (FIG. 17C).

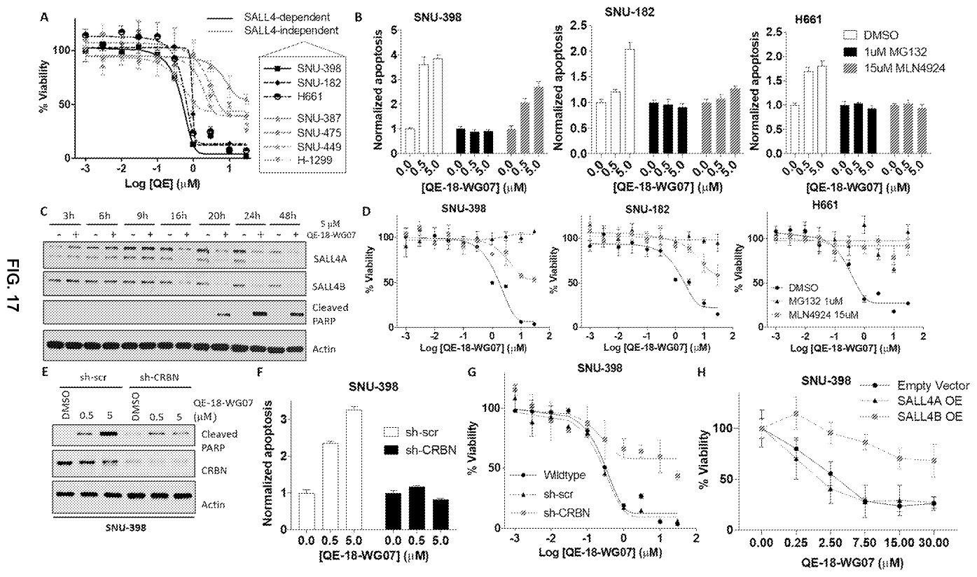

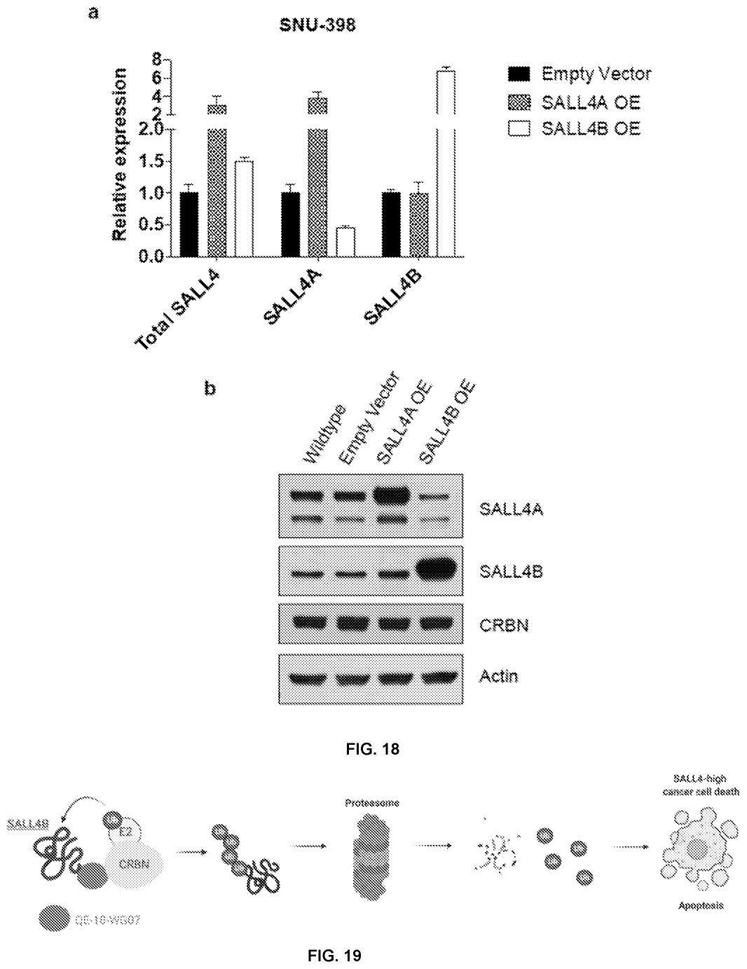

Notably, as SALL4B degradation by QE-18-WG07 was chemically rescued by MG132 or MLN4924 cotreatment, the observed increased apoptotic response and cancer cell growth inhibition triggered by drug treatment in SNU-398, SNU-182, and H661 cancer cells were attenuated (FIG. 17B and D). Furthermore, when SALL4B degradation by QE-18-WG07 was hindered by CRBN knockdown in SNU-398 cells, induction of the apoptotic marker cleaved PARP and caspase activation upon drug treatment were also abrogated (FIG. 17E-F). Indeed, ablation of cereblon-dependent SALL4B degradation conferred QE-18-WG07 resistance to CRBN-KD SNU-398 cells (FIG. 17G). In addition, QE-18-WG07 showed a marked reduction in cytotoxicity against SNU-398 cells with overexpressed SALL4B, while empty vector control cells and SALL4A-overexpressed SNU-398 cells exhibited similar dose-proportionate sensitivity to QE-18-WG07 treatment (FIG. 17H and 18). Collectively, these findings strongly corroborate that the cytotoxicity effect of QE-18-WG07 against SALL4-mediated cancer largely depends on its SALL4B degradation activity.

Hence, combining the results obtained here and in Examples 11-14, the following mechanism of action for how QE-18-WG07 effectively targets SALL4B for degradation and induce anti-cancer effects in SNU-398 HCC cell was proposed: the compound first recruits SALL4B by binding to the protein's first 1-300 N-terminal domain and the E3 ligase CRBN to close proximity, where ubiquitin molecules from the E2-E3 ligase complex will be transferred to the SALL4B protein. The poly-ubiquitinylated SALL4B will be targeted for degradation by the cellular proteasome, resulting in SALL4B depletion and subsequent cell apoptosis and cell death of SALL4-mediated cancer cells (FIG. 19).

### Example 15

The robust targeted SALL4 degradation activity and the resulting selective anti-proliferative effects against SALL4-driven cancer cells of QE-18-WG07 in vitro established the feasibility of modelling the therapeutic opportunity of this compound in vivo in a SALL4-high HCC xenograft model of human SNU-398 HCC cells.

### HCC Mouse Xenograft

Animals were maintained, and all animal work were performed following the protocols approved by the Institutional Animal Care and Use Committee. The SNU-398 cell line was cultured as described above in ‘Cell Culture’. To establish the SNU-398 xenografts, NOD.Cg−Prkdc^{sc}^{ⁱ}^{d}Il2rg^{tm}^{¹}^{Wjl}/Sz (NSG) female mice (6-8 weeks) were anesthetized using 2% isoflurane USP, and 0.5×10⁶ SNU-398 cells in 200 μL of 1:1 RPMI:Matrigel were subcutaneously injected to the mouse flanks. When tumors were palpable, mice were randomized to receive either QE treatment or vehicle containing 5% DMSO, 12.5% Ethanol, and 12.5% Cremophor EL in 70% 1× PBS. Freshly prepared vehicle or QE-18-WG07 was administered by intraperitoneal (IP) injection, once daily (qd) at 10 mg/kg in the pilot study and 5, 15, and 45 mg/kg for dose-expansion studies. Before each drug injection, mouse weight and tumor volume were monitored. Mice were euthanized once tumors reached more than 1.5 cm in diameter. The tumors were harvested, imaged and snap-frozen for storage at −80° C. until later use.

### Results and Discussion

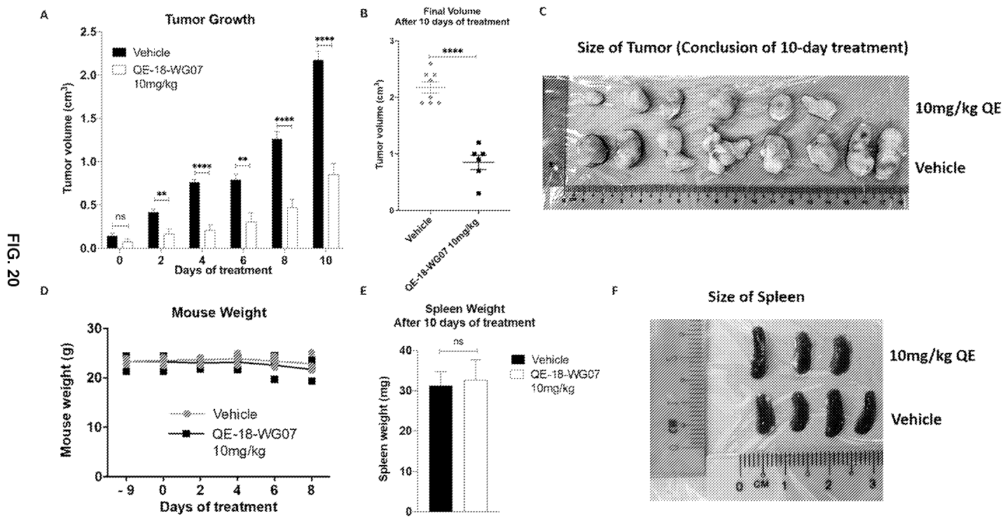

In the pilot experiment, the inventors first evaluated the tolerability and antitumor efficacy of QE-18-WG07 at a single dose of 10 mg/kg in a SALL4-high HCC xenograft model of human SNU-398 HCC cells. Tumor-bearing mice were administered with QE-18-WG07 or vehicle control by IP injection (10 mg/kg body weight daily). After 10 days of therapy, a first tumor in the vehicle control group reached institutional limits for tumor size, and the study was terminated for comparative assessment of efficacy. Administration of QE-18-WG07 at 10 mg/kg daily was observed to markedly attenuate SNU-398 tumor progression, as determined by serial volumetric assessment (FIG. 20A). When the tumor weight was assessed post-mortem after 10 days of therapy, it was observed that QE-18-WG07 markedly inhibited tumor growth and reduced tumor sizes by 64% in the treated mice as compared to the vehicle control group (FIG. 20B-C). Notably, 10 days of daily QE-18-WG07 treatment at 10 mg/kg was well tolerated by the mice, as interpreted from the animal weight preservation (FIG. 20D) and normal spleen weight (FIG. 20E-F).

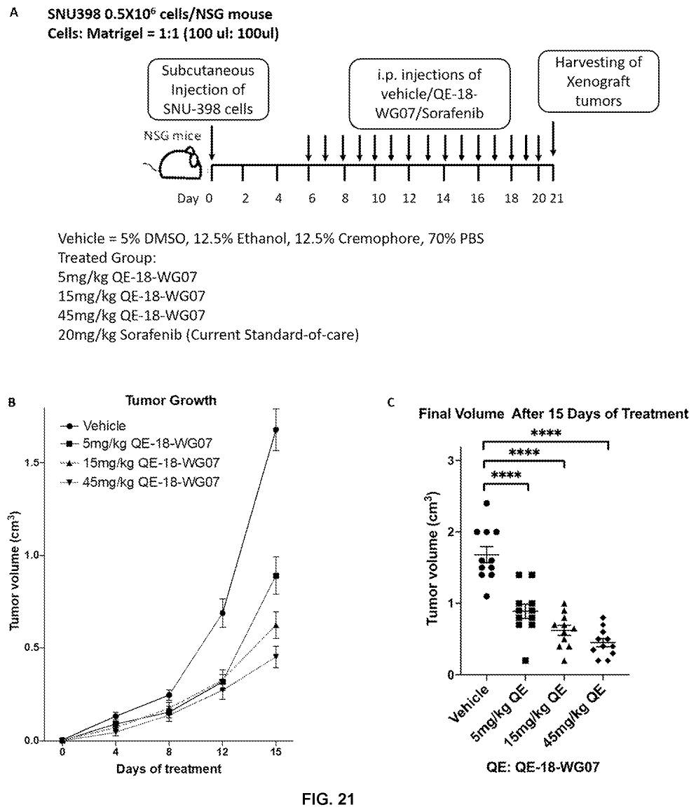

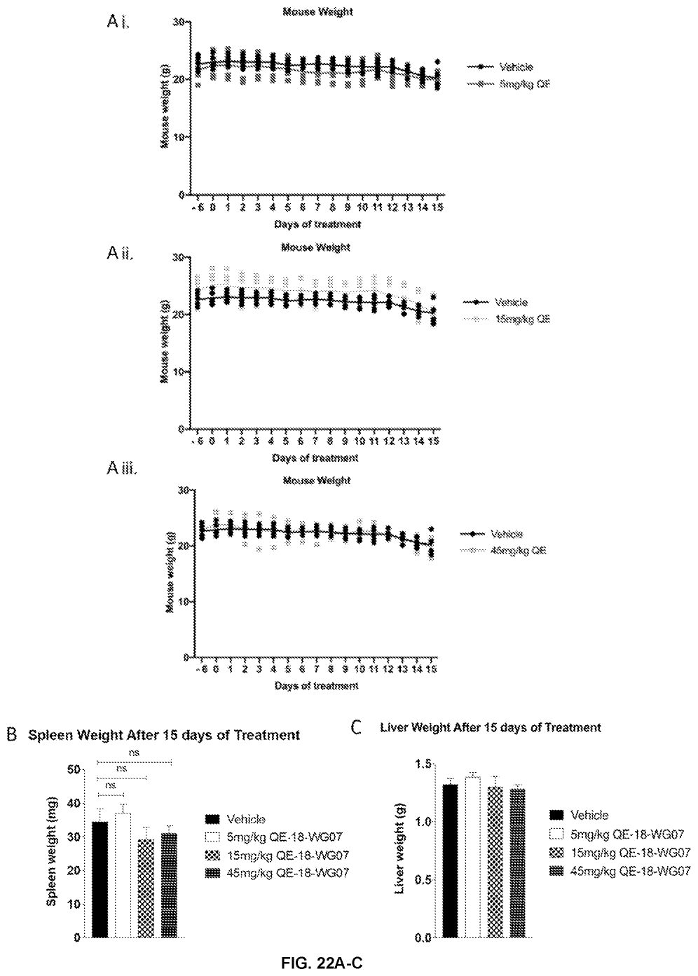

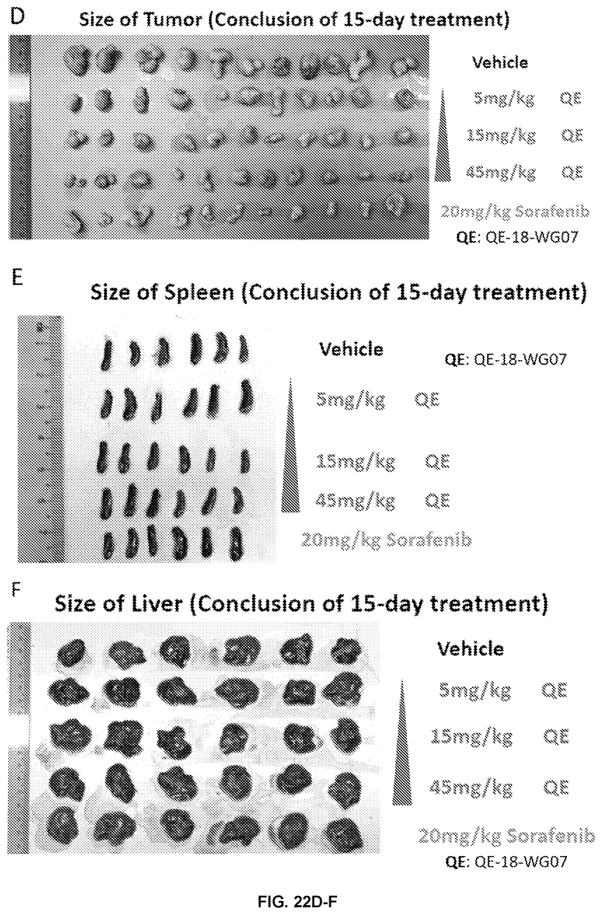

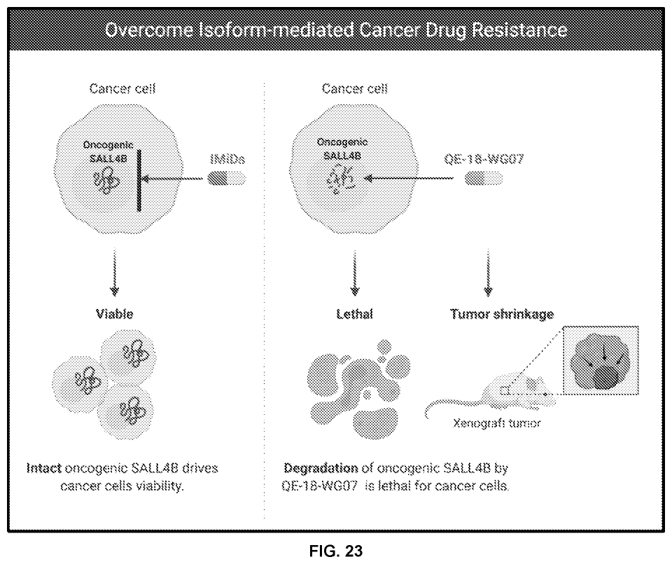

Next, the inventors employed the same xenograft model to conduct a larger-scale core study to further investigate the therapeutic window of QE-18-WG07 by assessing its toxicity and anti-tumor effects at three different doses. Once the SNU-398 tumors were established, the mice were randomized to receive 5, 15, or 45 mg/kg of QE-18-WG07 or vehicle control by IP injection daily for two weeks (FIG. 21A). The dose-expansion study showed that effective suppression of tumor growth in all the mice was achieved with daily administration at all three doses of QE-18-WG07 (FIG. 21B). Post-mortem analysis of tumor weight also revealed significant tumor regression with increasing QE-18-WG07 administration. After 15 days of treatment at 5 mg/kg, QE-18-WG07 induced a tumor shrinkage by 40%, which further decreased by 60% and 70% at 15 mg/kg and 45 mg/kg of QE-18-WG07, respectively (FIG. 21C and 22D). Hence, pharmacologic destabilization of SALL4B by QE-18-WG07 exhibited in vivo anti-tumor advantage in a human SALL4-mediated HCC xenograft. Notably, daily treatment at the highest dose of 45 mg/kg for 2 weeks was still well tolerated by the mice, as evidenced by negligible weight loss (FIG. 22A) and normal spleen (FIG. 22B and E) and liver weight (FIG. 22C and F).

In summary, the inventors demonstrated the in vivo anti-tumor advantage and therapeutic window of the newly identified CRBN modulator and SALL4 degrader QE-18-WG07. The in vitro SALL4B knockdown and in vivo SALL4B gain-of-function study testified that SALL4B is the dominant oncogenic isoform in cancer and should be the key for therapeutic intervention. The findings highlight the superiority of QE-18-WG07 over IMiDs as a potent anti-tumor agent against SALL4-mediated cancers and demonstrate the potential of QE-18-WG07 for the treatment of SALL4-mediated cancers.

1. Use of a compound of formula I: 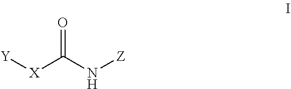
where:
X represents a bond or NH;
Y represents: 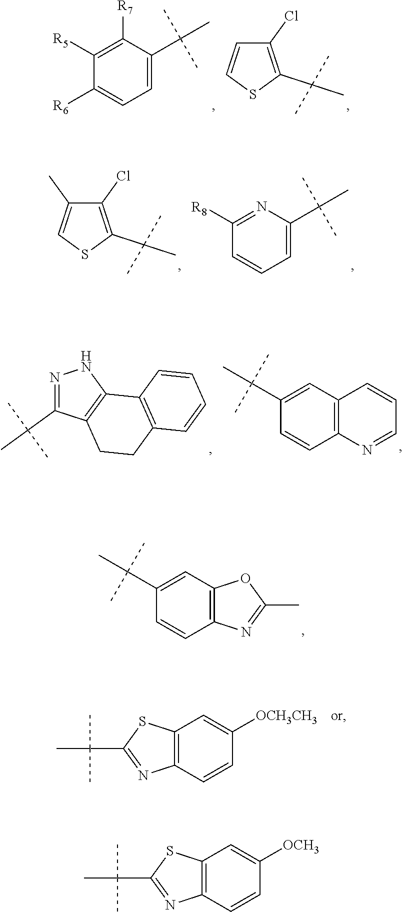
where the dotted lines represent the point of attachment to the rest of the molecule;
Z represents: 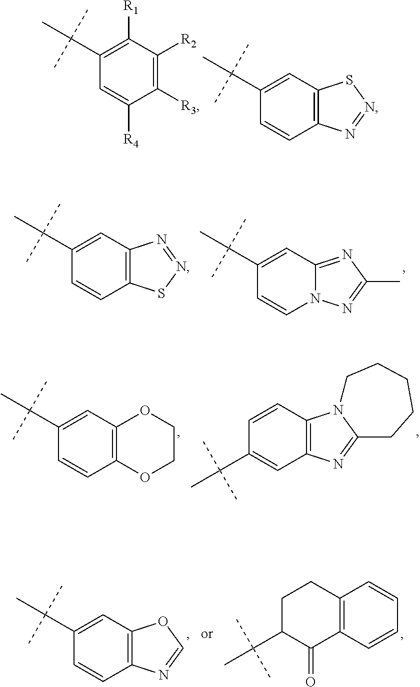
R¹ represents H, Cl, F, OCF₃, OCH₃ or NO₂;
R² represents H, Cl, or OCH₃;
R³ represents H, F, CF₃, OCF₃, CH₃, OCH₃; CH₂CH₃, Cl, —C(═O)OCH₃ or NO₂;
R⁴ represents H, Cl or CH₃;
R⁵ represents H, Cl or F;
R⁶ represents H, CF₃, NO₂, N (CH₃)₂, or 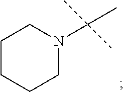
R⁷ represents H or OH;
R⁸ represents H or CH₃,
or a pharmaceutically acceptable salt, solvate or derivative thereof, in the preparation of a medicament to treat cancer,
provided that:
the compound is not 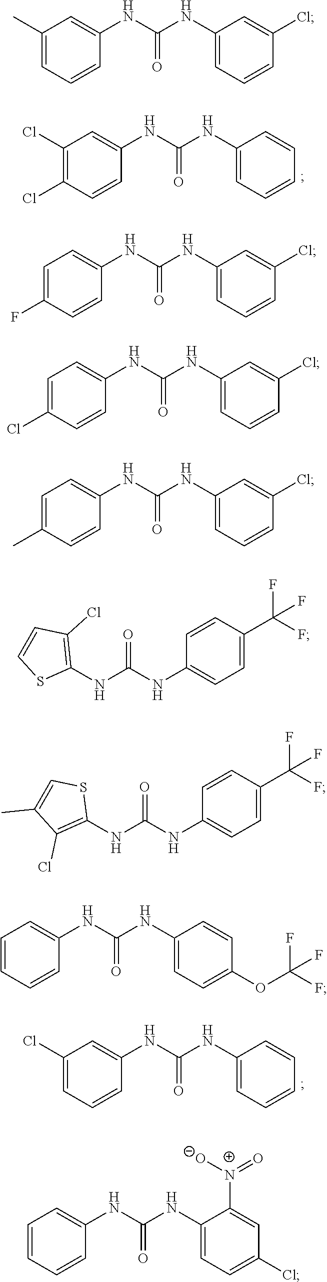 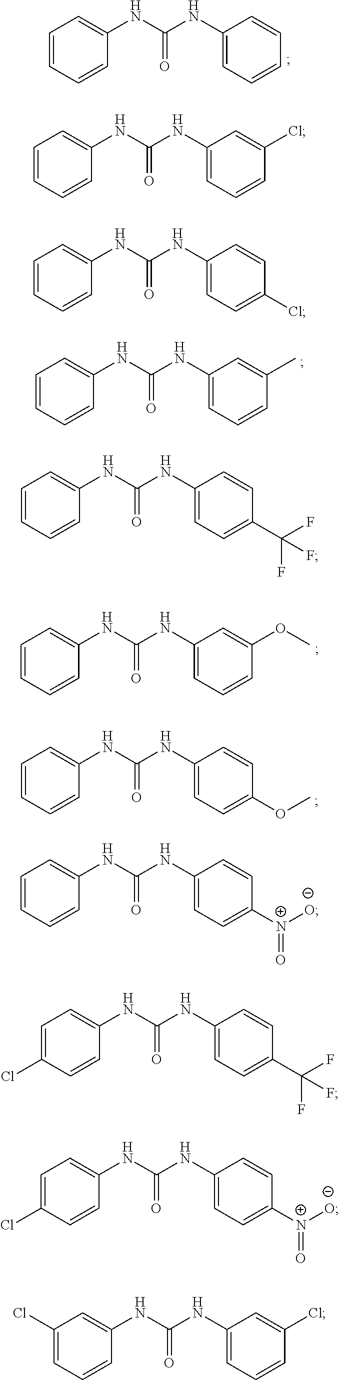 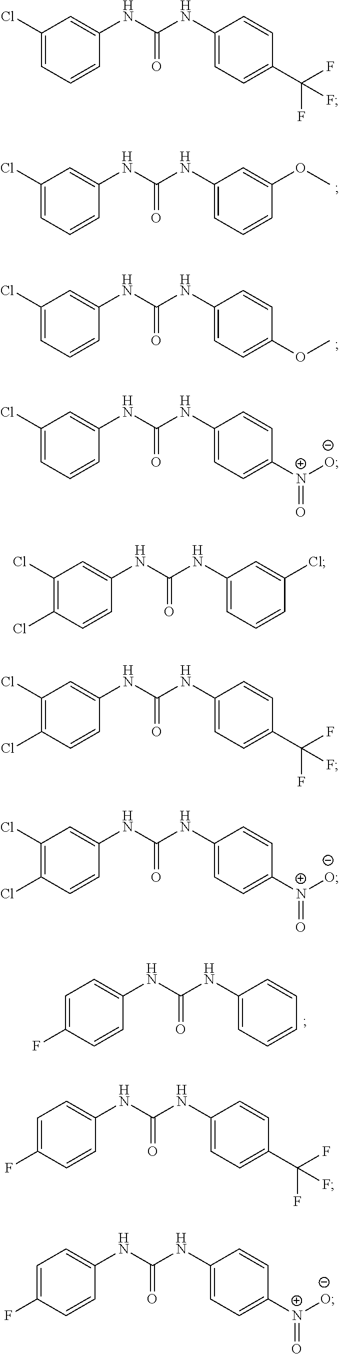 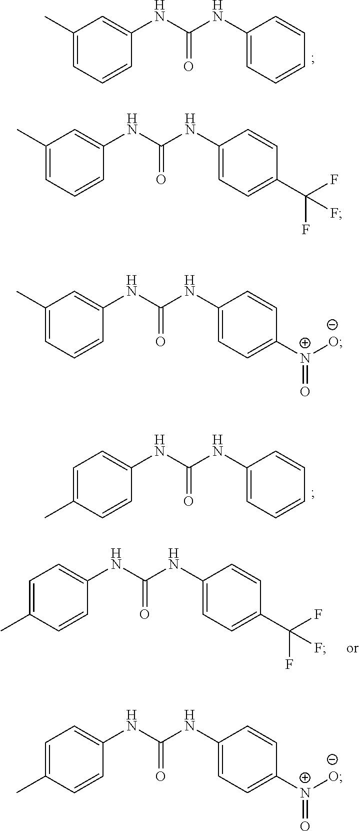
the compound is not 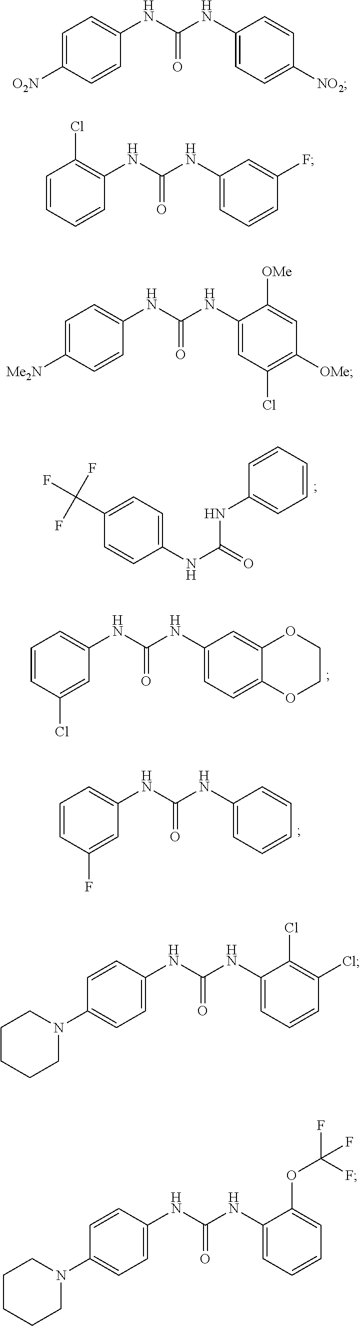 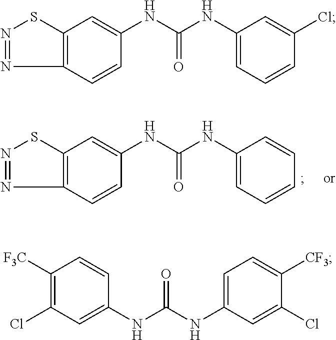 [SMILES: O=C(Nc1cccc(Cl)c1)Nc1ccc2nnsc2c1 ; O=C(Nc1ccccc1)Nc1ccc2nnsc2c1 ; O=C(Nc1ccc(C(F)(F)F)c(Cl)c1)Nc1ccc(C(F)(F)F)c(Cl)c1]
at least one of R¹ to R⁷ is not H;
the compound is not 1-(6-methoxybenzo[d]thiazol-2-yl)-3-phenylurea;
the compound is not 1-(4-chlorophenyl)-3-(6-methoxybenzo[d]thiazol-2-yl)urea;
the compound is not 1-(3-fluorophenyl)-3-(6-methoxybenzo[d]thiazol-2-yl)urea;
the compound is not 1-(3,4-dichlorophenyl)-3-(6-methoxybenzo[d]thiazol-2-yl)urea;
the compound is not 1-(2,3-dichlorophenyl)-3-(6-methoxybenzo[d]thiazol-2-yl)urea;
the compound is not 1-(3-chlorophenyl)-3-(2-hydroxyphenyl)urea;
the compound is not 1-(4-chlorophenyl)-3-(2-hydroxyphenyl)urea;
the compound is not 1-(3-fluorophenyl)-3-(6-methoxy-1,3-benzothiazol-2-yl)urea;
the compound is not 1,3-bis(3-chlorophenyl)urea;
the compound is not 1-(3-chlorophenyl)-3-(4-methylphenyl)urea; and
the compound is not 1-(3-chlorophenyl)-3-(2,4-dichlorophenyl)urea.

2. Use according to claim 1, wherein Z represents: 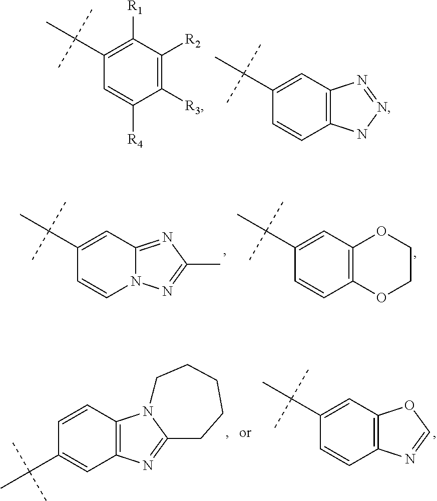
optionally wherein Z represents: 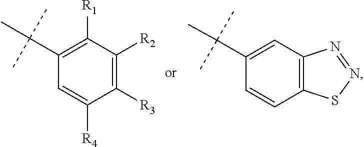
where the dotted lines represent the point of attachment to the rest of the molecule.

3. Use according to claim 1 or 2 wherein R¹ represents H, Cl, F, OCF₃, or OCH₃, optionally wherein R¹ represents H, Cl, F, or OCH₃.

4. Use according to any one of the preceding claims wherein R² represents H, Cl, or OCH₃.

5. Use according to any one of the preceding claims wherein R³ represents H, F, CF₃, CH₃, OCH₃, Cl, or NO₂, optionally wherein R³ represents H, F, CF₃, CH₃, OCH₃, or Cl.

6. Use according to any one of the preceding claims wherein R⁴ represents H or CH₃, optionally wherein R⁴ represents H.

7. Use according to any one of the preceding claims wherein R⁵ represents H or Cl, optionally wherein R⁵ represents H.

8. Use according to any one of the preceding claims wherein R⁶ represents H, CF₃, or NO₂, optionally wherein R⁶ represents H or NO₂.

9. Use according to any one of the preceding claims wherein R⁷ represents H.

10. Use according to any one of the preceding claims wherein R⁸ represents H.

11. Use according to any one of the preceding claims, wherein X represents NH.

12. Use according to any one of the preceding claims, wherein Y represents: 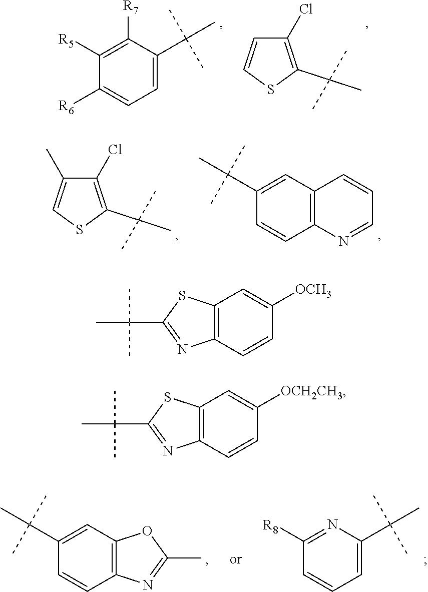
optionally wherein Y represents: 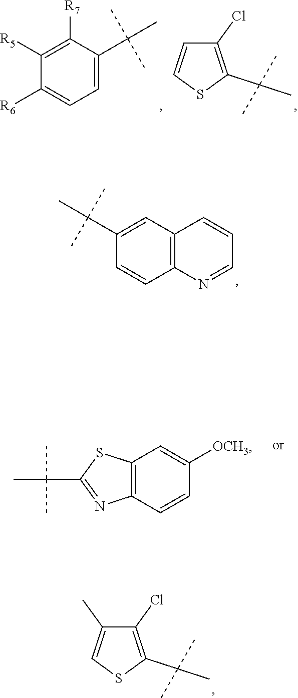
where the dotted lines represent the point of attachment to the rest of the molecule.

13. Use according to any one of claims 1 to 10, or use according to claim 12 as dependent on any one of claims 1 to 10, wherein X represents a bond.

14. Use according to any one of claims 1 to 10, or use according to claim 13 as dependent on any one of claims 1 to 10, wherein Y represents: 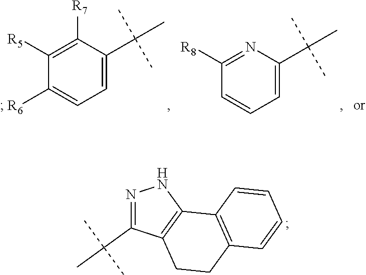
optionally wherein Y represents: 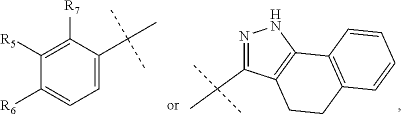
where the dotted lines represent the point of attachment to the rest of the molecule.

15. Use of a compound having the formula: 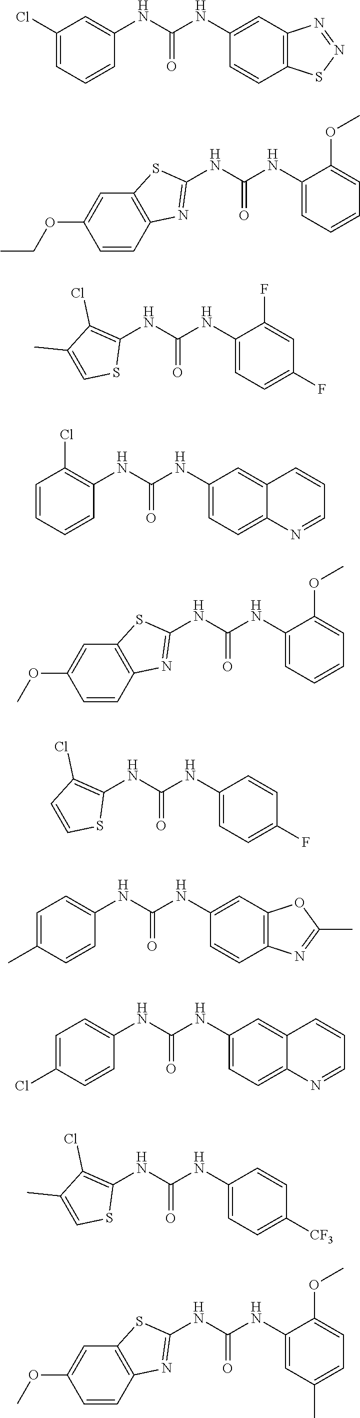 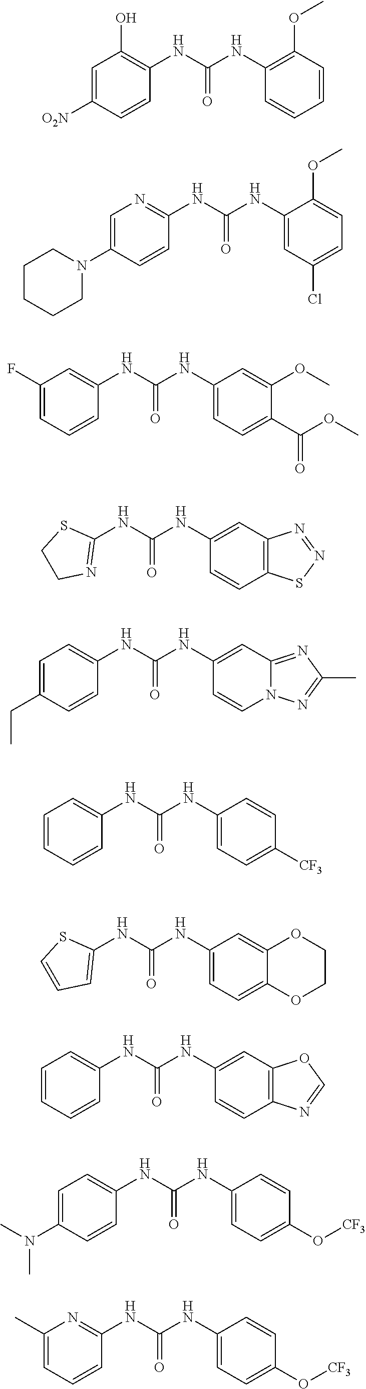 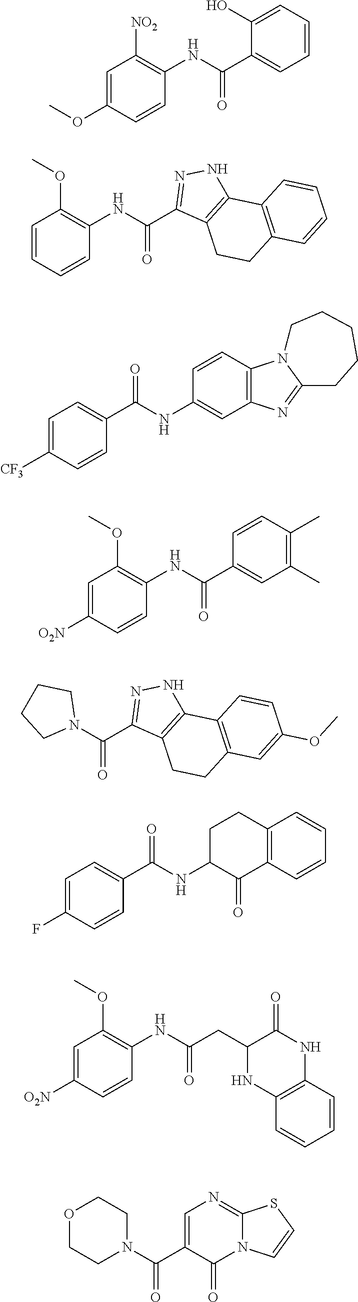 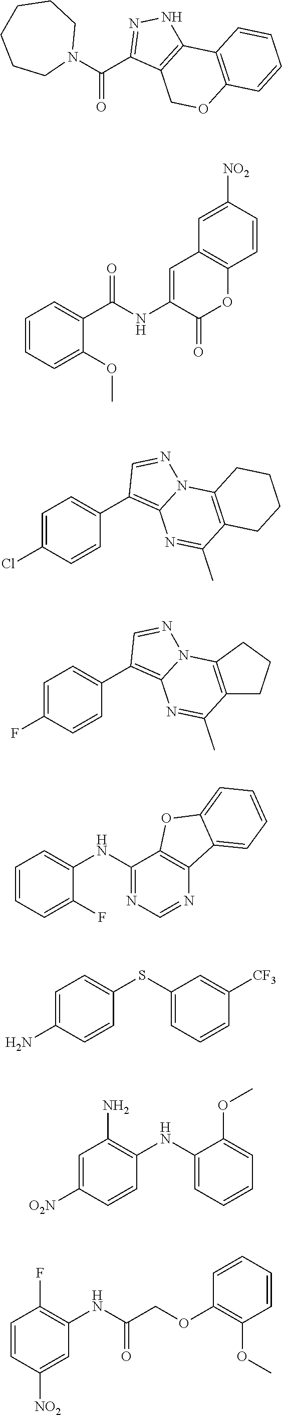
or a pharmaceutically acceptable salt, solvate or derivative thereof, in the preparation of a medicament to treat cancer.

16. Use according to any one of the preceding claims, wherein the cancer is selected from liver cancer (e.g. hepatocellular carcinoma) and lung cancer.
